(19) United States
(12) Patent Application Publication
(10) Pub. No.: US 2022/0002393 A1
(43) Pub. Date: Jan. 6, 2022

(54) METHODS FOR TREATMENT OF REFRACTORY GENERALIZED MYASTHENIA GRAVIS IN PEDIATRIC PATIENTS

(71) Applicant: Alexion Pharmaceuticals, Inc., Boston, MA (US)

(72) Inventors: Róisín ARMSTRONG, Mystic, CT (US); Kenji FUJITA, Milburn, NJ (US)

(21) Appl. No.: 17/293,688

(22) PCT Filed: Nov. 19, 2019
(86) PCT No.: PCT/US2019/062222

§ 371 Date: May 13, 2021

Related U.S. Application Data
(60) Provisional application No. 62/887,440, filed on Aug. 15, 2019.
(60) Provisional application No. 62/769,827, filed on Nov. 20, 2018.

(51) Int. Cl. C07K 16/18; A61P 21/04

(52) U.S. Cl. CPC C07K 16/18; A61K 2039/545; A61P 21/04

(57) ABSTRACT

The disclosure provides methods of treating myasthenia gravis (MG) in a pediatric subject in need thereof by administering to the subject a substance that specifically binds complement component 5 (CS). In son embodiments, the substance that specifically binds C5 is a binding protein, such as an anti-C5 antibody.

### RELATED APPLICATIONS

This application claims the benefit of priority to U.S. Provisional Patent Application No. 62/769,827, filed Nov. 20, 2018, and U.S. Provisional Patent Application No. 62/887,440, filed Aug. 15, 2019, the entire contents of which are incorporated herein by reference for all purposes.

### REFERENCE TO SEQUENCE LISTING SUBMITTED ELECTRONICALLY

The content of the electronically submitted sequence listing in ASCII text file (Name: 6017742_AX9_001PC_Sequence_Listing.txt; Size: 44 KB; and Date of Creation: Nov. 19, 2019) is incorporated herein by reference in its entirety.

### BACKGROUND

Myasthenia Gravis (MG) is a rare, debilitating, acquired autoimmune neurologic disorder of the neuromuscular junction (NMJ) caused by the failure of neuromuscular transmission, which results from the binding of auto-antibodies (Abs) to proteins involved in signaling at the NMJ. These proteins include the nicotine acetylcholine receptors (AChRs) or, less frequently, a muscle-specific tyrosine kinase (MuSK) involved in AChR clustering.

MG has a prevalence of 14-20 per 100,000 in the U.S., affecting roughly 60,000 Americans. It affects males and females in equal ratio, although the incidence in females peaks in the 3rd decade as compared to males in whom the peak age at onset is in the 6th or 7th decade. Mortality from MG is approximately 4%, mostly due to respiratory failure.

Myasthenia gravis is clinically characterized by weakness and fatigability of voluntary skeletal muscles. MG may initially present with ocular muscle weakness affecting eye and eyelid movement, referred to as ocular MG (oMG). Ten percent of subjects have disease limited to ocular muscles. Ninety percent of subjects have generalized MG, with muscle weakness involving neck, head, spine, bulbar, respiratory, or limb muscles. Bulbar weakness refers to muscles controlled by nerves originating from the bulb-like part of the brainstem and manifests as difficulty in talking, chewing, swallowing, and control of the head. MG may cause life-threatening respiratory failure, referred to as myasthenic crisis. About 15% to 20% of subjects will experience a myasthenic crisis during the course of their disease, 75% within 2 years of diagnosis, requiring hospitalization and ventilatory support.

While there is no cure for MG, there are a variety of therapies that reduce muscle weakness and improve neuromuscular function. Current available treatments for myasthenia gravis aim to modulate neuromuscular transmission, inhibit the production or effects of pathogenic antibodies, or inhibit inflammatory cytokines. There is currently no specific treatment that targets the underlying pathophysiology of NMJ injury specifically, i.e., anti-AChR antibody-AChR interactions resulting in complement activation via the classical pathway and inflammation, with the resultant destruction of the NMJ. There is no specific treatment that corrects the autoimmune defect in MG.

With immunosuppressive therapies (ISTs). the current standard of care, which usually combines cholinesterase inhibitors, corticosteroids and immunosuppressive drugs (most commonly azathioprine [AZA], cyclosporine, and mycophenolate mofetil [MMF]), the majority of subjects with MG have their disease reasonably well controlled. However, there is a cohort of refractory subjects who do not respond adequately to ISTs, or cannot tolerate ISTs, and those who require repeated treatments with plasma exchange (PE) and/or intravenous immunoglobulin (IVIg) to maintain clinical stability. For these subjects, an alternative therapy is needed.

### SUMMARY

This disclosure provides methods of treating refractory generalized myasthenia gravis in a pediatric patient in need thereof comprising administering a therapeutically effective amount of an anti-complement component 5 (C5) antibody or an antigen binding fragment thereof to the patient, wherein the patient is administered the anti-CS antibody or antigen binding fragment thereof for at least 26 weeks.

In some embodiments, this disclosure provides a method of treating refractory generalized myasthenia gravis in a pediatric patient in need thereof comprising administering a therapeutically effective amount of an anti-C5 antibody or an antigen binding fragment thereof to the patient, wherein the anti-C5 antibody, or an antigen binding fragment thereof is eculizumab or an eculizumab variant and wherein the patient is administered eculizumab or eculizumab variant for at least 26 weeks.

In some embodiments, this disclosure provides a method comprising administering a therapeutically effective amount of eculizumab to a pediatric patient, wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and wherein the patient is administered eculizumab for at least 26 weeks.

In some embodiments, this disclosure provides a method of treating refractory generalized myasthenia gravis in a patient in need thereof comprising administering eculizumab to the patient, wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) or requires chronic plasma exchange or chronic IVIg to maintain clinical stability; wherein eculizumab is administered using a phased dosing schedule with an induction phase comprising administering a 300 mg to 1200 mg induction dose of eculizumab.

In some embodiments, for a patient weighing ≥10 and <20 kg, the induction phase comprises administering a first dose of 600 mg eculizumab to the patient on Day 1.

In some embodiments, for a patient weighing ≥20 and <30 kg, the induction phase comprises administering a first dose of 600 mg eculizumab to the patient on Day 1, and a second dose of 600 mg eculizumab at least 7 days thereafter.

In some embodiments, for a patient weighing ≥30 and <40 kg, the induction phase comprises administering a first dose of 600 mg eculizumab to the patient on Day 1, and a second dose of 600 mg eculizumab at least 7 days thereafter.

In some embodiments, for a patient weighing ≥40 kg, the induction phase comprises administering a first dose of eculizumab 900 mg to the patient on Day 1, and a second dose, a third dose, and a fourth dose of 900 mg eculizumab 7, 14, and 21 days thereafter, respectively.

In some embodiments, this disclosure provides a method wherein the induction phase of eculizumab treatment is followed by a maintenance phase comprising administering 300 mg to 1200 mg of eculizumab.

In some embodiments, for a patient weighing ≥10 and <20 kg, the maintenance phase comprises administering a first dose of 300 mg eculizumab to the patient at Week 1, and subsequent doses of 300 mg eculizumab every 2 weeks thereafter.

In some embodiments, for a patient weighing ≥20 and <30 kg, the maintenance phase comprises administering a first dose of 600 mg eculizumab to the patient at Week 2, and subsequent doses of 600 mg eculizumab every 2 weeks thereafter.

In some embodiments, for a patient weighing ≥30 and <40 kg, the maintenance phase comprises administering a first dose of 900 mg eculizumab to the patient at Week 2, and subsequent doses of 900 mg eculizumab every 2 weeks thereafter.

In some embodiments, wherein for a patient weighing ≥40 kg, the maintenance phase comprises administering a first dose of 1200 mg eculizumab to the patient at Week 4, and subsequent doses of 1200 mg eculizumab every 2 weeks thereafter.

In some embodiments, the patient being treated by the methods provided herein experiences a clinically meaningful improvement (reduction) in Myasthenia Gravis Activities of Daily Living (MG-ADL) score after 26 weeks of treatment.

In some embodiments, the patient being treated by the methods provided herein experiences a clinically meaningful improvement (reduction) in quantitative Myasthenia Gravis score (QMG) after 26 weeks of treatment.

In some embodiments, the patient being treated by the methods provided herein experiences a clinically meaningful improvement (reduction) in Myasthenia Gravis Composite (MGC) score after 26 weeks of treatment.

In some embodiments, the patient being treated by the methods provided herein experiences a clinically meaningful improvement (reduction) in quality of life as measured by the Myasthenia Gravis Quality of Life (MG-QOL-15) score after 26 weeks of treatment.

In some embodiments, the patient being treated by the methods provided herein experiences a clinically meaningful improvement (reduction) in neuro-fatigue as measured by the Neuro-QOL Fatigue score after 26 weeks of treatment.

In some embodiments, the patient being treated by the methods provided herein experiences a clinically meaningful improvement (increase) in health status as measured by the EQ-5D health status score after 26 weeks of treatment.

In some embodiments, the patient has a QMG total score ≥12 prior to administering the therapeutically effective amount of eculizumab to the patient.

In some embodiments, this disclosure provides a method of treating refractory generalized myasthenia gravis in a patient in need thereof comprising administering eculizumab by intravenous infusion. In some embodiments, eculizumab is administered subcutaneously. In some embodiments, the eculizumab comprises a heavy chain amino acid sequence according to SEQ ID NO: 10 and a light chain amino acid sequence according to SEQ ID NO: 11. In some embodiments, the eculizumab is an eculizumab variant comprising a heavy chain amino acid sequence according to SEQ ID NO: 14 and a light chain amino acid sequence according to SEQ ID NO: 11. In some embodiments, the eculizumab is an eculizumab variant comprising a heavy chain variable region amino acid sequence according to SEQ ID NO: 12 and a light chain amino acid sequence according to SEQ ID NO: 11.

In some embodiments, this disclosure provides a method of treating refractory generalized myasthenia gravis in a patient in need thereof comprising administering an anti-C5 antibody, or antigen binding fragment thereof, wherein the antibody is an anti-C5 antibody or an antigen binding fragment thereof comprising a heavy chain variable region amino acid sequence according to SEQ ID NO: 27 and a light chain variable region amino acid sequence according to SEQ ID NO: 28. In some embodiments, the antibody is an anti-C5 antibody or an antigen binding fragment thereof comprising a heavy chain variable region amino acid sequence according to SEQ ID NO: 35 and a light chain variable region amino acid sequence according to SEQ ID NO: 36. In some embodiments, the antibody is an anti-C5 antibody or antigen binding fragment thereof comprising a heavy chain variable region amino acid sequence according to SEQ ID NO: 37 and a light chain variable region amino acid sequence according to SEQ ID NO: 38.

In some embodiments, this disclosure provides a method of treating refractory generalized myasthenia gravis in a pediatric patient in need thereof comprising administering an anti-C5 antibody or antigen binding fragment thereof, wherein the patient has failed treatment over one year or more with two or more ISTs in sequence or in combination.

In some embodiments, this disclosure provides a method of treating refractory generalized myasthenia gravis in a pediatric patient in need thereof comprising administering an anti-C5 antibody or antigen binding fragment thereof, wherein the patient has failed at least one IST and requires chronic plasma exchange or IVIg to control symptoms of myasthenia gravis.

In some embodiments, this disclosure provides a method of treating refractory generalized myasthenia gravis in a pediatric patient in need thereof comprising administering a therapeutically effective amount of eculizumab is maintained at a concentration of between 50-100 μg/mL in the patient's serum.

In some embodiments, this disclosure provides a method of treating refractory generalized myasthenia gravis in a pediatric patient in need thereof comprising administering a therapeutically effective amount of eculizumab, wherein the patient experiences a discontinuation in the administration of one or more IST following at least 26 weeks of treatment.

In some embodiments, this disclosure provides a method of treating refractory generalized myasthenia gravis in a pediatric patient in need thereof comprising administering a therapeutically effective amount of eculizumab, wherein the patient experiences a reduction in IST dosing following at least 26 weeks of treatment.

The disclosure also provides eculizumab for use in the treatment of refractory generalized myasthenia gravis in a pediatric patient according to any of the embodiments, described above.

Further, the disclosure encompasses any of the above embodiments being used with any other of the above embodiments in any combination.

### BRIEF DESCRIPTION OF THE DRAWINGS

FIG. 1 is a schematic of the overall design of the clinical trial disclosed herein.

FIG. 2 is a schematic representation of the pediatric Quantitative Myasthenia Gravis (QMG) score used in the clinical trial disclosed herein.

FIG. 3 is a schematic representation of the European Quality of Life 5-Dimension (EQ-5D-Y) questionnaire used in the clinical trial disclosed herein.

FIG. 4 is a schematic representation of the pediatric EQ-5D-Y questionnaire used in the clinical trial disclosed herein.

FIG. 5 is a schematic representation of the Neurological Quality of Life (Neuro-QoL) fatigue questionnaire used in the clinical trial disclosed herein.

FIG. 6 is a schematic representation of the pediatric neurological quality of life (Neuro-QoL) fatigue questionnaire used in the clinical trial disclosed herein.

FIG. 7 is a schematic representation of the Myasthenia Gravis Foundation of America (MGFA) clinical classification.

FIG. 8 is a schematic representation of the MGFA therapy status.

FIG. 9 is a schematic representation of the MGFA post-intervention status assessment used in the clinical trial disclosed herein.

FIG. 10 is a schematic representation of the laboratory panels and tests performed in the clinical trial disclosed herein.

FIG. 11 is a schematic representation of the post-treatment, end of study questionnaire used in the clinical trial disclosed herein.

### DETAILED DESCRIPTION

The disclosure provides methods of treating myasthenia gravis (MG) in pediatric subjects or patients in need thereof by administering an antibody that specifically binds complement component 5 (CS). In some embodiments, the antibody that specifically binds C5 reduces the rate at which C5 is cleaved, in vivo, into C5a and C5b. In some embodiments, the antibody that specifically binds C5, binds to one or both of the C5a and/or C5b fragments. In any of these embodiments, the antibody that specifically binds C5 blocks the complement cascade at C5, thereby reducing the release of proinflammatory mediators such as C5a and the formation of a C5b−9 Membrane Attack Complex (MAC).

In some embodiments, the antibody that specifically binds C5 is eculizumab. In some embodiments, eculizumab is an antibody or a fragment thereof.

Eculizumab (h5G1.1-mAb) is a humanized monoclonal antibody (mAb) that was derived from the murine anti-human C5 antibody m5G1.1. Eculizumab specifically binds the terminal complement protein C5, thereby inhibiting its cleavage to C5a and C5b during complement activation. This strategic blockade of the complement cascade at C5 prevents the release of proinflammatory mediators and the formation of the Membrane Attack Complex or cytolytic pore, while preserving the early components of complement activation that are essential for the opsonization of microorganisms and clearance of immune complexes.

C5 binding proteins are described in U.S. Pat. No. 6,355,245, which is hereby incorporated herein by reference in its entirety. In some embodiments, the anti-C5 antibody is a monoclonal antibody having a hybrid IgG2/4 isotype. In some embodiments, the anti-C5 antibodies are effective in reducing the cell-lysing ability of complement present in human blood. This property of the antibodies can be determined by methods well known in the art such as, for example, by the chicken erythrocyte hemolysis method described in U.S. Pat. No. 6,355,245.

In some embodiments, anti-C5 antibodies bind to C5 or fragments thereof, e.g., C5a or C5b. In some embodiments, the anti-C5 antibodies recognize and bind epitopes on either the alpha chain or the beta chain of purified human complement component C5 and are capable of blocking the conversion of C5 into C5a and C5b by C5 convertase. See Wurzner et al., Complement. Inflamm. 8(5-6): 328-40 (1991).

In some embodiments, the anti-C5 antibodies recognize and bind epitopes within the alpha chain of purified human complement component C5. In some embodiments, the antibodies are capable of blocking the conversion of C5 into C5a and C5b by C5 convertase. In some embodiments, the antibodies can provide this blockade at substantially the same concentrations needed to block hemolytic activity.

In some embodiments, the antibodies specifically bind to an amino-terminal region within the alpha chain, however, they do not specifically bind to free C5a. In some embodiments, the C5 antibody is able to substantially inhibit complement hemolytic activity and to substantially inhibit the conversion of C5 to produce C5a. In some embodiments, the C5 antibodies provide these functions when used at a molar ratio of antibody to antigen (C5) of 3:1 or less.

As used herein, the term “about” refers to an amount plus or minus 5% of a given value. For example, about 100 kg is 95-105 kg.

As used herein, the term “antibodies” refers to immunoglobulins produced in vivo, as well as those produced in vitro by a hybridoma, and antigen binding fragments (e.g., Fab′ preparations) of such immunoglobulins, as well as to recombinantly expressed antibodies or antigen binding proteins, including immunoglobulins, chimeric immunoglobulins, “humanized” immunoglobulins, antigen binding fragments of such immunoglobulins, single chain antibodies, and other recombinant proteins containing antigen binding domains derived from immunoglobulins such as DVD-Ig and CODV-Ig. See U.S. Pat. Nos. 7,161,181 and 9,181,349. “Specificity” refers to the ability of a binding protein to selectively recognize and bind an antigen at a particular location or structure, known as an epitope, often found on the surface of the antigen.

The term “specifically binds,” means that a binding protein or fragment thereof forms a complex with an antigen that is relatively stable under physiologic conditions. Specific binding can be characterized by a dissociation constant of at least about 1×10⁻⁶ M or smaller. In some embodiments, the dissociation constant is at least about 1×10⁻⁷ M, 1×10⁻⁸ M, 1×10⁻⁹ M, or 1×10⁻¹⁰ M. Methods for determining whether two molecules specifically bind are well known in the art and include, for example, equilibrium dialysis, surface plasmon resonance, and the like.

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the invention can be generated using methods known in the art.

An exemplary anti-C5 antibody is eculizumab comprising heavy and light chains having the sequences shown in SEQ ID NOs: 10 and 11, respectively, or antigen binding fragments and variants thereof. Eculizumab (also known as SOLIRIS*) is described in U.S. Pat. No. 6,355,245. Eculizumab is a humanized monoclonal antibody that is a terminal complement inhibitor.

In some embodiments, the antibody comprises the heavy and light chain complementarity determining regions (CDRs) or variable regions of eculizumab. Accordingly, in some embodiments, the antibody comprises the CDR1, CDR2, and CDR3 domains of the VH region of eculizumab having the sequence set forth in SEQ ID NO: 7, and the CDR1, CDR2, and CDR3 domains of the VL region of eculizumab having the sequence set forth in SEQ ID NO: 8. In some embodiments, the antibody comprises heavy chain CDR1, CDR2, and CDR3 domains having the sequences set forth in SEQ ID NOs: 1, 2, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 7 and SEQ ID NO: 8, respectively.

As used herein, a “therapeutically effective amount” is a dosage of therapeutic that when administered alleviates at least one symptom of a pathology. In some embodiments, a therapeutically effective amount of eculizumab is a dosage that alleviates at least one symptom of refractory generalized myasthenia gravis in a pediatric subject.

Empirical data indicate that serum eculizumab concentrations greater than 50 μg/mL and closer to at least 100 μg/mL are required to significantly reduce free C5 concentrations. Specifically, free C5 concentration was reduced significantly with increasing concentrations of eculizumab beginning at >50 μg/mL and was at near zero levels with eculizumab concentrations above 100 μg/ml. Thus, in some embodiments, the method comprises administering a therapeutically effective amount of eculizumab to the subject, wherein the therapeutically effective amount of eculizumab is maintained at a concentration of at least 50 μg/mL of eculizumab in serum of the subject. In some embodiments, the method comprises administering a therapeutically effective amount of eculizumab to the subject, wherein the therapeutically effective amount of eculizumab is maintained at a concentration of at least 60 μg/mL of eculizumab in serum of the subject. In some embodiments, the method comprises administering a therapeutically effective amount of eculizumab to the subject, wherein the therapeutically effective amount of eculizumab is maintained at a concentration of at least 70 μg/mL of eculizumab in serum of the subject. In some embodiments, the method comprises administering a therapeutically effective amount of eculizumab to the subject, wherein the therapeutically effective amount of eculizumab is maintained at a concentration of at least 80 μg/mL of eculizumab in serum of the subject. In some embodiments, the method comprises administering a therapeutically effective amount of eculizumab to the subject, wherein the therapeutically effective amount of eculizumab is maintained at a concentration of at least 90 μg/mL of eculizumab in serum of the subject. In some embodiments, the method comprises administering a therapeutically effective amount of eculizumab to the subject, wherein the therapeutically effective amount of eculizumab is maintained at a concentration of at least 100 μg/mL of eculizumab in serum of the subject.

Another exemplary anti-C5 antibody is an eculizumab variant, known as antibody BNJ441, and engineered to have a longer half-life (T½) in humans comprising heavy and light chains having the sequences shown in SEQ ID NOs: 14 and 11, respectively, or antigen binding fragments and variants thereof. BNJ441 (also known as ALXN1210) is described in International Publication No. WO 2015/134894 A1 and U.S. Pat. No. 9,079,949, the teachings or which are hereby incorporated by reference. BNJ441 is a humanized monoclonal antibody that is structurally related to eculizumab (SOLRIS®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In some embodiments, the antibody comprises the heavy and light chain CDRs or variable regions of BNJ441. Accordingly, in some embodiments, the antibody comprises the CDR1, CDR2, and CDR3 domains of the VH region of BNJ441 having the sequence set forth in SEQ ID NO: 12, and the CDR1, CDR2, and CDR3 domains of the VL region of BNJ441 having the sequence set forth in SEQ ID NO: 8. In some embodiments, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 12 and SEQ ID NO: 8, respectively. In some embodiments, the antibody may comprise the heavy chain constant region of BNJ441 having the amino acid sequence set forth in SEQ ID NO: 13.

In some embodiments, eculizumab is administered in a multiphase dosing regimen. For example, the multiphase dosing regimen comprises a first phase and a second phase in some embodiments. In some embodiments, the first phase is an induction phase and comprises administration of eculizumab at between 300 mg and 1200 mg once a week to the subject for between 1-10 weeks. The induction phase is concluded by administering the first maintenance phase dose of between 300 mg and 1200 mg one week after the last induction dose.

In some embodiments, the second phase is a maintenance phase and comprises administration of eculizumab at between 300 mg and 1200 mg once every two weeks to the subject for 2 weeks, 4 weeks, 6 weeks, 8 weeks, 12, weeks, 26 weeks, or as long as myasthenia gravis persists. In some embodiments, the maintenance phase comprises administration of eculizumab at between 300 mg and 1200 mg once every two weeks to the subject for 2 months, 4 months, 6 months, 8 months, 12 months, 2 years, three years, 4 years, 5 years, or for the remaining lifetime of the patient. In some embodiments, the maintenance phase comprises administration of eculizumab at about between 300 mg and 1200 mg twice a month (biweekly) once the induction phase is complete.

In some embodiments, the method comprises administering to a patient an effective amount of eculizumab, or antigen binding fragment thereof, wherein the effective amount is based on the weight of the patient. For example, in some embodiments, about 150 mg, about 300 mg, about 450 mg, about 600 mg, about 750 mg, about 900 mg, about 1050 mg, about 1200 mg, about 1350 mg, about 1500 mg, about 1650 mg, about 1800 mg, or about 1950 mg of eculizumab, or an antigen binding fragment thereof, is administered to a patient based on their weight. In some embodiments, dosage regimens are adjusted to provide the optimum desired response (e.g., an effective dose response).

In some embodiments, these doses are provided to patients in a phased dosing regimen. In some embodiments, the phased dosing regimen includes an induction and a maintenance phase. In some embodiments, the number of doses provided in a given period are higher during the induction phase than in the maintenance phase. In some embodiments, the number of doses provided in the induction phase are twice as many in a given period then in the maintenance phase. In some embodiments, during the induction phase doses are provided every week and in the maintenance phase, doses are provided every other week. Any of the above doses can be provided in either the induction or maintenance phase.

In some embodiments, 150 mg to 750 mg of eculizumab, or an antigen binding fragment thereof, is administered during an administration cycle to a patient weighing ≥10 and <20 kg. In some embodiments, 150 mg to 900 mg of eculizumab, or an antigen binding fragment thereof, is administered during an administration cycle to a patient weighing ≥20 and <30 kg. In some embodiments, 150 mg to 1050 mg of eculizumab, or an antigen binding fragment thereof, is administered during an administration cycle to a patient weighing ≥30 and <40 kg. In some embodiments, 450 mg to 1350 mg of eculizumab, or an antigen binding fragment thereof, is administered during an administration cycle to a patient weighing ≥40 kg. In some embodiments, the administration cycle is either the induction phase or the maintenance phase.

In some embodiments, the method comprises administering to a patient during an induction phase of an administration cycle an effective amount of eculizumab or antigen binding fragment thereof, wherein: for a patient weighing ≥10 and <20 kg, a first dose of about 600 mg eculizumab is administered to the patient on Day 1; for a patient weighing 20 and <30 kg, a first dose of about 600 mg eculizumab is administered to the patient on Day 1, and a second dose of about 600 mg eculizumab is administered to the patent at least 7 days thereafter; for a patient weighing ≥30 and <40 kg, a first dose of about 600 mg eculizumab is administered to the patient on Day 1, and a second dose of about 600 mg eculizumab is administered to the patent at least 7 days thereafter; and for a patient weighing ≥40 kg, a first dose of eculizumab about 900 mg is administered to the patient on Day 1, and a second, third, and fourth dose of about 900 mg eculizumab is administered to the patent 7, 14, and 21 days thereafter, respectively.

In some embodiments, the method comprises administering to a patient during an induction phase of an administration cycle an effective amount of eculizumab or antigen binding fragment thereof, wherein: for a patient weighing ≥10 and <20 kg, a first dose of 600 mg eculizumab is administered to the patient on Day 1; for a patient weighing ≥20 and <30 kg, a first dose of 600 mg eculizumab is administered to the patient on Day 1, and a second dose of 600 mg eculizumab is administered to the patent at least 7 days thereafter; for a patient weighing ≥30 and <40 kg, a first dose of 600 mg eculizumab is administered to the patient on Day 1, and a second dose of 600 mg eculizumab is administered to the patent at least 7 days thereafter; and for a patient weighing ≥40 kg, a first dose of eculizumab 900 mg is administered to the patient on Day 1, and a second, third, and fourth dose of 900 mg eculizumab is administered to the patent 7, 14, and 21 days thereafter, respectively.

In some embodiments, the method comprises administering to a patient during a maintenance phase of an administration cycle an effective amount of eculizumab or antigen binding fragment thereof, wherein: for a patient weighing ≥10 and <20 kg, a first dose of about 300 mg eculizumab is administered to the patient at Week 1, and subsequent doses of about 300 mg eculizumab are administered to the patient every 2 weeks thereafter; for a patient weighing ≥20 and <30 kg, a first dose of about 600 mg eculizumab is administered to the patient at Week 2, and subsequent doses of about 600 mg eculizumab are administered to the patient every 2 weeks thereafter; for a patient weighing ≥30 and <40 kg, a first dose of about 900 mg eculizumab is administered to the patient at Week 2, and subsequent doses of about 900 mg eculizumab are administered to the patient every 2 weeks thereafter; and for a patient weighing ≥40 kg, a first dose of about 1200 mg eculizumab is administered to the patient at Week 4, and subsequent doses of about 1200 mg eculizumab are administered to the patient every 2 weeks thereafter.

In some embodiments, the method comprises administering to a patient during a maintenance phase of an administration cycle an effective amount of eculizumab or antigen binding fragment thereof, wherein: for a patient weighing ≥10 and <20 kg, a first dose of 300 mg eculizumab is administered to the patient at Week 1, and subsequent doses of 300 mg eculizumab are administered to the patient every 2 weeks thereafter; for a patient weighing ≥20 and <30 kg, a first dose of 600 mg eculizumab is administered to the patient at Week 2, and subsequent doses of 600 mg eculizumab are administered to the patient every 2 weeks thereafter; for a patient weighing ≥30 and <40 kg, a first dose of 900 mg eculizumab is administered to the patient at Week 2, and subsequent doses of 900 mg eculizumab are administered to the patient every 2 weeks thereafter; and for a patient weighing ≥40 kg, a first dose of 1200 mg eculizumab is administered to the patient at Week 4, and subsequent doses of 1200 mg eculizumab are administered to the patient every 2 weeks thereafter.

In some embodiments, the method comprises administering to a patient receiving maintenance IVIg during an administration cycle an effective supplemental amount of eculizumab, or antigen binding fragment thereof, wherein: for a patient weighing ≥10 and <20 kg, the effective supplemental amount comprises about 300 mg eculizumab during the induction phase or maintenance phase; for a patient weighing ≥20 and <30 kg, the effective supplemental amount comprises about 300 mg eculizumab during the induction phase or maintenance phase; for a patient weighing ≥30 and <40 kg, the effective supplemental amount comprises about 300 mg during the induction phase or about 600 mg during the maintenance phase; and for a patient weighing ≥40 kg, the effective supplement amount comprises about 600 mg during the induction or maintenance phases.

In some embodiments, the method comprises administering to a patient receiving maintenance IVIg during an administration cycle an effective supplemental amount of eculizumab, or antigen binding fragment thereof, wherein: for a patient weighing ≥10 and <20 kg, the effective supplemental amount comprises 300 mg eculizumab during the induction phase or maintenance phase; for a patient weighing ≥20 and <30 kg, the effective supplemental amount comprises 300 mg eculizumab during the induction phase or maintenance phase; for a patient weighing ≥30 and <40 kg, the effective supplemental amount comprises 300 mg during the induction phase or 600 mg during the maintenance phase; and for a patient weighing ≥40 kg, the effective supplement amount comprises 600 mg during the induction or maintenance phases.

In some embodiments, the method comprises administering a therapeutically effective amount of eculizumab or an eculizumab variant to the subject, wherein the therapeutically effective amount of eculizumab or eculizumab variant is maintained at a concentration of between 50-100 μg/mL, between 60-100 μg/mL, between 70-100 μg/mL, between 80-100 μg/mL, or between 90-100 μg/mL of eculizumab in serum of the subject.

Another exemplary anti-C5 antibody is antibody BNJ421 comprising heavy and light chains having the sequences shown in SEQ ID NOs: 20 and 11, respectively, or antigen binding fragments and variants thereof. BNJ421 (also known as ALXN1211) is described in International Publication No. WO 2015/134894 A1 and U.S. Pat. No. 9,079,949, the teachings or which are hereby incorporated by reference.

In some embodiments, the antibody comprises the heavy and light chain CDRs or variable regions of BNJ421. Accordingly, in some embodiments, the antibody comprises the CDR1, CDR2, and CDR3 domains of the VH region of BNJ421 having the sequence set forth in SEQ ID NO: 12, and the CDR1, CDR2, and CDR3 domains of the VL region of BNJ421 having the sequence set forth in SEQ ID NO: 8. In some embodiments, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 12 and SEQ ID NO: 8, respectively. In some embodiments, the antibody may comprise the heavy chain constant region of BNJ421 having the amino acid sequence set forth in SEQ ID NO: 9.

Another exemplary anti-C5 antibody is the 7086 antibody described in U.S. Pat. Nos. 8,241,628 and 8,883,158. In some embodiments, the antibody may comprise the heavy and light chain CDRs or variable regions of the 7086 antibody. See U.S. Pat. Nos. 8,241,628 and 8,883,158. In some embodiments, the antibody, or a fragment thereof, may comprise heavy chain CDR1, CDR2, and CDR3 domains having the sequences set forth in SEQ ID NOs: 21, 22, and 23, respectively, and light chain CDR1, CDR2, and CDR3 domains having the sequences set forth in SEQ ID NOs: 24, 25, and 26, respectively. In some embodiments, the antibody or fragment thereof may comprise the VH region of the 7086 antibody having the sequence set forth in SEQ ID NO: 27, and the VL region of the 7086 antibody having the sequence set forth in SEQ ID NO: 28.

Another exemplary anti-C5 antibody is the 8110 antibody also described in U.S. Pat. Nos. 8,241,628 and 8,883,158. In some embodiments, the antibody may comprise the heavy and light chain CDRs or variable regions of the 8110 antibody. The antibody, or fragment thereof may comprise heavy chain CDR1, CDR2, and CDR3 domains having the sequences set forth in SEQ ID NOs: 29, 30, and 31, respectively, and light chain CDR1, CDR2, and CDR3 domains having the sequences set forth in SEQ ID NOs: 32, 33, and 34, respectively. In some embodiments, the antibody may comprise the VH region of the 8110 antibody having the sequence set forth in SEQ ID NO: 35, and the VL region of the 8110 antibody having the sequence set forth in SEQ ID NO: 36.

Another exemplary anti-C5 antibody comprises a heavy chain variable region amino acid sequence according to SEQ ID NO: 37 and a light chain variable region amino acid sequence according to SEQ ID NO: 38.

In some embodiments, eculizumab, an eculizumab variant such as BNJ441, or other anti-C5 antibody is administered to the subject once a month, once every two months, or once every three months depending on the dose. In some embodiments, the eculizumab, eculizumab variant such as BNJ441, or other anti-C5 antibody is administered once every two weeks, once a week, twice a week, or three times a week. In some embodiments, eculizumab, eculizumab variant such as BNJ441, or other anti-C5 antibody is administered once a week, once every two weeks, once every three weeks, once every four weeks, once every five weeks, once every six weeks, or once every eight weeks depending on the needs of the patient. In some embodiments, eculizumab, eculizumab variant such as BNJ441, or other anti-C5 antibody in administered intravenously (IV) or subcutaneously (SubQ).

Also, provided herein are pharmaceutical compositions comprising an anti-C5 antibody or antigen binding fragment thereof with a pharmaceutically acceptable excipient for treating MG. In some embodiments, the composition comprises an antibody comprising the CDR1, CDR2, and CDR3 domains of the VH region of eculizumab having the sequence set forth in SEQ ID NO: 7, and the CDR1, CDR2, and CDR3 domains of the VL region of eculizumab having the sequence set forth in SEQ ID NO: 8. In some embodiments, the antibody comprises heavy chain CDR1, CDR2, and CDR3 domains having the sequences set forth in SEQ ID NOs: 1, 2, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 7 and SEQ ID NO: 8, respectively.

In some embodiments, the antibody comprises the heavy and light chain CDRs or variable regions of BNJ441. In some embodiments, the antibody comprises the CDR1, CDR2, and CDR3 domains of the VH region of BNJ441 having the sequence set forth in SEQ ID NO: 12, and the CDR1, CDR2, and CDR3 domains of the VL region of BNJ441 having the sequence set forth in SEQ ID NO: 8. In some embodiments, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 12 and SEQ ID NO: 8, respectively.

In some embodiments, the antibody comprises the heavy and light chain CDRs or variable regions of BNJ421. In some embodiments, the antibody comprises the CDR1, CDR2, and CDR3 domains of the VH region of BNJ421 having the sequence set forth in SEQ ID NO: 12, and the CDR1, CDR2, and CDR3 domains of the VL region of BNJ421 having the sequence set forth in SEQ ID NO: 8. In some embodiments, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In some embodiments, the antibody comprises VH and VL regions having the amino acid sequences set forth in SEQ ID NO: 12 and SEQ ID NO: 8, respectively.

### 1. Methods of Treating Myasthenia Gravis

The disclosure provides methods of treating pediatric subjects suffering from myasthenia gravis (MG) by administering an antibody that specifically binds C5. In some embodiments, the subject is a mammalian subject.

As used herein, the term “subject” and “patient” are interchangeable. In some embodiments, subjects and/or patients are mammals. According to some embodiments, primates include humans. Thus, in some embodiments, the subjects or patients suffering from MG described herein are humans.

As used herein, the term “pediatric subject” and “pediatric patient” are interchangeable. A pediatric patient or subject is a human subject <18 years of age. In some embodiments, a pediatric patient or subject is >6 years of age.

In some embodiments, MG includes refractory generalized myasthenia gravis. In some embodiments, refractory generalized myasthenia gravis is characterized as including subjects or patients positive for auto-antibodies binding to nicotinic acetylcholine receptor (anti-AChR) who continue to show marked generalized weakness or bulbar signs and symptoms of myasthenia gravis while receiving current standard of care for myasthenia gravis such as cholinesterase inhibitor therapy and immunosuppressant therapy (IST) or who require chronic plasma exchange or chronic IVIg to maintain clinical stability. In some embodiments, refractory generalized myasthenia gravis is characterized as including subjects or patients who continue to show marked generalized weakness or bulbar signs and symptoms of myasthenia gravis while receiving current standard of care for myasthenia gravis such as cholinesterase inhibitor therapy and immunosuppressant therapy (IST) or who require chronic plasma exchange or chronic IVIg to maintain clinical stability.

In some embodiments, MG includes refractory generalized myasthenia gravis. In some embodiments, refractory generalized myasthenia gravis is characterized as including subjects or patients positive for auto-antibodies binding to nicotinic acetylcholine receptor (anti-AChR) who continue to show marked generalized weakness or bulbar signs and symptoms of myasthenia gravis while receiving cholinesterase inhibitor therapy and immunosuppressant therapy (IST) and who require chronic plasma exchange or chronic IVIg to maintain clinical stability. In some embodiments, refractory generalized myasthenia gravis is characterized as including subjects or patients who continue to show marked generalized weakness or bulbar signs and symptoms of myasthenia gravis while receiving cholinesterase inhibitor therapy and immunosuppressant therapy (IST) and who require chronic plasma exchange or chronic IVIg to maintain clinical stability.

As used herein, the phrase “requires chronic plasma exchange” to maintain clinical stability refers to the use of plasma exchange therapy on a patient on a regular basis for the management of muscle weakness at least every 3 months over the last 12 months.

As used herein, the phrase “requires chronic IVIg” to maintain clinical stability refers to the use of IVIg therapy on a patient on a regular basis for the management of muscle weakness at least every 3 months over the last 12 months.

In some embodiments, treatment of MG includes the amelioration or improvement of one or more symptoms associated with MG. Symptoms associated with MG include muscle weakness and fatigability. Muscles primarily affected by MG include muscles that control eye and eyelid movement, facial expressions, chewing, talking, swallowing, breathing, neck movements, and limb movements.

In some embodiments, treatment of MG includes the improvement of a clinical marker for MG progression. These markers include MG activity of daily living profile (MG-ADL), quantitative Myasthenia Gravis (QMG) score for disease severity, Myasthenia Gravis composite (MGC), negative inspiratory force (NIF), forced vital capacity, MGFA post-intervention status, and other quality of life measurements. In some embodiments, MG-ADL is the primary score for measuring improvement of MG.

The MG-ADL is an 8-point questionnaire that focuses on relevant symptoms and functional performance of activities of daily living (ADL) in MG subjects (see Table 1). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MG-ADL would be a 3 point or greater reduction in score after 26 weeks of treatment.

The current QMG scoring system consists of 13 items: ocular (2 items), facial (1 item), bulbar (2 items), gross motor (6 items), axial (1 item), and respiratory (1 item); each graded 0 to 3, with 3 being the most severe (see Table 2). The range of total QMG score is 0-39. The QMG scoring system is considered to be an objective evaluation of therapy for MG and is based on quantitative testing of sentinel muscle groups. The MGFA task force has recommended that the QMG score be used in prospective studies of therapy for MG. A clinically meaningful improvement in a patient's QMG would be a 5 point or greater reduction in score after 26 weeks of treatment.

TABLE 1
MG ACTIVITY OF DAILY LIVING (MG-ADL) PROFILE

| | | | | | |
|---|---|---|---|---|---|
| Items | Grade 0 | Grade 1 | Grade 2 | Grade 3 | Score (0, 1, 2, 3) |

| | | | | |
|---|---|---|---|---|
| 1. Talking | Normal | Intermittent | Constant | Difficult to |
| | | slurring or | slurring or | understand |
| | | nasal speech | nasal, but can | speech |
| | | | be understood | |
| 2. Chewing | Normal | Fatigue with | Fatigue with | Gastric Tube |
| | | solid food | soft food | |
| 3. Swallowing | Normal | Rare episode of | Frequent | Gastric Tube |
| | | choking | choking | |
| | | | necessitating | |
| | | | changes in diet | |
| 4. Breathing | Normal | Shortness of | Shortness of | Ventilator |
| | | breath with | breath at rest | dependence |
| | | exertion | | |
| 5. Impairment of | None | Extra effort, | Rest periods | Cannot do |
| ability to brush | | but no rest | needed | one of these |
| teeth or comb hair | | periods needed | | functions |
| 6. Impairment of | None | Mild, | Moderate, | Severe, |
| ability to arise | | sometimes uses | always uses | requires |
| from a chair | | arms | arms | assistance |
| 7. Double vision | None | Occurs, but not | Daily, but not | Constant |
| | | daily | constant | |
| 8. Eyelid drop | None | Occurs, but not | Daily, but not | Constant |
| | | daily | constant | |

The MGC is a validated assessment tool for measuring clinical status of subjects with MG (16). The MGC assesses 10 important functional areas most frequently affected by MG and the scales are weighted for clinical significance that incorporates subject-reported outcomes. See Table 3. A clinically meaningful improvement in a patient's MGC would be a 3 point or greater reduction in score after 26 weeks of treatment.

TABLE 3
MG COMPOSITE SCALE

| | | | | | | | | |
|---|---|---|---|---|---|---|---|---|
| Ptosis, upward gaze (PE) | >45 seconds | 0 | 11-45 seconds | 1 | 1-10 seconds | 2 | Immediate | 3 |
| Double vision on lateral | >45 seconds | 0 | 11-45 seconds | 1 | 1-10 seconds | 2 | Immediate | 4 |
| gaze, left or right (PE) | | | | | | | | |
| Eye closure (PE) | Normal | 0 | Mild weakness | 0 | Moderate weakness | 1 | Severe weakness | 2 |
| | | | (can be forced | | (can be forced | | (unable to | |
| | | | open with effort) | | open easily) | | keep eyes closed) | |
| Talking (Pt) | Normal | 0 | Intermittent | 2 | Constant | 4 | Difficult to | 6 |
| | | | slurring or | | slurring or | | understand | |
| | | | nasal speech | | nasal but can | | | |
| | | | | | be understood | | | |
| Chewing (Pt) | Normal | 0 | Fatigue with | 2 | Fatigue with | 4 | Gastric tube | 6 |
| | | | solid food | | soft food | | | |
| Swallowing (Pt) | Normal | 0 | Rare trouble | 2 | Frequent trouble | 5 | Gastric tube | 6 |
| | | | or choking | | (change in diet) | | | |
| Breathing | Normal | 0 | SOB with | 2 | SOB at rest | 4 | Ventilator | 9 |
| | | | exertion | | | | | |
| Neck Flex/Ext (weakest PE) | Normal | 0 | Mild | 1 | Moderate (~50% | 3 | Severe | 4 |
| | | | | | weak +/− 15%) | | | |
| Shoulder Abd (PE | Normal | 0 | Mild | 2 | Moderate (~50% | 4 | Severe | 5 |
| | | | | | weak +/− 15%) | | | |
| Hip flexion | Normal | 0 | Mild | 2 | Moderate (~50% | 4 | Severe | 5 |
| | | | | | weak +/− 15%) | | | |
| | | 0 | | 15 | | 33 | | 50 |

The 15-item Myasthenia Gravis Qualify of Life 15 scale (MG-QOL 15) is a health-related quality of life evaluative instrument specific to subjects with MG. See Table 4. MG-QOL15 was designed to provide information about subjects' perception of impairment and disability and the degree to which disease manifestations are tolerated and to be easy to administer and interpret. The range of total scores is from 0 to 60. Higher scores translate into a greater extent of a patient's dissatisfaction with MG related dysfunction. The MG-QOL 15 is completed by the subject. Higher scores indicate greater extent of and dissatisfaction with MG-related dysfunction. A clinically meaningful improvement in a patient's MG-QOL 15 would be a decrease in score after 26 weeks of treatment.

TABLE 4
MYASTHENIA GRAVIS QUALIFY OF LIFE 15 SCALE (MG-QOL 15)

| Statement: How true in past 4 weeks? | Not at all | A little bit | Somewhat | Quite a bit | Very Much |
|---|---|---|---|---|---|
| Frustrated by condition | 0 | 1 | 2 | 3 | 4 |
| Trouble using my eyes | 0 | 1 | 2 | 3 | 4 |
| Trouble eating | 0 | 1 | 2 | 3 | 4 |
| Condition limits social life | 0 | 1 | 2 | 3 | 4 |
| Condition limits hobbies/fun | 0 | 1 | 2 | 3 | 4 |
| Trouble meeting family's needs | 0 | 1 | 2 | 3 | 4 |
| Need to plan around condition | 0 | 1 | 2 | 3 | 4 |
| Occupational skills/job negatively affected | 0 | 1 | 2 | 3 | 4 |
| Difficulty speaking | 0 | 1 | 2 | 3 | 4 |
| Trouble driving | 0 | 1 | 2 | 3 | 4 |
| Depressed about condition | 0 | 1 | 2 | 3 | 4 |
| Trouble walking | 0 | 1 | 2 | 3 | 4 |
| Trouble getting around in public places | 0 | 1 | 2 | 3 | 4 |
| Feel overwhelmed by condition | 0 | 1 | 2 | 3 | 4 |
| Trouble performing personal grooming | 0 | 1 | 2 | 3 | 4 |

The Neuro-QOL Fatigue is a reliable and validated brief 19-item survey of fatigue completed by the subject. Higher scores indicate greater fatigue and greater impact of MG on activities (see Table 5). A clinically meaningful improvement in a patient's Neuro-QQL Fatigue score would be reflected in a decrease in score after 26 weeks of treatment.

The EUROQOL (EQ-5D) is a reliable and validated survey of health status in 5 areas: mobility, self-care, usual activities, pain/discomfort, and anxiety/depression, completed by the subject. Each area has 3 levels: level 1 (no problems), level 2 (some problems), and level 3 (extreme problems). The EQ VAS records the subject's self-rated health on a vertical, 20 cm visual analogue scale where the endpoints are labeled “Best imaginable health state, marked as 100” and “Worst imaginable health state, marked as 0.” The EQ-5D is administered at Day 1, Weeks 4, 8, 12, 16, 20, and 26 or ET (Visits 2, 6, 8, 10, 12, 14, and 17 or ET). A clinically meaningful improvement in a patient's EQ-5D would be reflected as an increase in score after 26 weeks of treatment.

Subjects with increasingly severe MG can suffer from potentially fatal respiratory complications including profound respiratory muscle weakness. Respiratory function is monitored closely for evidence of respiratory failure in MG subjects and ventilator support is recommended in the event of consistent declines in serial measurements of Forced Vital Capacity (FVC) or Negative Inspiratory Force (NIF), loss of upper airway integrity (difficulty handling oral secretions, swallowing, or speaking) or in the setting of emerging respiratory failure. FVC as one of the test items in QMG is performed when QMG is performed. NIF was performed using the NIF Meter.

The MG clinical state is assessed using the MGFA Post-Intervention Status. Change in status categories of Improved, Unchanged, Worse, Exacerbation and Died of MG as well as the Minimal Manifestation (MM) can be assessed.

According to some embodiments, patients administered eculizumab show a reduced MG-ADL. In some embodiments, the subjects have an initial MG-ADL score of greater than 6 points. In some embodiments, the subjects have an initial MG-ADL score greater than 0, 1, 2, 3, 4, 5, 6, 7, 8, 9, 10, 11, 12, 13, 14, 15, 16, 17, 18, 19, 20, 21, 22, or 23 points. In some embodiments, after a course of treatment with eculizumab, the MG-ADL score of the subject has been reduced to less than 6 points. In some embodiments, the MG-ADL score has been reduced at least 1 point, at least 2 points, at least 3 points, at least 4 points, at least 5 points, at least 6 points, at least 7 points, at least 8 points, at least 9 points, at least 10 points, at least 11 points, at least 12 points, at least 13 points, at least 14 points, at least 15 points, at least 16 points, at least 17 points, at least 18 points, at least 19 points, at least 20 points, at least 21 points, at least 22 points, at least 23 points, or at least 24 points after treatment with eculizumab. In some embodiments, the MG-ADL score of the patient is reduced by at least 1 point after a course of treatment with eculizumab. In some embodiments, the MG-ADL of the patient is reduced by 1, 2, 3, 4, 5, 6, 7, 8, 9, 10, 11, 12, 13, 14, 15, 16, 17, 18, 19, 20, 21, 22, 23, or 24 points after a course of treatment with eculizumab.

According to some embodiments, the course of treatment with eculizumab lasts for 26 weeks. According to some embodiments, the course of treatment lasts for 26-52, 26-78, 26-104, 26-130, 26-156, 26-182, 26-208 weeks, or more. In some embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182 weeks.

According to some embodiments, the course of treatment lasts for greater than 1, 2, 3, 4, 5, 10, 15, 20, 25, 30, 35, 40, 45, 50, 55, 60, 65, 70, 75, 80, or more years. In some embodiments, the course of treatment lasts for the remainder of the subject's life.

According to some embodiments, during the course of treatment, one or more symptoms or scores associated with MG improves during the course of treatment and is maintained at the improved level throughout treatment. For example, MG-ADL can improve after 26 weeks of treatment with a therapeutic antibody that specifically binds C5 and then remain at the improved level for the duration of the treatment, which is 52 weeks of treatment with a therapeutic antibody that specifically binds C5. One example of a therapeutic antibody that binds C5 is eculizumab.

In some embodiments, the first sign of improvement occurs by 26 weeks of treatment with a therapeutic antibody that specifically binds C5. According to some embodiments, the first sign of improvement occurs between weeks 1-26, 26-52, 52-78, 78-104, 104-130, 130-156, 156-182, or 182-208 of treatment with a therapeutic antibody that specifically binds C5. In some embodiments, the first sign of improvement occurs at week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182.

According to some embodiments, the first sign of improvement is maintained for a number of weeks during treatment with a binding protein that specifically binds C5, such as eculizumab or an eculizumab variant such as BNJ441. According to some embodiments, this number of weeks is at least 26. According to some embodiments, this number of weeks is 1-26, 26-52, 52-78, 78-104, 104-130, 130-156, 156-182, or 182-208. In some embodiments, this number of weeks is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182.

According to some embodiments, eculizumab or other anti-C5 antibodies such as BNJ441, BNJ421, 7086, and 8110 can be administered to a subject suffering from MG at between 300 mg to 1200 mg. According to some embodiments, the induction dose of eculizumab or other anti-C5 antibodies such as BNJ441, BNJ421, 7086, and 8110 is between 300 mg to 1200 mg. According to some embodiments, the maintenance dose of eculizumab or other anti-C5 antibodies such as BNJ441, BNJ 421, 7086, and 8110 is about 300, 600, 900 or 1200 mg.

These doses can be administered once a month, once every two weeks, once a week, twice a week, or daily. According to some embodiments, the dose is administered once every two weeks or once a week. According to some embodiments, eculizumab is administered to a subject suffering from MG in a multiphase dosing regimen. According to some embodiments, the multiphase dosing regimen has 2, 3, 4, 6, 7, 8, 9, 10, or more phases. In some embodiments, each phase provides a higher dose than the phase before it.

In some embodiments, the eculizumab multiphase dosing regimen has two phases. The first phase is an induction phase. This phase provides a dose of 300, 600, 900 or 1200 mg per week. In some embodiments, this phase lasts for 2, 3, 4, 5, 6, 7, 8, 9, or 10 weeks. In some embodiments, this phase lasts between 2 and 6 weeks. In other embodiments, the phase lasts for 5 weeks. According to some embodiments, the dose given any week is higher than the previous week. In some embodiments, the dose remains the same for a number of weeks and is then increased. In some embodiments, the dose remains the same for the first 1, 2, 3, 4, 5, 6, 7, 8, or 9 weeks and is then increased. In some embodiments, the dose remains the same for the first 4 weeks.

According to some embodiments, the second phase of eculizumab dosing is the maintenance phase. The maintenance phase of eculizumab dosing can last for between 6 weeks and the life of the subject. According to some embodiments, the maintenance phase lasts for 26-52, 26-78, 26-104, 26-130, 26-156, 26-182, 26-208 weeks, or more. In some embodiments, the maintenance phase lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182 weeks. According to some embodiments, the maintenance phase lasts for greater than 1, 2, 3, 4, 5, 10, 15, 20, 25, 30, 35, 40, 45, 50, 55, 60, 65, 70, 75, 80 years, or more years. In some embodiments, the maintenance phase lasts for the remainder of the subject's life.

In some embodiments, the eculizumab multiphase dosing regimen includes a third phase. This third phase is used when an MG patient must undergo a rescue procedure to maintain clinical stability and includes administering plasma exchange and/or dosing with IVIg. In this phase after plasma is exchanged a dose of eculizumab is administered to replace the drug lost in plasma exchange. According to some embodiments, this post-rescue eculizumab dose is between 300 mg to 1200 mg.

### 2. Pharmaceutical Compositions

Pharmaceutical compositions comprising eculizumab, either alone or in combination with prophylactic agents, therapeutic agents, and/or pharmaceutically acceptable carriers are provided. The pharmaceutical compositions comprising eculizumab provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art.

An exemplary, non-limiting range for a therapeutically or prophylactically effective amount of eculizumab or other anti-C5 antibodies such as BNJ441, BNJ 421, 7086, and 8110 provided herein is 300 mg to 1200 mg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed methods.

### 3. Combination Therapy

An anti-C5 antibody provided herein also can also be administered with one or more additional medicaments or therapeutic agents useful in the treatment of MG. For example, the additional agent can be a therapeutic agent art-recognized as being useful to treat myasthenia gravis or condition being treated by the antibody provided herein. The combination can also include more than one additional agent, e.g., two or three additional agents.

The binding agent in some embodiments is administered with an agent that is a protein, a peptide, a carbohydrate, a drug, a small molecule, or a genetic material (e.g., DNA or RNA). In some embodiments, the agent is one or more cholinesterase inhibitors, one or more corticosteroids, and/or one or more immunosuppressive drugs (most commonly azathioprine [AZA], cyclosporine, and/or mycophenolate mofetil [MMF]).

### EXAMPLES

### Example 1: Effectiveness of Eculizumab in Treating Myasthenia Gravis in Pediatric Human Subjects

Described herein is an open-label, multicenter study to evaluate the efficacy, safety, pharmacokinetics, and pharmacodynamics of eculizumab in pediatric patients with refractory generalized myasthenia gravis. The purpose of this study is to evaluate the efficacy, safety, pharmacokinetics, and pharmacodynamics of eculizumab in the treatment of pediatric refractory gMG based on change from baseline in the quantitative Myasthenia Gravis (QMG) score for disease severity. The study will consist of an up to 4-week Screening Period, 26-week Primary Evaluation Treatment Period, an additional (up to) to 208-week Extension Period, and an 8-week Safety Follow-up Period.

The study design will include the following criteria:

Study Type: Interventional

Primary Purpose: Treatment

Study Phase: Phase 3

Interventional Study Model: Single Group Assignment

Number of Arms: 1

Masking: None (Open Label)

Allocation: N/A

Enrollment: ≥12

### Arms and Interventions

| Arms | Assigned Interventions |
|---|---|
| Experimental: Eculizumab Intravenous | Drug: Eculizumab |
| (IV) Infusion | Eculizumab will be administered |
| In the Primary Evaluation Treatment | by IV infusion. |
| Period (26 weeks), eculizumab will be | |
| administered weekly during the initial | |
| induction phase and every 2 weeks | |
| during the maintenance phase. | |
| In the Extension Period (up to 208 | |
| weeks), participants will continue to | |
| receive eculizumab every 2 weeks. | |
| Eculizumab will be administered at | |
| doses of 300, 600, 900, or 1200 | |
| milligrams (mg), based on the | |
| participant's current body weight. | |

### Objectives:

The primary objective of this study is to evaluate the efficacy of eculizumab in the treatment of pediatric refractory generalized myasthenia gravis (gMG) based on change from Baseline in the Quantitative Myasthenia Gravis score for disease severity (QMG).
The secondary objectives of the study are to:
Evaluate the safety and tolerability of eculizumab in the treatment of pediatric refractory gMG
Evaluate the efficacy of eculizumab in the treatment of pediatric refractory gMG based on change from Baseline in the following measures:
   Myasthenia Gravis Activities of Daily Living profile (MG-ADL)
   Myasthenia Gravis Composite score (MGC)
Evaluate the effect of eculizumab on the following quality of life measures:
   European Quality of Life 5-Dimension Youth (EQ-5D-Y) Questionnaire—EQ-5D-Y Proxy version for patients <8 years of age or EQ-5D-Y version for patients ≥8 years of age
   Neurological Quality of Life Pediatric Fatigue (Neuro-QoL Pediatric Fatigue) Questionnaire—Neuro-QoL Pediatric Proxy version for patients <8 years of age or Neuro-QoL Pediatric Fatigue for patients ≥ 8 years of age
Evaluate MGFA Post-Interventional Status over time regardless of rescue treatment
Describe the total number and percentage of patients with clinical deteriorations, myasthenic crises, and rescue therapy use over time
Describe the pharmacokinetics (PK) and pharmacodynamics (PD) of eculizumab treatment in pediatric refractory gMG patients to confirm the pediatric dosing regimen selected through modeling and simulation following 26 weeks of eculizumab treatment
   The Extension Period objectives are to:
Characterize long-term safety beyond 26 weeks of eculizumab treatment in pediatric patients with refractory gMG
Characterize long-term efficacy beyond 26 weeks of eculizumab treatment in pediatric patients with refractory gMG

### Endpoints:

### Primary Efficacy Endpoint:

Change from Baseline in the QMG total score over time regardless of rescue treatment. Secondary Efficacy Endpoints:
Change from Baseline in the MG-ADL total score over time regardless of rescue treatment
Proportion of patients with ≥3-point reduction in the MG-ADL total score over time with no rescue treatment
Proportion of patients with ≥3-point reduction in the MG-ADL total score over time regardless of rescue treatment
Proportion of patients with ≥5-point reduction in the QMG total score over time with no rescue treatment
Proportion of patients with ≥5-point reduction in the QMG total score over time regardless of rescue treatment
Change from Baseline in the MGC total score over time regardless of rescue treatment
Change from Baseline in EQ-5D-Y over time regardless of rescue treatment
Change from Baseline in Neuro-QoL Pediatric Fatigue over time regardless of rescue treatment
MGFA Post-Interventional Status over time regardless of rescue treatment
Total number and percentage of patients with clinical deteriorations, myasthenic crises, and rescue therapy use over time
Extension Period Efficacy Endpoints:
Total number and percentage of patients with clinical deteriorations and/or myasthenic crises during the study
Total number and percentage of patients needing rescue therapy during the study
Change from Baseline in the QMG total score regardless of rescue treatment
Change from Baseline in the MG-ADL total score regardless of rescue treatment
Change from Baseline in the MGC total score regardless of rescue treatment
Change from Baseline in Neuro-QoL Pediatric Fatigue regardless of rescue treatment
Change from Baseline in EQ-5D-Y regardless of rescue treatment
Change from Baseline in MGFA Post-Interventional Status regardless of rescue treatment
Safety Endpoints:
Frequency of adverse events (AEs) and serious adverse events (SAEs)
Frequency of adverse events leading to discontinuation
Incidence of antidrug antibodies (ADA)
Changes from Baseline in vital signs
Change from Baseline in electrocardiogram parameters
Change from Baseline in laboratory assessments

### Pharmacokinetic and Pharmacodynamic Endpoints:

Pharmacokinetic/PD parameters including maximum plasma drug concentration (Cₘₐₓ), terminal half-life (t_{1/2}), trough (C_{trough}), clearance, free complement protein 5 (C5), and in vitro hemolytic assay; assessed at Baseline and various time points including 24 hours (Day 2), Week 12, and Week 26 during treatment
   Diagnosis and Main Criteria for Patient inclusion/Exclusion:

### Inclusion Criteria:

1. Male or female pediatric patients 6 to <18 years of age at time of assent/consent.
2. Patient's legal guardian must be willing and able to give written informed permission and the patient must be willing to give written informed assent (if applicable as determined by the central or local Institutional Review Board [IRB]/Institutional [or Independent] Ethics Committee [IEC]) and comply with the study visit schedule.
3. Parent or other legal guardian must be willing to comply with study requirements for the duration of the study.
4. Must be vaccinated against N meningitidis if not already vaccinated within the time period of active coverage specified by the vaccine manufacturer, or vaccinated according to current medical/country guidelines at least 2 weeks prior to receiving the first dose of study drug. Patients who require vaccination against N meningitidis will be vaccinated according to current medical/country guidelines; if the vaccine is given less than 2 weeks prior to the first dose of study drug, the patient must receive appropriate prophylactic antibiotics until 2 weeks after the vaccination. Patients who cannot be vaccinated must receive antibiotic prophylaxis for the entire treatment period and for 5 months following the last dose of eculizumab.
5. Documented vaccination against H influenzae and S pneumoniae infections prior to dosing as per local and country specific immunization guidelines for the appropriate age group.
6. Diagnosis of MG confirmed by positive serologic test for anti-AChR-Ab at Screening, and one of the following:
   a. History of abnormal neuromuscular transmission test demonstrated by single-fiber electromyography or repetitive nerve stimulation, or
   b. History of positive anticholinesterase test (e.g., edrophonium chloride or neostigmine test), or
   c. Patient demonstrated improvement in MG signs on oral AChIs, as assessed by the Investigator.
7. Presence of refractory gMG, defined as patients with gMG who have one or more of the following:
   a. Failed treatment ≥1 year with at least 1 IST, defined as:
      1) Persistent weakness with impairment of activities of daily living, or
      2) Myasthenia gravis exacerbation and/or crisis while on treatment, or
      3) Intolerance to ISTs due to side effect or comorbid condition(s). Immunosuppressants include, but are not limited to, corticosteroids, azathioprine (AZA), mycophenolate mofetil (MMF), methotrexate (MTX), cyclosporine, tacrolimus, or cyclophosphamide.
   b. Require maintenance PE or IVIg to control symptoms (i.e., patients who require PE or IVIg on a regular basis for the management of muscle weakness at least every 3 months over the last 12 months prior to Screening).
   c. In the opinion of the Investigator, MG poses a significant functional burden despite treatment.
8. Myasthenia Gravis Foundation of America (MGFA) Clinical Classification of Class II to IV at Screening.
9. QMG total score ≥12 at Screening.
10. All MG-specific treatment is on a stable dosing regimen of adequate duration prior to Screening as follows:
a. If patients who enter the study are receiving AZA, they must have been on AZA for ≥6 months and have been on a stable dose for ≥2 months prior to Screening.
   b. If patients who enter the study are receiving other ISTs (i.e., MMF, MTX, cyclosporine, tacrolimus, cyclophosphamide), they must have been on the IST for ≥3 months and have been on a stable dose for ≥4 weeks prior to Screening.
   c. If patients who enter the study are receiving maintenance IVIg at Screening, they must have been on maintenance IVIg for at least 12 months and on a stable dose for ≥3 months prior to Screening, with the frequency and dose expected to remain stable during Screening and for 12 weeks following the first dose of study drug. Note: A maximum of 4 patients on maintenance IVIg aged 12 to <18 years are eligible to be enrolled in the study. All other patients aged 12 to <18 years must not have received maintenance IVIg within 3 months of Screening.
   d. If patients who enter the study are receiving oral corticosteroids, they must have been on a stable dose for ≥4 weeks prior to Screening.
   e. If patients who enter the study are receiving a cholinesterase inhibitor, they must have been on a stable dose for ≥2 weeks prior to Screening.
11. Female patients of childbearing potential (i.e., have achieved menarche) and male patients with female partners of childbearing potential must follow protocol-specified guidance for avoiding pregnancy while on treatment and for 5 months after the last dose of study drug.
12. Male patients with a female spouse/partner of childbearing potential or a pregnant or breastfeeding spouse or partner must agree to use double barrier contraception (male condom plus appropriate barrier method for the female partner) while on treatment and for at least 5 months after the last dose of study drug.

### Exclusion Criteria:

1. History of thymoma or other neoplasms of the thymus.
2. History of thymectomy within 12 months prior to Screening.
3. Weakness only affecting ocular or periocular muscles (MGFA Class I).
4. Myasthenia Gravis crisis or impending crisis at or during Screening (MGFA Class V).
5. Are pregnant or lactating.
6. Any unresolved acute, or chronic, systemic bacterial or other infection, which is clinically significant in the opinion of the Investigator and has not been treated with appropriate antibiotics.
7. Unresolved meningococcal infection.
8. Use of PE within 4 weeks prior to first dose.
9. Use of rituximab within 6 months prior to first dose.
10. Participation in another interventional treatment study or use of any experimental therapy within 30 days before initiation of study drug on Day 1 in this study or within 5 half-lives of that investigational product, whichever is greater.
11. Have previously received treatment with eculizumab or other complement inhibitors.
12. Hypersensitivity to murine proteins or to one of the excipients of eculizumab.
13. Any medical or psychological condition that, in the opinion of the Investigator, might interfere with the patient's participation in the study, poses any added risk for the patient, or confounds the assessment of the patient.
14. Known or suspected history of drug or alcohol abuse or dependence within 1 year prior to the start of Screening.

### 1. Investigational Plan

### 1.1. Overall Trial Design and Plan

Described herein is an open-label, multicenter study (i.e., ECU-MG-303) to evaluate the efficacy, safety, PK, and PD of intravenous eculizumab in pediatric patients (n≥12) aged 6 to <18 years with acetylcholine receptor (AChR)-antibody (Ab) positive refractory gMG. There are 4 periods in this study: Screening Period (2 to 4 weeks), Primary Evaluation Treatment Period (26 weeks), Extension Period (up to an additional 208 weeks), and Follow-up Period (8 weeks). All patients who completed Week 26 of Study ECU-MG-303 continued receiving eculizumab in the Extension Period of this study for up to an additional 208 weeks. The 8-week Follow-up Period is required following the last dose of study drug for all patients upon withdrawal or discontinuation from the study or upon completion of the study when the patient is not continuing to receive eculizumab treatment.

Patients continued to receive acetylcholinesterase inhibitors (AChI), intravenous immunoglobulins (IVIg), and immunosuppressive therapies (ISTs) during the study where applicable under certain restrictions. For patients who entered the study receiving any background therapy, the dose and frequency was not changed during the Primary Evaluation Treatment Period before Week 12, unless deemed necessary per the Investigator based on clinical safety evaluation and Sponsor approval was obtained. Dose change with background medication was permitted after Week 12 at the Investigator's discretion and with Sponsor notification. During the Extension Period, changes in background medications were permitted at the Investigator's discretion and with Sponsor notification.

If a patient withdrew from the study or discontinued eculizumab treatment at any time, the patient was required to complete an Early Termination (ET) visit at the time of withdrawal and a Follow-up visit 8 weeks following the last dose of study drug. The overall study duration for an individual patient can be up to 246 weeks (approximately 4.7 years) from the Screening Period through the Follow-up Period.

This pediatric study was designed to assess the efficacy and safety of eculizumab in AChR-Ab positive refractory pediatric gMG patients. All patients may continue to receive ISTs during the study. The number of eligible refractory gMG patients aged 12 to <18 years entering on maintenance IVIg therapy was capped at 4 patients. There was no limit on the number of patients aged 6 to <12 years who may enter the study on maintenance IVIg. This change was made based on the higher prevalence of maintenance IVIg use in children.

### 1.1.1. Screening Period (2-4 Weeks)

At the screening visit, after obtaining the informed consent of the parent or other legal guardian, and the patient's informed assent, when applicable, the subject was screened for trial eligibility through medical history review, demographic data, and laboratory assessments. Assessments included confirmation of a refractory gMG diagnosis per protocol-defined inclusion/exclusion criteria, QMG total score, history of previous MG treatments and therapies, history of MG exacerbation or crisis and the treatment for each exacerbation/crisis, and a comprehensive review of medical history, including vaccination history, as well as any non-MG comorbid conditions. When an eligible patient met all inclusion criteria, but none of the exclusion criteria, the Principal Investigator notified the Sponsor to obtain Medical Monitor approval prior to enrolling the patient.

If all inclusion criteria and none of the exclusion criteria were met, subjects were vaccinated against N. meningitidis, if not already vaccinated within the time period of active coverage specified by the vaccine manufacturer or vaccinate according to current medical/country guidelines. If the vaccine was administered within 2 weeks of the first dose of study drug, patients received appropriate prophylactic antibiotics until 2 weeks after vaccination. Patients remained within the vaccine manufacturer's specified period of active coverage during study participation and for 5 months following the last dose of eculizumab. In addition to meningococcal vaccination, patients were vaccinated against Haemophilus influenzae (H influenzae) and Streptococcus pneumoniae (S pneumoniae), if not already vaccinated within the time period of active coverage specified by the vaccine manufacturer, and strictly adhere to the national vaccination recommendations for each age group.

The site notified the Sponsor if any patient experienced signs and symptoms of MG worsening that require rescue or foreseeable imminent change to the background medication during the Screening Period. Following discussion with the Sponsor, a decision was made about whether the patient may be enrolled in the study, be withdrawn and, possibly, rescreened at a later date. Patients whose MG was unstable (as determined by the Investigator) during the Screening Period may be rescreened based on discussion and agreement between the Investigator and the Study Medical Monitor.

### 1.1.2. Primary Evaluation Treatment Period (26 Weeks)

All subjects received eculizumab by IV infusion during the open-label Primary Evaluation Treatment Period. Dosing was initiated with a weekly weight-based induction regimen and, thereafter, every 2 weeks. Weight may change for an individual patient during the study, and dosing was based on the most recently recorded body weight at a prior dosing visit.

Sites informed patients and their parent or legal guardian of potential signs and symptoms of MG worsening or clinical deterioration, including myasthenic crisis, and instructed them to contact the Investigator in the event of symptoms. The Investigator made every effort to evaluate a patient reporting worsening signs and symptoms of MG as soon as possible and within 48 hours of notification. The Investigator assessed for clinical deterioration and treat the patient accordingly.

### 1.1.3. Extension Period (Up to an Additional 208 Weeks)

After completing the 26-week Primary Evaluation Treatment Period, subjects may be provided an opportunity to enter an extension trial of this study for up to an additional 208 weeks. Weight may change for an individual patient during the study, and dosing should be based on the most recently recorded body weight at a prior dosing visit. Patients may have an opportunity to receive study drug administration remotely at a medical facility that is located near the patient's home or at the patient's home with the permission of the Principal Investigator in accordance with all national, state, and local laws or regulations of the pertinent regulatory authorities.

1.1.4. Follow-up Period (8 Weeks Post-Treatment)

If a subject withdrew or is discontinued from this trial at any time after receiving any amount of eculizumab or did not wish to enter the extension trial after completion of this trial, the subject was required to complete both an ET Visit at the time of withdrawal and a Follow-up Visit at 8 weeks following the last eculizumab dose. Adverse events leading to patient discontinuation from the study are followed until resolution or are medically stable in the opinion of the Investigator.

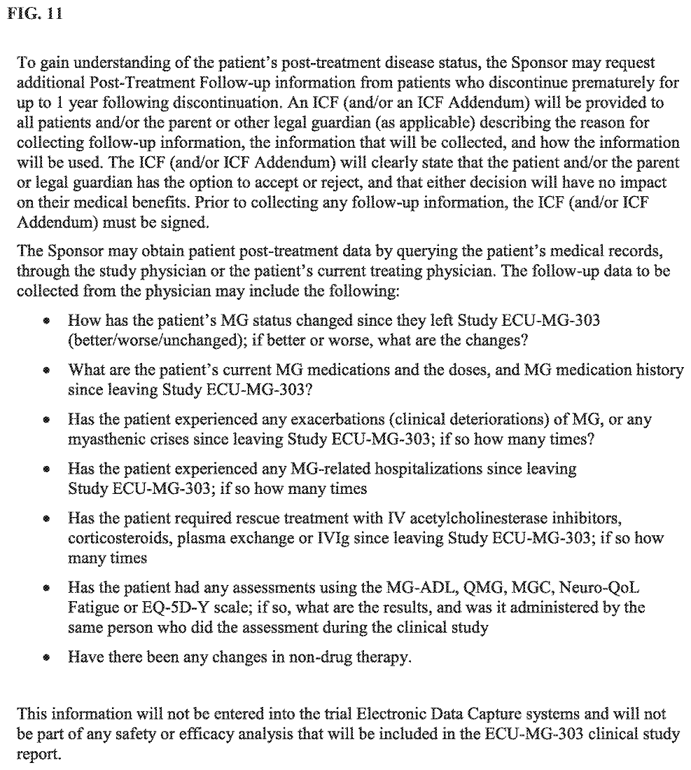

Patients who completed the study and transitioned to uninterrupted treatment with commercially available eculizumab were not required to complete a follow-up visit. The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon thereafter as feasible. The Sponsor may seek to collect follow-up information concerning MG status in patients, post-treatment for up to 1 year from the end-of-study (EOS)/ET Visit (FIG. 11).

### 1.1.5. Unscheduled Visits

Additional (Unscheduled) visits outside the specified visits for study procedures, tests, and assessments may be performed at the request of the Investigator or Sponsor. If an Unscheduled Visit is performed, any tests, procedures, or assessments performed at the Unscheduled Visits must be recorded on the electronic case report forms (eCRFs).

### 1.2. Study Flow Diagram and Schedule of Assessments

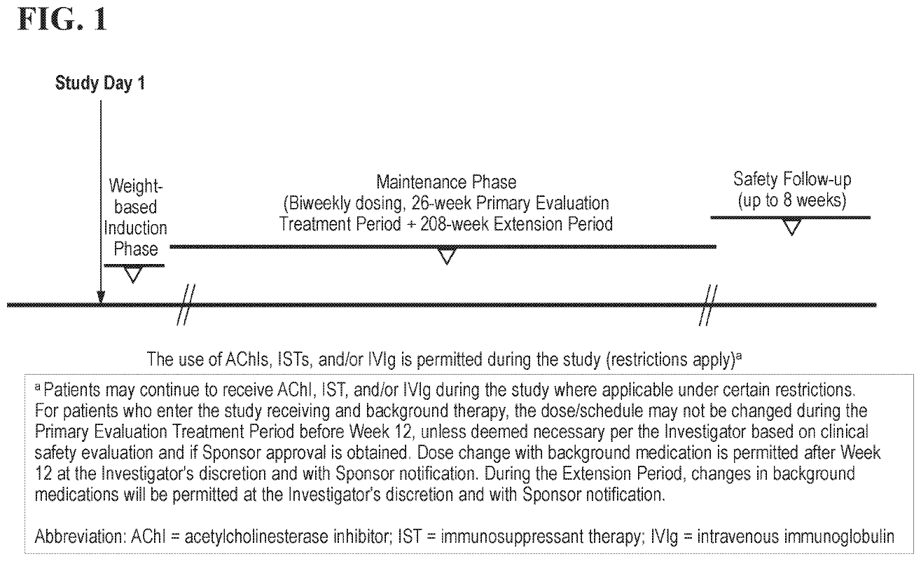

The flow diagram for the study design is illustrated in FIG. 1. The Schedule of Assessments during the study for patients in weight cohorts ≥40 kg, 30 to <40 kg and 20 to <30 kg are summarized in Table 6 and Table 7. The Schedule of Assessments for patients in weight cohort 10 to <20 kg are summarized in Table 8 and Table 9. Weight cohort may change for an individual patient during the study and is based on the most recently recorded body weight at a prior dosing visit.

TABLE 6
SCHEDULE OF ASSESSMENTS PART I: WEIGHT COHORTS ≥40
KG, 30 TO <40 KG, AND 20 TO <30 KG

| | Period/Phase | | | | | | | | | | |
|---|---|---|---|---|---|---|---|---|---|---|---|
| | Screening | Primary Evaluation Treatment Period | | | | | | | | | |
| | Visit Location^{a} | | | | | | | | | | |
| | In Clinic | In Clinic | | | | | | | | | |
| | Study Visit^{b} | | | | | | | | | | |
| | Screening | | | | | | | | | | |
| | Visit | 2 | 3 | 4 | 5 | 6 | 7 | 8 | 9 | 10 | 11 |
| Study Week | −2 to −4 | D 1 | W 1 | W 2 | W 3 | W 4 | W 6 | W 8 | W 10 | W 12 | W 14 |
| | weeks | | | | | | | | | | |
| Window (Days) | | | ±2 | | | | | | | | |
| Informed Consent | X | | | | | | | | | | |
| Medical History | X | | | | | | | | | | |
| MG History | X | | | | | | | | | | |
| MGFA Clinical | X | | | | | | | | | | |
| Classification | | | | | | | | | | | |
| Weight^{e,f} | X | X | X | X | X | X | X | X | X | X | X |
| Height^{g} | X | X | | | | | | | | X | |
| Vital Signs | X | X | X | X | X | X | X | X | X | X | X |
| Physical Exam | X | | | | | | | | | X | |
| 12-Lead ECG | X | X | | | | | | | | X | |
| Concomitant | X | X | X | X | X | X | X | X | X | X | X |
| Medication | | | | | | | | | | | |
| Adverse Event^{h} | X^{h} | X | X | X | X | X | X | X | X | X | X |
| MG-ADL^{i,j} | X^{k} | X | X | X | X | X | | X | | X | |
| QMG^{i,k,l} | X^{k} | X | X | X | X | X | | X | | X | |
| MGC^{i,l} | X^{k} | X | X | X | X | X | | X | | X | |
| Neuro-QoL Pediatric | | X | | | | X | | X | | X | |
| Fatigue | | | | | | | | | | | |
| EQ-5D-Y | | X | | | | X | | X | | X | |
| MGFA-PISⁱ | | | | | | X | | | | X | |
| MGFA-Therapy Status | X | X | | | | | | | | X | |
| AChR Ab | X | | | | | | | | | | |
| Clinical Laboratory | X | X | | | | | | | | X | |
| Tests | | | | | | | | | | | |
| Serum Pregnancy Test^{m} | X | | | | | | | | | | |
| Urine Pregnancy Testⁿ | | X | | | | X | | X | | X | |
| PK, Hemolysis, Free | | B/P | T/P | | | T/P | | | | T/P | |
| C5^{o} | | 24 h | | | | | | | | | |
| ADA^{o} | | B | | | | | | | | X | |
| Medically Indicated | | | | | | | | | | | |
| Tests^{p} | | | | | | | | | | | |
| N meningitidis | X | | | | | | | | | | |
| Vaccination^{s} | | | | | | | | | | | |
| H influenzae | X | | | | | | | | | | |
| Vaccination^{r} | | | | | | | | | | | |
| S pneumonia | X | | | | | | | | | | |
| Vaccination^{r} | | | | | | | | | | | |
| Check for | | X | X | X | X | X | X | X | X | X | X |
| Revaccination Status^{s} | | | | | | | | | | | |
| Patient Safety | | X | X | X | X | X | X | X | X | X | X |
| Information Card^{t} | | | | | | | | | | | |
| Study Drug | | 900 | 900 | 900 | 900 | 1200 | 1200 | 1200 | 1200 | 1200 | 1200 |
| Infusion ≥40 kg^{f,u,v} | | mg | mg | mg | mg | mg | mg | mg | mg | mg | mg |
| Study Drug | | 600 | 600 | 900 | N/A | 900 | 900 | 900 | 900 | 900 | 900 |
| Infusion 30 | | mg | mg | mg | | mg | mg | mg | mg | mg | mg |
| to <40 kg^{f,u,v} | | | | | | | | | | | |
| Study Drug | | 600 | 600 | 600 | N/A | 600 | 600 | 600 | 600 | 600 | 600 |
| Infusion 20 | | mg | mg | mg | | mg | mg | mg | mg | mg | mg |
| to <30 kg^{f,u,v} | | | | | | | | | | | |

| | Period/Phase | | | | | | | | |
|---|---|---|---|---|---|---|---|---|---|
| | Primary Evaluation Treatment Period | | | | | | | | |
| | Visit Location^{a} | | | | | | | | |
| | In Clinic | | | | | | In Clinic | | |
| | Study Visit^{b} | | | | | | | | |
| | 12 | 13 | 14 | 15 | 16 | 17 | CD^{c} | ET/EOS | F/U^{d} |
| Study Week | W 16 | W 18 | W 20 | W 22 | W 24 | W 26 | | | +W 8 |
| Window (Days) | ±2 | | | | | | | | ±2 |
| Informed Consent | | | | | | | | | |
| Medical History | | | | | | | | | |
| MG History | | | | | | | | | |
| MGFA Clinical | | | | | | | | | |
| Classification | | | | | | | | | |
| Weight^{e,f} | X | X | X | X | X | X | X | X | X |
| Height^{g} | | | | | | X | | X | |
| Vital Signs | X | X | X | X | X | X | X | X | X |
| Physical Exam | | | | | | X | | X | |
| 12-Lead ECG | | | | | | X | | X | |
| Concomitant | X | X | X | X | X | X | X | X | X |
| Medication | | | | | | | | | |
| Adverse Event^{h} | X | X | X | X | X | X | X | X | X |
| MG-ADL^{i,j} | X | | X | | | X | X | X | X |
| QMG^{i,k,l} | X | | X | | | X | X | X | X |
| MGC^{i,l} | X | | X | | | X | X | X | X |
| Neuro-QoL Pediatric | X | | X | | | X | | X | |
| Fatigue | | | | | | | | | |
| EQ-5D-Y | X | | X | | | X | | X | |
| MGFA-PISⁱ | | | | | | X | | X | X |
| MGFA-Therapy Status | | | | | | X | | | |
| AChR Ab | | | | | | | | | |
| Clinical Laboratory | | | | | | X | X | X | |
| Tests | | | | | | | | | |
| Serum Pregnancy Test^{m} | | | | | | X | | X | |
| Urine Pregnancy Testⁿ | X | | X | | X | | | X | X |
| PK, Hemolysis, Free | | | | | | T/P | T | T | |
| C5^{o} | | | | | | | | | |
| ADA^{o} | | | | | | X | X | X | |
| Medically Indicated | | | | | | | X | | |
| Tests^{p} | | | | | | | | | |
| N meningitidis | | | | | | | | | |
| Vaccination^{s} | | | | | | | | | |
| H influenzae | | | | | | | | | |
| Vaccination^{r} | | | | | | | | | |
| S pneumonia | | | | | | | | | |
| Vaccination^{r} | | | | | | | | | |
| Check for | X | X | X | X | X | X | X | X | X |
| Revaccination Status^{s} | | | | | | | | | |
| Patient Safety | X | X | X | X | X | X | X | X | X |
| Information Card^{t} | | | | | | | | | |
| Study Drug | 1200 | 1200 | 1200 | 1200 | 1200 | 1200 | | | |
| Infusion ≥40 kg^{f,u,v} | mg | mg | mg | mg | mg | mg | | | |
| Study Drug | 900 | 900 | 900 | 900 | 900 | 900 | | | |
| Infusion 30 | mg | mg | mg | mg | mg | mg | | | |
| to <40 kg^{f,u,v} | | | | | | | | | |
| Study Drug | 600 | 600 | 600 | 600 | 600 | 600 | | | |
| Infusion 20 | mg | mg | mg | mg | mg | mg | | | |
| to <30 kg^{f,u,v} | | | | | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Unscheduled visits and procedures will be performed at the Investigator's discretion, and results will be recorded in the eCRF.
^{c}Perform an evaluation visit for CD as soon as possible within 48 hours of notification of symptom onset. The PI may schedule additional evaluation visits at their discretion.
^{d}Patients who complete the study and transition to uninterrupted treatment with commercially available eculizumab will not be required to complete a follow-up visit.
^{e}Collect weight with minimal clothing.
^{f}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{g}Collect height with no shoes or footwear.
^{h}Serious adverse events are recorded from time of signed consent, with nonserious adverse events recorded from the time of first dose.
ⁱPrior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{j}The recall period for the MG-ADL is the preceding 7 days, or since the last visit if the visit interval is <7 days
^{k}Recommendation to perform QMG assessment first to confirm eligibility. MG-ADL and MGC assessments only necessary once QMG eligibility is confirmed.
^{l}Withhold acetylcholinesterase inhibitors for at least 10 hours prior to the QMG and MGC tests.
^{m}Perform serum pregnancy tests on all female patients of childbearing potential at the specified time points. Additional pregnancy tests (serum or urine) may also be performed at any visit at the PI's discretion.
ⁿPerform urine pregnancy tests on all female patients of childbearing potential at the specified time points. Additional pregnancy tests (serum or urine) may also be performed at any visit at the PI's discretion.
^{o}Obtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion. Obtain 24-hour post-dose blood samples for PK, hemolysis, and free C5 after study drug infusion at Visit 1 (Day 1).
^{p}Any test(s) medically indicated may be performed at the discretion of the Investigator.
^{q}Patients must be vaccinated against N meningitidis if not already vaccinated within the time period of active coverage specified by the vaccine manufacturer, or vaccinate according to current medical/country guidelines at least 2 weeks prior to receiving the first dose of study drug. Patients who require vaccination against N meningitidis will be vaccinated according to current medical/country guidelines and must receive appropriate prophylactic antibiotics until 2 weeks after the vaccination if the vaccine is given less than 2 weeks prior to the first dose of study drug. Patients must remain within the vaccine manufacturer's specified period of active coverage during study participation. Patients who cannot be vaccinated must receive antibiotic prophylaxis for the entire treatment period and for 5 months following the last dose of eculizumab. Any additional vaccinations may also be performed in accordance with local guidelines.
^{r}Vaccinate patients against H influenzae and S pneumoniae, if not already vaccinated, prior to receiving the first eculizumab infusion.
^{s}Patients must be revaccinated for N meningitidis, H influenza, and S pneumoniae to provide active coverage as specified by the vaccine manufacturer or according to current medical/country guidelines.
^{t}Review the Patient Safety Information Card with the patient and caregiver to discuss the importance of carrying the safety card at all times.
^{u}Administer study drug after completion of all other tests and procedures, excluding the peak blood sampling for PK, hemolysis, and free C5 assay.
^{v}For supplemental weight-based eculizumab dosing, refer to Table 19 and Table 20.
Abbreviations:
AChR Ab = acetylcholine receptor antibody;
ADA = anti-drug antibodies;
B = baseline sample;
C5 = complement protein 5;
CD = clinical deterioration;
D = day;
ECG = electrocardiogram;
eCRF = electronic case report form;
EOS = End of Study;
EQ-5D-Y = European Quality of Life 5-Dimension Youth;
ET = Early Termination;
F/U = Follow-up;
MG = myasthenia gravis;
MG-ADL = Myasthenia Gravis Activities of Daily Living profile;
MGC = Myasthenia Gravis Composite score;
MGFA = Myasthenia Gravis Foundation of America;
MGFA-PIS = Myasthenia Gravis Foundation of America Post-Intervention Status;
N/A = not applicable;
Neuro-QoL = Quality of Life in Neurological Disorders;
P = peak sample;
PK = pharmacokinetics;
SCR = Screening;
QMG = Quantitative Myasthenia Gravis score for disease severity;
T = trough sample;
W = week.

TABLE 7
SCHEDULE OF ASSESSMENTS PART II: WEIGHT COHORTS ≥40 KG, 30 TO <40 KG, AND 20 TO <30 KG

| | Period/Phase | | | | | | | | | | | | | | | |
|---|---|---|---|---|---|---|---|---|---|---|---|---|---|---|---|---|
| | Extension Period (Year 1) | | | | | | | | | | | | | | | |
| | Visit Location^{a} | | | | | | | | | | | | | | | |
| | In Clinic/Remote | | | | | | In Clinic | In Clinic/Remote | | | | | In Clinic | In Clinic | | |
| | Study Visit^{b} | | | | | | | | | | | | | | | |
| | 18 | 19 | 20 | 21 | 22 | 23 | 24 | 25 | 26 | 27 | 28 | 29 | 30 | CD^{c} | ET/EOS | F/U^{d} |
| Study Weeks | W 28 | W 30 | W 32 | W 34 | W 36 | W 38 | W 40 | W 42 | W 44 | W 46 | W 48 | W 50 | W 52 | | | +W 8 |
| Window (Days) | ±2 | | | | | | | | | | | | | | | ±2 |
| Weight^{e,f} | X | X | X | X | X | X | X | X | X | X | X | X | X | X | X | X |
| Height^{g} | | | | | | | X | | | | | | X | | X | |
| Vital Signs | X | X | X | X | X | X | X | X | X | X | X | X | X | X | X | X |
| Physical Exam | | | | | | | | | | | | | X | | X | |
| 12-Lead ECG | | | | | | | | | | | | | X | | X | |
| Concomitant | X | X | X | X | X | X | X | X | X | X | X | X | X | X | X | X |
| Medication | | | | | | | | | | | | | | | | |
| Adverse Event^{h} | X | X | X | X | X | X | X | X | X | X | X | X | X | X | X | X |
| MG-ADL^{i,j} | | | | | | | X | | | | | | X | X | X | X |
| QMG^{i,k,l} | | | | | | | X | | | | | | X | X | X | X |
| MGC^{i,k} | | | | | | | X | | | | | | X | X | X | X |
| Neuro-QoL Pediatric | | | | | | | X | | | | | | X | | X | |
| Fatigue | | | | | | | | | | | | | | | | |
| EQ-5D-Y | | | | | | | X | | | | | | X | | X | |
| MGFA-PISⁱ | | | | | | | X | | | | | | X | | X | X |
| Clinical Laboratory | | | | | | | X | | | | | | X | X | X | |
| Tests | | | | | | | | | | | | | | | | |
| Serum Pregnancy Test^{l} | | | | | | | | | | | | | | | X | |
| Urine Pregnancy Test^{m} | X | | X | | X | | X | | X | | X | | X | | X | X |
| PK, Hemolysis, Free | | | | | | | | | | | | | | T | T | |
| C5ⁿ | | | | | | | | | | | | | | | | |
| ADA^{m} | | | | | | | | | | | | | | X | X | |
| Medically Indicated | | | | | | | | | | | | | | X | | |
| Tests^{o} | | | | | | | | | | | | | | | | |
| Check for | X | X | X | X | X | X | X | X | X | X | X | X | X | X | X | X |
| Revaccination Status^{p} | | | | | | | | | | | | | | | | |
| Patient Safety | X | X | X | X | X | X | X | X | X | X | X | X | X | X | X | X |
| Information Card^{q} | | | | | | | | | | | | | | | | |
| Study Drug | 1200 | 1200 | 1200 | 1200 | 1200 | 1200 | 1200 | 1200 | 1200 | 1200 | 1200 | 1200 | 1200 | | | |
| Infusion ≥40 kg^{f,r,s} | mg | mg | mg | mg | mg | mg | mg | mg | mg | mg | mg | mg | mg | | | |
| Study Drug | 900 | 900 | 900 | 900 | 900 | 900 | 900 | 900 | 900 | 900 | 900 | 900 | 900 | | | |
| Infusion 30 | mg | mg | mg | mg | mg | mg | mg | mg | mg | mg | mg | mg | mg | | | |
| to <40 Kg^{f,r,s} | | | | | | | | | | | | | | | | |
| Study Drug | 600 | 600 | 600 | 600 | 600 | 600 | 600 | 600 | 600 | 600 | 600 | 600 | 600 | | | |
| Infusion 20 | mg | mg | mg | mg | mg | mg | mg | mg | mg | mg | mg | mg | mg | | | |
| to <30 Kg^{f,r,s} | | | | | | | | | | | | | | | | |
| Transition follow-up | | | | | | | | | | | | | | | X | |
| call^{t} | | | | | | | | | | | | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Unscheduled visits and procedures will be performed at the Investigator's discretion and results will be recorded in the eCRF.
^{c}Perform an evaluation visit for CD as soon as possible within 48 hours of notification of symptom onset. The PI may schedule additional evaluation visits at their discretion.
^{d}Patients who complete the study and transition to uninterrupted treatment with commercially available eculizumab will not be required to complete a follow-up visit.
^{e}Collect weight with minimal clothing.
^{f}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{g}Collect height with no shoes or footwear.
^{h}Serious adverse events are recorded from time of signed consent, with nonserious adverse events recorded from the time of first dose.
ⁱPrior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{j}The recall period for the MG-ADL is the preceding 7 days, or since the last visit if the visit interval is <7 days.
^{k}Withhold acetylcholinesterase inhibitors for at least 10 hours prior to the QMG and MGC tests.
^{l}Perform serum pregnancy tests on all female patients of childbearing potential at the specified time points. Additional pregnancy tests (serum or urine) may also be performed at any visit at the PI's discretion.
^{m}Perform urine pregnancy tests on all female patients of childbearing potential at the specified time points. Additional pregnancy tests (serum or urine) may also be performed at any visit at the PI's discretion.
ⁿObtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion.
^{o}Any test(s) medically indicated may be performed at the discretion of the Investigator.
^{p}Patients must be revaccinated for N meningitidis, H influenza, and S pneumoniae to provide active coverage as specified by the vaccine manufacturer or according to current medical/country guidelines.
^{q}Review the Patient Safety Information Card with the patient and caregiver to discuss the importance of carrying the safety card at all times.
^{r}Administer study drug after completion of all other tests and procedures, excluding the peak blood sampling for PK, hemolysis, and free C5 assay.
^{s}For supplemental weight-based eculizumab dosing, refer to Table 19 and Table 20.
^{t}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
ADA = anti-drug antibodies;
B = Baseline sample;
C5 = complement protein 5;
CD = clinical deterioration;
ECG = electrocardiogram;
eCRF = electronic case report form;
EOS = End of Study;
EQ-5D-Y = European Quality of Life 5-Dimension Youth;
ET = Early Termination;
F/U = Follow-up;
MG = myasthenia gravis;
MG-ADL = Myasthenia Gravis Activities of Daily Living profile;
MGC = Myasthenia Gravis Composite score;
MGFA = Myasthenia Gravis Foundation of America;
MGFA-PIS = Myasthenia Gravis Foundation of America Post-Intervention Status;
Neuro-QoL = Quality of Life in Neurological Disorders;
P = peak sample;
PK = pharmacokinetics;
SCR = Screening;
QMG = Quantitative Myasthenia Gravis score for disease severity;
T = trough sample;
W = week.

TABLE 8
SCHEDULE OF ASSESSMENTS PART I: WEIGHT COHORT 10 TO <20 KG

| | Period/Phase | | | | | | | | | |
|---|---|---|---|---|---|---|---|---|---|---|
| | Screening | Primary Evaluation Treatment Period | | | | | | | | |
| | Visit Location^{a} | | | | | | | | | |
| | In Clinic | In Clinic | | | | | | | | |
| | Study Visit^{b} | | | | | | | | | |
| | Screening | | | | | | | | | |
| | Visit (1) | 2 | 3 | 4 | 5 | 6 | 7 | 8 | 9 | 10 |
| Study Weeks | −2 to −4 | D 1 | W 1 | W 3 | W 5 | W 7 | W 9 | W 11 | W 13 | W 15 |
| | weeks | | | | | | | | | |
| Window (Days) | | | ±2 | | | | | | | |
| Informed Consent | X | | | | | | | | | |
| Medical History | X | | | | | | | | | |
| MG History | X | | | | | | | | | |
| MGFA Clinical | X | | | | | | | | | |
| Classification | | | | | | | | | | |
| Weight^{e,f} | X | X | X | X | X | X | X | X | X | X |
| Height^{g} | X | X | | | | | | | X | |
| Vital Signs | X | X | X | X | X | X | X | X | X | X |
| Physical Exam | X | | | | | | | | X | |
| 12-Lead ECG | X | X | | | | | | | X | |
| Concomitant | X | X | X | X | X | X | X | X | X | X |
| Medication | | | | | | | | | | |
| Adverse Event^{h} | X^{h} | X | X | X | X | X | X | X | X | X |
| MG-ADL^{i,j} | X^{k} | X | X | X | X | X | | | X | |
| QMG^{i,k,l} | X^{k} | X | X | X | X | X | | | x | |
| MGC^{i,l} | X^{k} | X | X | X | X | X | | | x | |
| Neuro-QoL Pediatric | | X | | | X | | X | | X | |
| Fatigue | | | | | | | | | | |
| EQ-5D-Y | | X | | | X | | X | | X | |
| MGFA-PISⁱ | | | | | X | | | | X | |
| MGFA-Therapy Status | X | X | | | | | | | X | |
| AChR Ab | X | | | | | | | | | |
| Clinical Laboratory | X | X | | | | | | X | | |
| Tests | | | | | | | | | | |
| Serum Pregnancy Test^{m} | X | | | | | | | | | |
| Urine Pregnancy Testⁿ | | X | | | X | | X | | X | |
| PK, Hemolysis, Free | | B/P/ | T/P | | | T/P | | | T/P | |
| C5^{o} | | 24 h | | | | | | | | |
| ADA^{o} | | B | | | | | | | X | |
| Medically Indicated | | | | | | | | | | |
| Tests^{p} | | | | | | | | | | |
| N meningitidis Vaccination^{q}* | X | | | | | | | | | |
| H influenzae Vaccination^{r} | X | | | | | | | | | |
| S pneumonia Vaccination^{r} | X | | | | | | | | | |
| Check for | | X | X | X | X | X | X | X | X | X |
| Revaccination Status^{s} | | | | | | | | | | |
| Patient Safety | | X | X | X | X | X | X | X | X | X |
| Information Card^{t} | | | | | | | | | | |
| Study Drug | | 600 | 300 | 300 | 300 | 300 | 300 | 300 | 300 | 300 |
| Infusion 10 | | mg | mg | mg | mg | mg | mg | mg | mg | mg |
| to <20 kg^{f,u,v,w} | | | | | | | | | | |
| Study Drug | N/A | N/A | 600 | N/A | 600 | 600 | 600 | 600 | 600 | 600 |
| Infusion 20 | | | mg | | mg | mg | mg | mg | mg | mg |
| to <30 kg^{f,u,v,w} | | | | | | | | | | |
| Study Drug | N/A | N/A | 600 | N/A | 900 | 900 | 900 | 900 | 900 | 900 |
| Infusion 30 | | | mg | | mg | mg | mg | mg | mg | mg |
| to <40 kg^{f,u,v,w} | | | | | | | | | | |
| Study Drug | N/A | N/A | 900 | 900 | 1200 | 1200 | 1200 | 1200 | 1200 | 1200 |
| Infusion ≥40 Kg^{f,u,v,w} | | | mg | mg | mg | mg | mg | mg | mg | mg |

| | Period/Phase | | | | | | | | |
|---|---|---|---|---|---|---|---|---|---|
| | Primary Evaluation Treatment Period | | | | | | | | |
| | Visit Location^{a} | | | | | | | | |
| | In Clinic | | | | | | In Clinic | | |
| | Study Visit^{b} | | | | | | | | |
| | 11 | 12 | 13 | 14 | 15 | 16 | CD^{c} | ET/EOS | F/U^{d} |
| Study Weeks | W 17 | W 19 | W 21 | W 23 | W 25 | W 27 | | | +W 8 |
| Window (Days) | ±2 | | | | | | | | ±2 |
| Informed Consent | | | | | | | | | |
| Medical History | | | | | | | | | |
| MG History | | | | | | | | | |
| MGFA Clinical | | | | | | | | | |
| Classification | | | | | | | | | |
| Weight^{e,f} | X | X | X | X | X | X | X | X | X |
| Height^{g} | | | | | | X | | X | |
| Vital Signs | X | X | X | X | X | X | X | X | X |
| Physical Exam | | | | | | X | | X | |
| 12-Lead ECG | | | | | | X | | X | |
| Concomitant | X | X | X | X | X | X | X | X | X |
| Medication | | | | | | | | | |
| Adverse Event^{h} | X | X | X | X | X | X | X | X | X |
| MG-ADL^{i,j} | X | | X | | | X | X | X | X |
| QMG^{i,k,l} | X | | X | | | X | X | X | X |
| MGC^{i,l} | X | | X | | | X | X | X | X |
| Neuro-QoL Pediatric | X | | X | | | X | | X | |
| Fatigue | | | | | | | | | |
| EQ-5D-Y | X | | X | | | X | | X | |
| MGFA-PISⁱ | | | | | | X | | X | X |
| MGFA-Therapy Status | | | | | | X | | | |
| AChR Ab | | | | | | | | | |
| Clinical Laboratory | | | | | X | | X | X | |
| Tests | | | | | | | | | |
| Serum Pregnancy Test^{m} | | | | | | X | | X | |
| Urine Pregnancy Testⁿ | X | | X | | X | | | X | X |
| PK, Hemolysis, Free | | | | | | T/P | T | T | |
| C5^{o} | | | | | | | | | |
| ADA^{o} | | | | | | X | X | X | |
| Medically Indicated | | | | | | | X | | |
| Tests^{p} | | | | | | | | | |
| N meningitidis Vaccination^{q}* | | | | | | | | | |
| H influenzae Vaccination^{r} | | | | | | | | | |
| S pneumonia Vaccination^{r} | | | | | | | | | |
| Check for | X | X | X | X | X | X | X | X | X |
| Revaccination Status^{s} | | | | | | | | | |
| Patient Safety | X | X | X | X | X | X | X | X | X |
| Information Card^{t} | | | | | | | | | |
| Study Drug | 300 | 300 | 300 | 300 | 300 | 300 | | | |
| Infusion 10 | mg | mg | mg | mg | mg | mg | | | |
| to <20 kg^{f,u,v,w} | | | | | | | | | |
| Study Drug | 600 | 600 | 600 | 600 | 600 | 600 | | | |
| Infusion 20 | mg | mg | mg | mg | mg | mg | | | |
| to <30 kg^{f,u,v,w} | | | | | | | | | |
| Study Drug | 900 | 900 | 900 | 900 | 900 | 900 | | | |
| Infusion 30 | mg | mg | mg | mg | mg | mg | | | |
| to <40 kg^{f,u,v,w} | | | | | | | | | |
| Study Drug | 1200 | 1200 | 1200 | 1200 | 1200 | 1200 | | | |
| Infusion ≥40 Kg^{f,u,v,w} | mg | mg | mg | mg | mg | mg | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Unscheduled visits and procedures will be performed at the Investigator's discretion and results will be recorded in the eCRF.
^{c}Perform an evaluation visit for CD as soon as possible within 48 hours of notification of symptom onset. The PI may schedule additional evaluation visits at their discretion.
^{d}Patients who complete the study and transition to uninterrupted treatment with commercially available eculizumab will not be required to complete a follow-up visit.
^{e}Collect weight with minimal clothing.
^{f}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{g}Collect height with no shoes or footwear.
^{h}Serious adverse events are recorded from time of signed consent with nonserious adverse events recorded from the time of first dose.
ⁱPrior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{j}The recall period for the MG-ADL is the preceding 7 days, or since the last visit if the visit interval is <7 days
^{k}Recommendation to perform QMG assessment first to confirm eligibility. MG-ADL and MGC assessments only necessary once QMG eligibility is confirmed.
^{l}Withhold acetylcholinesterase inhibitors for at least 10 hours prior to the QMG and MGC tests.
^{m}Perform serum pregnancy tests on all female patients of childbearing potential at the specified time points. Additional pregnancy tests (serum or urine) may also be performed at any visit at the PI's discretion.
ⁿPerform urine pregnancy tests on all female patients of childbearing potential at the specified time points. Additional pregnancy tests (serum or urine) may also be performed at any visit at the PI's discretion.
^{o}Obtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion. Obtain 24-hour post-dose blood samples for PK, hemolysis, and free C5 after study drug infusion at Visit 1 (Day 1).
^{p}Any test(s) medically indicated may be performed at the discretion of the Investigator.
^{q}Patients must be vaccinated against N meningitidis if not already vaccinated within the time period of active coverage specified by the vaccine manufacturer, or vaccinate according to current medical/country guidelines at least 2 weeks prior to receiving the first dose of study drug. Patients who require vaccination against N meningitidis will be vaccinated according to current medical/country guidelines and must receive appropriate prophylactic antibiotics until 2 weeks after the vaccination if the vaccine is given less than 2 weeks prior to the first dose of study drug. Patients must remain within the vaccine manufacturer's specified period of active coverage during study participation. Patients who cannot be vaccinated must receive antibiotic prophylaxis for the entire treatment period and for 5 months following the last dose of eculizumab. Any additional vaccinations may also be performed in accordance with local guidelines.
^{r}Vaccinate patients against H influenzae and S pneumoniae, if not already vaccinated, prior to receiving the first eculizumab infusion.
^{s}Patients must be revaccinated for N meningitidis, H influenza, and S pneumoniae to provide active coverage as specified by the vaccine manufacturer or according to current medical/country guidelines.
^{t}Review the Patient Safety Information Card with the patient and caregiver to discuss the importance of carrying the safety card at all times.
^{u}Administer study drug after completion of all other tests and procedures, excluding the peak blood sampling for PK, hemolysis, and free C5 assay.
^{v}For supplemental weight-based eculizumab dosing, refer to Table 19 and Table 20.
^{w}Should the patient's weight increase ≥20 kg during the study, dosing should be based on the most recently recorded body weight at a prior dosing visit and adjusted accordingly to ensure the proper dosing regimen per patients' current weight. Refer to Table 18 for additional details on weight-based treatment regimens.
Abbreviations:
AChR Ab = acetylcholine receptor antibody;
ADA = anti-drug antibodies;
B = Baseline sample;
C5 = complement protein 5;
CD = clinical deterioration;
D = day;
ECG = electrocardiogram;
eCRF = electronic case report form;
EOS = End of Study;
EQ-5D-Y = European Quality of Life 5-Dimension Youth;
ET = Early Termination;
F/U = Follow-up;
MG = myasthenia gravis;
MG-ADL = Myasthenia Gravis Activities of Daily Living profile;
MGC = Myasthenia Gravis Composite score;
MGFA = Myasthenia Gravis Foundation of America;
MGFA-PIS = Myasthenia Gravis Foundation of America Post-Intervention Status;
N/A = not applicable;
Neuro-QoL = Quality of Life in Neurological Disorders;
P = peak sample;
PK = pharmacokinetics;
SCR = Screening;
QMG = Quantitative Myasthenia Gravis score for disease severity;
T = trough sample;
W = week.

TABLE 9
SCHEDULE OF ASSESSMENTS PART II: WEIGHT COHORT 10 TO <20 KG

| | Period/Phase | | | | | | | | | | | | | | | |
|---|---|---|---|---|---|---|---|---|---|---|---|---|---|---|---|---|
| | Extension Period (Year 1) | | | | | | | | | | | | | | | |
| | Visit Location^{a} | | | | | | | | | | | | | | | |
| | In Clinic/Remote | | | | | | In Clinic | In Clinic/Remote | | | | | In Clinic | In Clinic | | |
| | Study Visit^{b} | | | | | | | | | | | | | | | |
| | 17 | 18 | 19 | 20 | 21 | 22 | 23 | 24 | 25 | 26 | 27 | 28 | 29 | CD^{c} | ET/EOS | F/U^{d} |
| Study Weeks | W 29 | W 31 | W 33 | W 35 | W 37 | W 39 | W 41 | W 43 | W 45 | W 47 | W 49 | W 51 | W 53 | | | +W 8 |
| Window (Days) | ±2 | | | | | | | | | | | | | | | ±2 |
| Weight^{e,f} | X | X | X | X | X | X | X | X | X | X | X | X | X | X | X | X |
| Height^{g} | | | | | | | X | | | | | | X | | X | |
| Vital Signs | X | X | X | X | X | X | X | X | X | X | X | X | X | X | X | X |
| Physical Exam | | | | | | | | | | | | | X | | X | |
| 12-Lead ECG | | | | | | | | | | | | | | | X | |
| Concomitant | X | X | X | X | X | X | X | X | X | X | X | X | X | X | X | X |
| Medication | | | | | | | | | | | | | | | | |
| Adverse Event^{h} | X | X | X | X | X | X | X | X | X | X | X | X | X | X | X | X |
| MG-ADL^{i,j} | | | | | | | X | | | | | | X | X | X | X |
| QMG^{i,k} | | | | | | | X | | | | | | X | X | X | X |
| MGC^{i,k} | | | | | | | X | | | | | | X | X | X | X |
| Neuro-QoL Pediatric | | | | | | | X | | | | | | X | | X | |
| Fatigue | | | | | | | | | | | | | | | | |
| EQ-5D-Y | | | | | | | X | | | | | | X | | X | |
| MGFA-PISⁱ | | | | | | | X | | | | | | X | | X | X |
| Clinical Laboratory | | | | | | | X | | | | | | X | X | X | |
| Tests | | | | | | | | | | | | | | | | |
| Serum Pregnancy Test^{l} | | | | | | | | | | | | | | | X | |
| Urine Pregnancy Test^{m} | | X | | X | | X | | X | | X | | X | | | X | X |
| PK, Hemolysis, Free | | | | | | | | | | | | | | T | T | |
| C5ⁿ | | | | | | | | | | | | | | | | |
| ADAⁿ | | | | | | | | | | | | | | X | X | |
| Medically Indicated | | | | | | | | | | | | | | X | | |
| Tests^{o} | | | | | | | | | | | | | | | | |
| Check for | X | X | X | X | X | X | X | X | X | X | X | X | X | X | X | X |
| Revaccination Status^{p} | | | | | | | | | | | | | | | | |
| Patient Safety | X | X | X | X | X | X | X | X | X | X | X | X | X | X | X | X |
| Information Card^{q} | | | | | | | | | | | | | | | | |
| Study Drug | 300 | 300 | 300 | 300 | 300 | 300 | 300 | 300 | 300 | 300 | 300 | 300 | 300 | | | |
| Infusion 10 | mg | mg | mg | mg | mg | mg | mg | mg | mg | mg | mg | mg | mg | | | |
| to <20 kg^{f,r,s,t} | | | | | | | | | | | | | | | | |
| Study Drug | 600 | 600 | 600 | 600 | 600 | 600 | 600 | 600 | 600 | 600 | 600 | 600 | 600 | | | |
| Infusion 20 | mg | mg | mg | mg | mg | mg | mg | mg | mg | mg | mg | mg | mg | | | |
| to <30 kg^{f,r,s,t} | | | | | | | | | | | | | | | | |
| Study Drug | 900 | 900 | 900 | 900 | 900 | 900 | 900 | 900 | 900 | 900 | 900 | 900 | 900 | | | |
| Infusion 30 | mg | mg | mg | mg | mg | mg | mg | mg | mg | mg | mg | mg | mg | | | |
| to <40 kg^{f,r,s,t} | | | | | | | | | | | | | | | | |
| Study Drug | 1200 | 1200 | 1200 | 1200 | 1200 | 1200 | 1200 | 1200 | 1200 | 1200 | 1200 | 1200 | 1200 | | | |
| Infusion ≥40 Kg^{f,r,s,t} | mg | mg | mg | mg | mg | mg | mg | mg | mg | mg | mg | mg | mg | | | |
| Transition follow-up | | | | | | | | | | | | | | | X | |
| call^{u} | | | | | | | | | | | | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Unscheduled visits and procedures will be performed at the Investigator's discretion, and results will be recorded in the eCRF.
^{c}Perform an evaluation visit for CD as soon as possible within 48 hours of notification of symptom onset. The PI may schedule additional evaluation visits at their discretion.
^{d}Patients who complete the study and transition to uninterrupted treatment with commercially available eculizumab will not be required to complete a follow-up visit.
^{e}Collect weight with minimal clothing.
^{f}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{g}Collect height with no shoes or footwear.
^{h}Serious adverse events are recorded from time of signed consent, with nonserious adverse events recorded from the time of first dose.
ⁱPrior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{j}The recall period for the MG-ADL is the preceding 7 days, or since the last visit if the visit interval is <7 days.
^{k}Withhold acetylcholinesterase inhibitors for at least 10 hours prior to the QMG and MGC tests.
^{l}Perform serum pregnancy tests on all female patients of childbearing potential at the specified time points. Additional pregnancy tests (serum or urine) may also be performed at any visit at the PI's discretion.
^{m}Perform urine pregnancy tests on all female patients of childbearing potential at the specified time points. Additional pregnancy tests (serum or urine) may also be performed at any visit at the PI's discretion.
ⁿObtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion.
^{o}Any test(s) medically indicated may be performed at the discretion of the Investigator.
^{p}Patients must be revaccinated for N meningitidis, H influenza, and S pneumoniae to provide active coverage as specified by the vaccine manufacturer or according to current medical/country guidelines.
^{q}Review the Patient Safety Information Card with the patient and caregiver to discuss the importance of carrying the safety card at all times.
^{r}Administer study drug after completion of all other tests and procedures, excluding the peak blood sampling for PK, hemolysis, and free C5 assay.
^{s}For supplemental weight-based eculizumab dosing, refer to Table 19 and Table 20.
^{t}Should the patient's weight increase ≥20 kg during the study, dosing should be based on the most recently recorded body weight at a prior dosing visit and adjusted accordingly to ensure the proper dosing regimen per patient's current weight. Refer to Table 18 for additional details on weight-based treatment regimens.
^{u}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
AChR Ab = acetylcholine receptor antibody;
ADA = anti-drug antibodies;
C5 = complement protein 5;
CD = clinical deterioration;
ECG = electrocardiogram;
eCRF = electronic case report form;
EOS = End of Study;
EQ-5D-Y = European Quality of Life 5-Dimension Youth;
ET = Early Termination;
F/U = Follow-up;
MG = myasthenia gravis;
MG-ADL = Myasthenia Gravis Activities of Daily Living profile;
MGC = Myasthenia Gravis Composite score;
MGFA = Myasthenia Gravis Foundation of America;
MGFA-PIS = Myasthenia Gravis Foundation of America Post-Intervention Status;
Neuro-QoL = Quality of Life in Neurological Disorders;
PK = pharmacokinetics;
SCR = Screening;
QMG = Quantitative Myasthenia Gravis score for disease severity;
T = trough sample;
W = week.

TABLE 10
SCHEDULE OF ASSESSMENTS (EXTENSION PERIOD):
YEAR 2 THROUGH YEAR 5 (ALL WEIGHT COHORTS)

| | Phase | | | | | |
|---|---|---|---|---|---|---|
| | Extension Period | | | | | |
| | Visit Location^{a} | | | | | |
| | In Clinic/Remote | | | | | In Clinic |
| Year 2 Visit/Week^{b,c} | V31/W 54 | V32/W 56 | V33/W 58 | V34/W 60 | V35/W 62 | V36/W 64 |
| | (V30/W 55) | (V31/W 57) | (V32/W 59) | (V33/W 61) | (V34/W 63) | (V35/W 65) |
| Year 3 Visit/Week^{b,c} | V57/W 106 | V58/W 108 | V59/W 110 | V60/W 112 | V61/W 114 | V62/W 116 |
| | (V56/W 107) | (V57/W 109) | (V58/W 111) | (V59/W 113) | (V60/W 115) | (V61/W 117) |
| Year 4 Visit/Week^{b,c} | V83/W 158 | V84/W 160 | V85/W 162 | V86/W 164 | V87/W 166 | V88/W 168 |
| | (V82/W 159) | (V83/W 161) | (V84/W 163) | (V85/W 165) | (V86/W 167) | (V87/W 169) |
| Year 5 Visit/Week^{b,c} | V109/W 210 | V110/W 212 | V111/W 214 | V112/W 216 | V113/W 218 | V114/W 220 |
| | (V108/W 211) | (V109/W 213) | (V110/W 215) | (V111/W 217) | (V112/W 219) | (V113/W 221) |
| Window (Days) | ±2 | | | | | |
| Weight^{f,g} | X | X | X | X | X | X |
| Height^{h} | | | | | | X |
| Vital Signs | X | X | X | X | X | X |
| Physical Exam | | | | | | |
| 12-Lead ECG | | | | | | |
| Concomitant | X | X | X | X | X | X |
| Medication | | | | | | |
| Adverse Eventⁱ | X | X | X | X | X | X |
| MG-ADL^{j,k} | | | | | | X |
| QMG^{j,l} | | | | | | X |
| MGC^{j,l} | | | | | | X |
| Neuro-QoL Pediatric | | | | | | X |
| Fatigue | | | | | | |
| EQ-5D-Y | | | | | | X |
| MGFA-PIS^{j} | | | | | | |
| Clinical Laboratory | | | | | | |
| Tests^{p} | | | | | | |
| Serum Pregnancy Test^{m} | | | | | | |
| Urine Pregnancy Testⁿ | | | | | | |
| PK, Hemolysis, Free | | | | | | X |
| C5^{o} | | | | | | |
| ADA^{o} | | | | | | X |
| Medically Indicated | | | | | | |
| Tests | | | | | | |
| Check for | X | X | X | X | X | X |
| Revaccination Status^{q} | | | | | | |
| Patient Safety | X | X | X | X | X | X |
| Information Card^{r} | | | | | | |
| Study Drug | 300 | 300 | 300 | 300 | 300 | 300 |
| Infusion 10 | mg | mg | mg | mg | mg | mg |
| to <20 kg^{g,s,t,u} | | | | | | |
| Study Drug | 600 | 600 | 600 | 600 | 600 | 600 |
| Infusion 20 | mg | mg | mg | mg | mg | mg |
| to <30 kg^{g,s,t,u} | | | | | | |
| Study Drug | 900 | 900 | 900 | 900 | 900 | 900 |
| Infusion 30 | mg | mg | mg | mg | mg | mg |
| to <40 kg^{g,s,t,u} | | | | | | |
| Study Drug | 1200 | 1200 | 1200 | 1200 | 1200 | 1200 |
| Infusion >40 kg^{g,s,t,u} | mg | mg | mg | mg | mg | mg |
| Transition follow-up | | | | | | |
| call^{v} | | | | | | |

| | Phase | | | | |
|---|---|---|---|---|---|
| | Extension Period | | | | |
| | Visit Location^{a} | | | | |
| | In Clinic/Remote | | In Clinic | | |
| Year 2 Visit/Week^{b,c} | V37/W 66 | V38/W 68 | CD^{d} | ET/EOS | F/U^{e} |
| | (V36/W 67) | (V37/W 69) | | | (+W 8) |
| Year 3 Visit/Week^{b,c} | V63/W 118 | V64/W 120 | CD^{d} | ET/EOS | F/U^{e} |
| | (V62/W 119) | (V63/W 121) | | | (+W 8) |
| Year 4 Visit/Week^{b,c} | V89/W 170 | V90/W 172 | CD^{d} | ET/EOS | F/U^{e} |
| | (V88/W 171) | (V89/W 173) | | | (+W 8) |
| Year 5 Visit/Week^{b,c} | V115/W 222 | V116/W 224 | CD^{d} | ET/EOS | F/U^{e} |
| | (V114/W 223) | (V115/W 225) | | | (+W 8) |
| Window (Days) | ±2 | | | | (±2) |
| Weight^{f,g} | X | X | X | X | X |
| Height^{h} | | | | X | |
| Vital Signs | X | X | X | X | X |
| Physical Exam | | | | X | |
| 12-Lead ECG | | | | X | |
| Concomitant | X | X | X | X | X |
| Medication | | | | | |
| Adverse Eventⁱ | X | X | X | X | X |
| MG-ADL^{j,k} | | | X | X | X |
| QMG^{j,l} | | | X | X | X |
| MGC^{j,l} | | | X | X | X |
| Neuro-QoL Pediatric | | | | X | |
| Fatigue | | | | | |
| EQ-5D-Y | | | | X | |
| MGFA-PIS^{j} | | | | X | X |
| Clinical Laboratory | | | X | X | |
| Tests^{p} | | | | | |
| Serum Pregnancy Test^{m} | | | | X | |
| Urine Pregnancy Testⁿ | | | | X | X |
| PK, Hemolysis, Free | | | T | T | |
| C5^{o} | | | | | |
| ADA^{o} | | | X | X | |
| Medically Indicated | | | X | | |
| Tests | | | | | |
| Check for | X | X | X | X | X |
| Revaccination Status^{q} | | | | | |
| Patient Safety | X | X | X | X | X |
| Information Card^{r} | | | | | |
| Study Drug | 300 | 300 | | | |
| Infusion 10 | mg | mg | | | |
| to <20 kg^{g,s,t,u} | | | | | |
| Study Drug | 600 | 600 | | | |
| Infusion 20 | mg | mg | | | |
| to <30 kg^{g,s,t,u} | | | | | |
| Study Drug | 900 | 900 | | | |
| Infusion 30 | mg | mg | | | |
| to <40 kg^{g,s,t,u} | | | | | |
| Study Drug | 1200 | 1200 | | | |
| Infusion >40 kg^{g,s,t,u} | mg | mg | | | |
| Transition follow-up | | | | X | |
| call^{v} | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Shown as visit/week for Weight Cohorts ≥40 kg, 30 to <40 kg, and 20 to <30 kg (10 to <20 kg).
^{c}Unscheduled visits and procedures will be performed at the Investigator's discretion, and results will be recorded in the eCRF.
^{d}Perform an evaluation visit for CD as soon as possible within 48 hours of notification of symptom onset. The PI may schedule additional evaluation visits at their discretion.
^{e}Patients who complete the study and transition to uninterrupted treatment with commercially available eculizumab will not be required to complete a follow-up visit.
^{f}Collect weight with minimal clothing.
^{g}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{h}Collect height with no shoes or footwear. Serious adverse events are recorded from time of signed consent, with nonserious adverse events recorded from the time of first dose.
ⁱPrior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{j}The recall period for the MG-ADL is the preceding 7 days, or since the last visit if the visit interval is <7 days.
^{k}Withhold acetylcholinesterase inhibitors for at least 10 hours prior to the QMG and MGC tests.
^{l}Perform serum pregnancy tests on all female patients of childbearing potential at the specified time points. Additional pregnancy tests (serum or urine) may also be performed at any visit at the PI's discretion.
^{m}Perform urine pregnancy tests on all female patients of childbearing potential at the specified time points. Additional pregnancy tests (serum or urine) may also be performed at any visit at the PI's discretion.
ⁿObtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion.
^{o}Any test(s) medically indicated may be performed at the discretion of the Investigator.
^{p}Patients must be revaccinated for N meningitidis, H influenza, and S pneumoniae to provide active coverage as specified by the vaccine manufacturer or according to current medical/country guidelines.
^{q}Review the Patient Safety Information Card with the patient and caregiver to discuss the importance of carrying the safety card at all times.
^{r}Administer study drug after completion of all other tests and procedures, excluding the peak blood sampling for PK, hemolysis, and free C5 assay.
^{s}For supplemental weight-based eculizumab dosing, refer to Table 19 and Table 20.
^{t}Should the patient's weight increase ≥20 kg during the study, dosing should be based on the most recently recorded body weight at a prior dosing visit and adjusted accordingly to ensure the proper dosing regimen per patients' current weight. Refer to Table 18 for additional details on weight-based treatment regimens.
^{u}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
ADA = antidrug antibodies;
C5 = complement protein 5;
CD = clinical deterioration;
ECG = electrocardiogram;
eCRF = electronic case report form;
EOS = End of Study;
EQ-5D-Y = European Quality of life 5 Dimension Youth;
ET = early termination;
MG = myasthenia gravis;
MG-ADL = Myasthenia Gravis Activities of Daily Living profile;
MGC = Myasthenia Gravis Composite;
MGFA-PIS = Myasthenia Gravis Foundation of America Post-Interventional Status;
Neuro-QoL = Quality of Life in Neurological Disorders;
PI = Principal Investigator;
PK = pharmacokinetics;
QMG = Quantitative Myasthenia Gravis Score for Disease Severity;
T = trough sample;
V = visit;
W = week.

TABLE 11
SCHEDULE OF ASSESSMENTS (EXTENSION PERIOD): YEAR
2 THROUGH YEAR 5 (ALL WEIGHT COHORTS), CONTINUED

| | Phase | | | | | |
|---|---|---|---|---|---|---|
| | Extension Period | | | | | |
| | Visit Location^{a} | | | | | |
| | In Clinic/Remote | | | | | In Clinic |
| Year 2 Visit/Week^{b,c} | V39/W 70 | V40/W 72 | V41/W 74 | V42/W 76 | V43/W 78 | V44/W 80 |
| | (V38/W 71) | (V39/W 73) | (V40/W 75) | (V41/W 77) | (V42/W 79) | (V43/W 81) |
| Year 3 Visit/Week^{b,c} | V65/W 122 | V66/W 124 | V67/W 126 | V68/W 128 | V69/W 130 | V70/W 132 |
| | (V64/W 123) | (V65/W 125) | (V66/W 127) | (V67/W 129) | (V68/W 131) | (V69/W 133) |
| Year 4 Visit/Week^{b,c} | V91/W 174 | V92/W 176 | V93/W 178 | V94/W 180 | V95/W 182 | V96/W 184 |
| | (V90/W 175) | (V91/W 177) | (V92/W 179) | (V93/W 181) | (V94/W 183) | (V95/W 185) |
| Year 5 Visit/Week^{b,c} | V117/W 226 | V118/W 228 | V119/W 230 | V120/W 232 | (Refer to | NA |
| | (V116/W 227) | (V117/W 229) | (V118/W 231) | (V119/W 233) | EOS) | |
| Window (Days) | ±2 | | | | | |
| Weight^{f,g} | X | X | X | X | X | X |
| Height^{h} | | | | | X | |
| Vital Signs | X | X | X | X | X | X |
| Physical Exam | | | | | | |
| 12-Lead ECG | | | | | | |
| Concomitant | X | X | X | X | X | X |
| Medication | | | | | | |
| Adverse Eventⁱ | X | X | X | X | X | X |
| MG-ADL^{j,k} | | | | | X | |
| QMG^{j,l} | | | | | X | |
| MGC^{j,l} | | | | | X | |
| Neuro-QoL Pediatric | | | | | X | |
| Fatigue | | | | | | |
| EQ-5D-Y | | | | | X | |
| MGFA-PIS^{j} | | | | | | |
| Clinical Laboratory | | | | | | |
| Tests | | | | | | |
| Serum Pregnancy Test^{m} | | | | | | |
| Urine Pregnancy Testⁿ | | | | | | |
| PK, Hemolysis, Free | | | | | X | |
| C5^{o} | | | | | | |
| ADA^{o} | | | | | X | |
| Medically Indicated | | | | | | |
| Tests^{p} | | | | | | |
| Check for | X | X | X | X | X | X |
| Revaccination Status^{q} | | | | | | |
| Patient Safety | X | X | X | X | X | X |
| Information Card^{r} | | | | | | |
| Study Drug | 300 | 300 | 300 | 300 | 300 | 300 |
| Infusion 10 | mg | mg | mg | mg | mg | mg |
| to <20 kg^{g,s,t,u} | | | | | | |
| Study Drug | 600 | 600 | 600 | 600 | 600 | 600 |
| Infusion 20 | mg | mg | mg | mg | mg | mg |
| to <30 kg^{g,s,t,u} | | | | | | |
| Study Drug | 900 | 900 | 900 | 900 | 900 | 900 |
| Infusion 30 | mg | mg | mg | mg | mg | mg |
| to <40 kg^{g,s,t,u} | | | | | | |
| Study Drug | 1200 | 1200 | 1200 | 1200 | 1200 | 1200 |
| Infusion 240 kg^{g,s,t,u} | mg | mg | mg | mg | mg | mg |
| Transition follow-up | | | | | | |
| call^{v} | | | | | | |

| | Phase | | | | |
|---|---|---|---|---|---|
| | Extension Period | | | | |
| | Visit Location^{a} | | | | |
| | In Clinic/Remote | | In Clinic | | |
| Year 2 Visit/Week^{b,c} | V45/W 82 | V46/W 84 | CD^{d} | ET/EOS | F/U^{e} |
| | (V44/W 83) | (V45/W 85) | | | (+W 8) |
| Year 3 Visit/Week^{b,c} | V71/W 134 | V72/W 136 | CD^{d} | ET/EOS | F/U^{e} |
| | (V70/W 135) | (V71/W 137) | | | (+W 8) |
| Year 4 Visit/Week^{b,c} | V97/W 186 | V98/W 188 | CD^{d} | ET /EOS | F/U^{e} |
| | (V96/W 187) | (V97/W 189) | | | (+W 8) |
| Year 5 Visit/Week^{b,c} | NA | NA | CD^{d} | ET/EOS/ | F/U^{e} |
| | | | | V121/W 234 | (+W 8) |
| | | | | (V120/W 235) | |
| Window (Days) | ±2 | | | | (±2) |
| Weight^{f,g} | X | X | X | X | X |
| Height^{h} | | | | X | |
| Vital Signs | X | X | X | X | X |
| Physical Exam | | | | X | |
| 12-Lead ECG | | | | X | |
| Concomitant | X | X | X | X | X |
| Medication | | | | | |
| Adverse Eventⁱ | X | X | X | X | X |
| MG-ADL^{j,k} | | | X | X | X |
| QMG^{j,l} | | | X | X | X |
| MGC^{j,l} | | | X | X | X |
| Neuro-QoL Pediatric | | | | X | |
| Fatigue | | | | | |
| EQ-5D-Y | | | | X | |
| MGFA-PIS^{j} | | | | X | X |
| Clinical Laboratory | | | X | X | |
| Tests | | | | | |
| Serum Pregnancy Test^{m} | | | | X | |
| Urine Pregnancy Testⁿ | | | | X | X |
| PK, Hemolysis, Free | | | T | T | |
| C5^{o} | | | | | |
| ADA^{o} | | | X | X | |
| Medically Indicated | | | X | | |
| Tests^{p} | | | | | |
| Check for | X | X | X | X | X |
| Revaccination Status^{q} | | | | | |
| Patient Safety | X | X | X | X | X |
| Information Card^{r} | | | | | |
| Study Drug | 300 | 300 | | | |
| Infusion 10 | mg | mg | | | |
| to <20 kg^{g,s,t,u} | | | | | |
| Study Drug | 600 | 600 | | | |
| Infusion 20 | mg | mg | | | |
| to <30 kg^{g,s,t,u} | | | | | |
| Study Drug | 900 | 900 | | | |
| Infusion 30 | mg | mg | | | |
| to <40 kg^{g,s,t,u} | | | | | |
| Study Drug | 1200 | 1200 | | | |
| Infusion 240 kg^{g,s,t,u} | mg | mg | | | |
| Transition follow-up | | | | X | |
| call^{v} | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Shown as visit/week for Weight Cohorts 240 kg, 30 to <40 kg, and 20 to <30 kg (10 to <20 kg).
^{c}Unscheduled visits and procedures will be performed at the Investigator's discretion, and results will be recorded in the eCRF.
^{d}Perform an evaluation visit for CD as soon as possible within 48 hours of notification of symptom onset. The PI may schedule additional evaluation visits at their discretion.
^{e}Patients who complete the study and transition to uninterrupted treatment with commercially available eculizumab will not be required to complete a follow-up visit.
^{f}Collect weight with minimal clothing.
^{g}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{h}Collect height with no shoes or footwear.
ⁱSerious adverse events are recorded from time of signed consent, with nonserious adverse events recorded from the time of first dose.
^{j}Prior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{l}The recall period for the MG-ADL is the preceding 7 days, or since the last visit if the visit interval is <7 days. Withhold acetylcholinesterase inhibitors for at least 10 hours prior to the QMG and MGC tests.
^{m}Perform serum pregnancy tests on all female patients of childbearing potential at the specified time points. Additional pregnancy tests (serum or urine) may also be performed at any visit at the PI's discretion.
ⁿPerform urine pregnancy tests on all female patients of childbearing potential at the specified time points. Additional pregnancy tests (serum or urine) may also be performed at any visit at the PI's discretion.
^{o}Obtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion.
^{p}Any test(s) medically indicated may be performed at the discretion of the Investigator.
^{q}Patients must be revaccinated for N meningitidis, H influenza, and S pneumoniae to provide active coverage as specified by the vaccine manufacturer or according to current medical/country guidelines.
^{r}Review the Patient Safety information Card with the patient and caregiver to discuss the importance of carrying the safety card at all times.
^{s}Administer study drug after completion of all other tests and procedures, excluding the peak blood sampling for PK, hemolysis, and free C5 assay.
^{t}For supplemental weight-based eculizumab dosing, refer to Table 19 and Table 20.
^{u}Should the patient's weight increase ≥20 kg during the study, dosing should be based on the most recently recorded body weight at a prior dosing visit and adjusted accordingly to ensure the proper dosing regimen per patients' current weight. Refer to Table 18 for additional details on weight-based treatment regimens.
^{v}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
ADA = antidrug antibodies;
C5 = complement protein 5;
CD = clinical deterioration;
ECG = electrocardiogram;
eCRF = electronic case report form;
EQ-5D-Y = European Quality of life 5 Dimension Youth;
ET = early termination;
F/U = Follow-up;
MG = myasthenia gravis;
MG-ADL = Myasthenia Gravis Activities of Daily Living profile;
MGC = Myasthenia Gravis Composite;
MGFA-PIS = Myasthenia Gravis Foundation of America Post-Interventional Status;
NA = not applicable;
Neuro-QoL = Quality of Life in Neurological Disorders;
PI = Principal Investigator;
PK = pharmacokinetics;
QMG = Quantitative Myasthenia Gravis Score for Disease Severity;
T = trough sample;
V = visit;
W = week.

TABLE 12
SCHEDULE OF ASSESSMENTS (EXTENSION PERIOD): YEAR
2 THROUGH YEAR 5 (ALL WEIGHT COHORTS), CONTINUED

| | Phase | | | | |
|---|---|---|---|---|---|
| | Extension Period | | | | |
| | Visit Location^{a} | | | | |
| | In Clinic/Remote | In Clinic | In Clinic/Remote | | |
| Year 2 Visit/Week^{b,c} | V47/W 86 | V48/W 88 | V49/W 90 | V50/W 92 | V51/W 94 |
| | (V46/W 87) | (V47/W 89) | (V48/W 91) | (V49/W 93) | (V50/W 95) |
| Year 3 Visit/Week^{b,c} | V73/W 138 | V74/W 140 | V75/W 142 | V76/W 144 | V77/W 146 |
| | (V72/W 139) | (V73/W 141) | (V74/W 143) | (V75/W 145) | (V76/W 147) |
| Year 4 Visit/Week^{b,c} | V99/W 190 | V100/W 192 | V101/W 194 | V102/W 196 | V103/W 198 |
| | (V98/W 191) | (V99/W 193) | (V100/W 195) | (V101/W 197) | (V102/W 199) |
| Year 5 Visit/Week^{b,c} | NA | NA | NA | NA | NA |
| Window (Days) | ±2 | | | | |
| Weight^{f,g} | X | X | X | X | X |
| Height^{h} | | | X | | |
| Vital Signs | X | X | X | X | X |
| Physical Exam | | | | | |
| 12-Lead ECG | | | | | |
| Concomitant | X | X | X | X | X |
| Medication | | | | | |
| Adverse Eventⁱ | X | X | X | X | X |
| MG-ADL^{j,k} | | | X | | |
| QMG^{j,l} | | | X | | |
| MGC^{j,l} | | | X | | |
| Neuro-QoL Pediatric | | | X | | |
| Fatigue | | | | | |
| EQ-5D-Y | | | X | | |
| MGFA-PIS^{j} | | | | | |
| Clinical Laboratory | | | | | |
| Tests | | | | | |
| Serum Pregnancy Test^{m} | | | | | |
| Urine Pregnancy Testⁿ | | | | | |
| PK, Hemolysis, Free | | | X | | |
| C5^{o} | | | | | |
| ADA^{o} | | | X | | |
| Medically Indicated | | | | | |
| Tests^{p} | | | | | |
| Check for | X | X | X | X | X |
| Revaccination Status^{q} | | | | | |
| Patient Safety | X | X | X | X | X |
| Information Card^{r} | | | | | |
| Study Drug | 300 | 300 | 300 | 300 | 300 |
| Infusion 10 | mg | mg | mg | mg | mg |
| to <20 kg^{g,s,t,u} | | | | | |
| Study Drug | 600 | 600 | 600 | 600 | 600 |
| Infusion 20 | mg | mg | mg | mg | mg |
| to <30 kg^{g,s,t,u} | | | | | |
| Study Drug | 900 | 900 | 900 | 900 | 900 |
| Infusion 30 | mg | mg | mg | mg | mg |
| to <40 kg^{g,s,t,u} | | | | | |
| Study Drug | 1200 | 1200 | 1200 | 1200 | 1200 |
| Infusion ≥40 kg^{g,s,t,u} | mg | mg | mg | mg | mg |
| Transition follow-up | | | | | |
| call^{v} | | | | | |

| | Phase | | | | | |
|---|---|---|---|---|---|---|
| | Extension Period | | | | | |
| | Visit Location^{a} | | | | | |
| | In Clinic/Remote | | In Clinic | | | |
| Year 2 Visit/Week^{b,c} | V52/W 96 | V53/W 98 | V54/W 100 | CD^{d} | ET/EOS | F/U^{e} |
| | (V51/W 97) | (V52/W 99) | (V53/W 101) | | | (+W 8) |
| Year 3 Visit/Week^{b,c} | V78/W 148 | V79/W 150 | V80/W 152 | CD^{d} | ET/EOS | F/U^{e} |
| | (V77/W 149) | (V78/W 151) | (V79/W 153) | | | (+W 8) |
| Year 4 Visit/Week^{b,c} | V104/W 200 | V105/W 202 | V106/W 204 | CD^{d} | ET/EOS | F/U^{e} |
| | (V103/W 201) | (V104/W 203) | (V105/W 205) | | | (+W 8) |
| Year 5 Visit/Week^{b,c} | NA | NA | NA | NA | NA | NA |
| Window (Days) | ±2 | | | | | (±2) |
| Weight^{f,g} | X | X | X | X | X | X |
| Height^{h} | | | | | X | |
| Vital Signs | X | X | X | X | X | X |
| Physical Exam | | | | | X | |
| 12-Lead ECG | | | | | X | |
| Concomitant | X | X | X | X | X | X |
| Medication | | | | | | |
| Adverse Eventⁱ | X | X | X | X | X | X |
| MG-ADL^{j,k} | | | | X | X | X |
| QMG^{j,l} | | | | X | X | X |
| MGC^{j,l} | | | | X | X | X |
| Neuro-QoL Pediatric | | | | | X | |
| Fatigue | | | | | | |
| EQ-5D-Y | | | | | X | |
| MGFA-PIS^{j} | | | | | X | X |
| Clinical Laboratory | | | | X | X | |
| Tests | | | | | | |
| Serum Pregnancy Test^{m} | | | | | X | |
| Urine Pregnancy Testⁿ | | | | | X | X |
| PK, Hemolysis, Free | | | | T | T | |
| C5^{o} | | | | | | |
| ADA^{o} | | | | X | X | |
| Medically Indicated | | | | X | | |
| Tests^{p} | | | | | | |
| Check for | X | X | X | X | X | X |
| Revaccination Status^{q} | | | | | | |
| Patient Safety | X | X | X | X | X | X |
| Information Card^{r} | | | | | | |
| Study Drug | 300 | 300 | 300 | | | |
| Infusion 10 | mg | mg | mg | | | |
| to <20 kg^{g,s,t,u} | | | | | | |
| Study Drug | 600 | 600 | 600 | | | |
| Infusion 20 | mg | mg | mg | | | |
| to <30 kg^{g,s,t,u} | | | | | | |
| Study Drug | 900 | 900 | 900 | | | |
| Infusion 30 | mg | mg | mg | | | |
| to <40 kg^{g,s,t,u} | | | | | | |
| Study Drug | 1200 | 1200 | 1200 | | | |
| Infusion ≥40 kg^{g,s,t,u} | mg | mg | mg | | | |
| Transition follow-up | | | | | X | |
| call^{v} | | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Shown as visit/week for Weight Cohorts ≥40 kg, 30 to <40 kg, and 20 to <30 kg (10 to <20 kg).
^{c}Unscheduled visits and procedures will be performed at the Investigator's discretion, and results will be recorded in the eCRF.
^{d}Perform an evaluation visit for CD as soon as possible within 48 hours of notification of symptom onset. The PI may schedule additional evaluation visits at their discretion.
^{e}Patients who complete the study and transition to uninterrupted treatment with commercially available eculizumab will not be required to complete a follow-up visit.
^{f}Collect weight with minimal clothing.
^{g}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{h}Collect height with no shoes or footwear.
ⁱSerious adverse events are recorded from time of signed consent, with nonserious adverse events recorded from the time of first dose.
^{j}Prior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{k}The recall period for the MG-ADL is the preceding 7 days, or since the last visit if the visit interval is <7 days.
^{l}Withhold acetylcholinesterase inhibitors for at least 10 hours prior to the QMG and MGC tests.
^{m}Perform serum pregnancy tests on all female patients of childbearing potential at the specified time points. Additional pregnancy tests (serum or urine) may also be performed at any visit at the PI's discretion.
ⁿPerform urine pregnancy tests on all female patients of childbearing potential at the specified time points. Additional pregnancy tests (serum or urine) may also be performed at any visit at the PI's discretion.
^{o}Obtain Baseline and trough blood samples for PK, hemolysis, free C5, and ADA tests 5-90 minutes before study drug infusion. Obtain peak blood samples for PK, hemolysis, and free C5 tests at least 60 minutes after the completion of study drug infusion.
^{p}Any test(s) medically indicated may be performed at the discretion of the Investigator.
^{q}Patients must be revaccinated for N meningitidis, H influenza, and S pneumoniae to provide active coverage as specified by the vaccine manufacturer or according to current medical/country guidelines.
^{r}Review the Patient Safety Information Card with the patient and caregiver to discuss the importance of carrying the safety card at all times.
^{s}Administer study drug after completion of all other tests and procedures, excluding the peak blood sampling for PK, hemolysis, and free C5 assay.
^{t}For supplemental weight-based eculizumab dosing, refer to Table 19 and Table 20.
^{u}Should the patient's weight increase ≥20 kg during the study, dosing should be based on the most recently recorded body weight at a prior dosing visit and adjusted accordingly to ensure the proper dosing regimen per patients' current weight. Refer to Table 18 for additional details on weight-based treatment regimens.
^{v}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
ADA = antidrug antibodies;
C5 = complement protein 5;
CD = clinical deterioration;
ECG = electrocardiogram;
eCRF = electronic case report form;
EOS = End of Study;
EQ-5D-Y = European Quality of life 5 Dimension Youth;
ET = early termination;
F/U = Follow-up;
MG = myasthenia gravis;
MG-ADL = Myasthenia Gravis Activities of Daily Living profile;
MGC = Myasthenia Gravis Composite;
MGFA-PIS = Myasthenia Gravis Foundation of America Post-Interventional Status;
Neuro-QoL = Quality of Life in Neurological Disorders;
PI = Principal Investigator;
PK = pharmacokinetics;
QMG = Quantitative Myasthenia Gravis Score for Disease Severity;
T = trough sample;
V = visit;
W = week.

TABLE 13
SCHEDULE OF ASSESSMENTS (EXTENSION PERIOD): YEAR
2 THROUGH YEAR 5 (ALL WEIGHT COHORTS), CONTINUED

| | Phase | | | | |
|---|---|---|---|---|---|
| | Extension Period | | | | |
| | Visit Location^{a} | | | | |
| | | | In Clinic | | |
| Year 2 Visit/Week^{b,c} | V55/W 102 | V56/W 104 | CD^{d} | ET/EOS | F/U^{e} |
| | (V54/W 103) | (V55/W 105) | | | (+W 8) |
| Year 3 Visit/Week^{b,c} | V81/W 154 | V82/W 156 | CD^{d} | ET/EOS | F/U^{e} |
| | (V80/W 155) | (V81/W 157) | | | (+W 8) |
| Year 4 Visit/Week^{b,c} | V107/W 206 | V108/W 208 | CD^{d} | ET/EOS | F/U^{e} |
| | (V106/W 207) | (V107/W 209) | | | (+W 8) |
| Year 5 Visit/Week^{b,c} | NA | NA | NA | NA | NA |
| Window (Days) | ±2 | | | | ±2 |
| Weight^{f,g} | X | X | X | X | X |
| Height^{h} | | X | | X | |
| Vital Signs | X | X | X | X | X |
| Physical Exam | | | | X | |
| 12-Lead ECG | | | | X | |
| Concomitant | X | X | X | X | X |
| Medication | | | | | |
| Adverse Eventⁱ | X | X | X | X | X |
| MG-ADL^{j,k} | | X | X | X | X |
| QMG^{j,l} | | X | X | X | X |
| MGC^{j,l} | | X | X | X | X |
| Neuro-QoL Pediatric | | X | | X | |
| Fatigue | | | | | |
| EQ-5D-Y | | X | | X | |
| MGFA-PIS^{j} | | | | X | X |
| Clinical Laboratory | | | X | X | |
| Tests | | | | | |
| Serum Pregnancy Test^{m} | | | | X | |
| Urine Pregnancy Testⁿ | | | | X | X |
| PK, Hemolysis, Free | | X | T | T | |
| C5^{o} | | | | | |
| ADA^{o} | | X | X | X | |
| Medically Indicated | | | X | | |
| Tests^{p} | | | | | |
| Check for | X | X | X | X | X |
| Revaccination Status^{q} | | | | | |
| Patient Safety | X | X | X | X | X |
| Information Card^{r} | | | | | |
| Study Drug | 300 | 300 | | | |
| Infusion 10 | mg | mg | | | |
| to <20 kg^{g,s,t,u} | | | | | |
| Study Drug | 600 | 600 | | | |
| Infusion 20 | mg | mg | | | |
| to <30 kg^{g,s,t,u} | | | | | |
| Study Drug | 900 | 900 | | | |
| Infusion 30 | mg | mg | | | |
| to <40 kg^{g,s,t,u} | | | | | |
| Study Drug | 1200 | 1200 | | | |
| Infusion ≥40 kg^{g,s,t,u} | mg | mg | | | |
| Transition follow-up | | | | X | |
| call^{v} | | | | | |

^{a}In Clinic - visits must be conducted at the investigational sites; Remote - visits may be conducted remotely at a medical facility that is located near the subject's home or at the subject's home with the permission of the Investigator in accordance with all national, state, and local laws or regulations of the pertinent regulatory authorities.
^{b}Shown as visit/week for Weight Cohorts ≥40 kg, 30 to <40 kg, and 20 to <30 kg (10 to <20 kg).
^{c}Unscheduled visits and procedures will be performed at the Investigator's discretion, and results will be recorded in the eCRF.
^{d}Perform an evaluation visit for CD as soon as possible within 48 hours of notification of symptom onset. The PI may schedule additional evaluation visits at their discretion.
^{e}Patients who complete the study and transition to uninterrupted treatment with commercially available eculizumab will not be required to complete a follow-up visit.
^{f}Collect weight with minimal clothing.
^{g}Weight changes during the development and maturation of pediatric patients impact the dosing profile of eculizumab and should be adjusted to accommodate any growth changes in patients. Dosing should be based on the most recently recorded body weight at a prior dosing visit. For additional details on weight-based treatment regimens, refer to Table 18 for scheduled doses of eculizumab and Table 19 and Table 20 for supplemental doses of eculizumab.
^{h}Collect height with no shoes or footwear.
ⁱSerious adverse events are recorded from time of signed consent, with nonserious adverse events recorded from the time of first dose.
^{j}Prior to study drug infusion, perform the MG-ADL, QMG, and MGC using a properly trained evaluator, preferably the same evaluator at approximately the same time of day throughout the study; MGFA-PIS must be performed by the PI or suitably-trained neurologist, preferably the same evaluator throughout the study.
^{k}The recall period for the MG-ADL is the preceding 7 days, or since the last visit if the visit interval is <7 days.
^{l}Withhold acetylcholinesterase inhibitors for at least 10 hours prior to the QMG and MGC tests.
^{m}Perform serum pregnancy tests on all female patients of childbearing potential at the specified time points. Additional pregnancy tests (serum or urine) may also be performed at any visit at the PI's discretion.
ⁿPerform urine pregnancy tests on all female patients of childbearing potential at the specified time points. Additional pregnancy tests (serum or untie) may also be performed at any visit at the PI's discretion.
^{o}Obtain Baseline and trough blood samples for PK, hemolysis, free C5, and ADA tests 5-90 minutes before study drug infusion Obtain peak blood samples for PK, hemolysis, and free C5 tests at least 60 minutes after the completion of study drug infusion.
^{p}Any test(s) medically indicated may be performed at the discretion of the Investigator.
^{q}Patients must be revaccinated for N meningitidis, H influenza, and S pneumoniae to provide active coverage as specified by the vaccine manufacturer or according to current medical/country guidelines.
^{r}Review the Participant Safety Information Card with the patient and caregiver to discuss the importance of carrying the safety card at all times.
^{s}Administer study drug after completion of all other tests and procedures, excluding the peak blood sampling for PK, hemolysis, and free C5 assay.
^{t}For supplemental weight-based eculizumab dosing please refer to Table 19 and Table 20.
^{u}Should the patient's weight increase ≥20 kg during the study, dosing should be based on the most recently recorded body weight at a prior dosing visit and adjusted accordingly to ensure the proper dosing regimen per patients' current weight. Refer to Table 18 for additional details on weight-based treatment regimens.
^{v}The Investigator must confirm with the patient or their guardian/caregiver by telephone that the transition to commercially available eculizumab occurred within 2 weeks of the last scheduled dose during the study. In the event that treatment with commercially available eculizumab is delayed, an unscheduled safety Follow-up Visit should occur on the day of initiating commercial eculizumab treatment or as soon as feasible.
Abbreviations:
ADA = antidrug antibodies;
C5 = complement protein 5;
CD = clinical deterioration;
ECG = electrocardiogram;
eCRF = electronic case report form;
EOS = End of Study;
EQ-5D-Y = European Quality of life 5 Dimension Youth;
ET = early termination;
F/U = Follow-up;
MG = myasthenia gravis;
MG-ADL = Myasthenia Gravis Activities of Daily Living profile;
MGC = Myasthenia Gravis Composite;
MGFA-PIS = Myasthenia Gravis Foundation of America Post-Interventional Status;
Neuro-QoL = Quality of Life in Neurological Disorders;
PI = Principal Investigator;
PK = pharmacokinetics;
QMG = Quantitative Myasthenia Gravis Score for Disease Severity;
T = trough sample;
V = visit;
W = week.

### 1.3. Standard Protocol Definitions

Abbreviations and definitions for the study and follow-up period are provided in Table 14.

TABLE 14
LIST OF ABBREVIATIONS AND DEFINITIONS OF TERMS

| Abbreviations | |
|---|---|
| and Terms | Definitions |
| Ab, Abs | antibody, antibodies |
| AChR | acetylcholine receptor |
| AChI | acetylcholinesterase inhibitor |
| ADA | antidrug antibody |
| AE | adverse event |
| aHUS | atypical hemolytic uremic syndrome |
| AZA | azathioprine |
| C5 | complement protein 5 |
| Cmax | maximum plasma drug concentration |
| ECG | electrocardiogram |
| eCRF | electronic Case Report Form |
| EOS | end-of-study |
| EQ-5D-Y | European Quality of Life 5-Dimension Youth version |
| ET | early termination |
| EU | European Union |
| FAS | Full Analysis Set |
| FFP | fresh frozen plasma |
| FVC | forced vital capacity |
| GCP | Good Clinical Practices |
| gMG | generalized myasthenia gravis |
| GPV | Global Pharmacovigilance |
| H influenzae | Haemophilus influenzae |
| IB | Investigator's Brochure |
| ICF | informed consent form |
| ICH | International Council on Harmonization |
| IEC | Institutional (or Independent) Ethics Committee |
| IRB | Institutional Review Board |
| IST | immunosuppressant therapy |
| IV | intravenous |
| IVIg | intravenous immunoglobulin |
| IXRS | Interactive Voice or Web Response System |
| MedDRA | Medical Dictionary for Regulatory/Activities |
| mFAS | Modified Full Analysis Set |
| MG | myasthenia gravis |
| MG-ADL | Myasthenia Gravis Activities of Daily Living profile |
| MGC | Myasthenia Gravis Composite Scale |
| MGFA | Myasthenia Gravis Foundation of America |
| MMF | mycophenolate mofetil |
| MMT | manual muscle test |
| MTX | methotrexate |
| N meningitidis | Neisseria meningitidis |
| Neuro-QoL | Neurological Quality of Life-Fatigue questionnaire |
| Fatigue | |
| NMJ | neuromuscular junction |
| PD | pharmacodynamics |
| PDCO | Paediatric Committee of the European Medicines |
| PE | plasma exchange |
| PI | Principal Investigator |
| PIS | post-intervention status |
| PK | pharmacokinetics |
| PNH | paroxysmal nocturnal hemoglobinuria |
| PP | plasmapheresis |
| PT | preferred term |
| QMG | Quantitative Myasthenia Gravis |
| S pneumoniae | Streptococcus pneumoniae |
| SAE | serious adverse event |
| SAP | statistical analysis plan |
| SOC | System Organ Class |
| TEAE | treatment-emergent adverse event |
| VAS | Visual Analog Scale |
| WHODrug | World Health Organization Drug Dictionary |

### 1.4. Clinical Assessments

### 1.4.1. Clinical Deterioration and Rescue Therapy

For this protocol, Clinical Deterioration is defined as follows:
Subjects who experience an MG crisis, which is defined as weakness due to MG that is severe enough to necessitate intubation or to delay extubation following surgery; or,
Significant symptomatic worsening that requires rescue medication in the opinion of the Investigator; or,
Subjects for whom the treating physician believes that the subject's health is in jeopardy if rescue therapy is not given (e.g., emergency situations).

Allowed rescue therapy for clinical deterioration includes but is not limited to high-dose corticosteroids, PE, or IVIg and is at the discretion of the Investigator. Plasma exchange was not allowed for prophylaxis or routine maintenance. Every effort was made to notify the Sponsor within 24 hours of administration of rescue therapy.

### 1.4.2. Clinical Evaluation

The Clinical Evaluators are study staff that have been trained and certified in administering the QMG, MG-ADL, and MGC. The Clinical Evaluator may be a neurologist, physical therapist, or other study team member delegated by the Investigator. Clinical Evaluator training and certification for this protocol took place either at the Investigator's meeting or via the Sponsor's designated online training portal or other mechanism.

### 1.4.3. Responsibilities for Myasthenia Gravis Assessments

Responsibilities for MG assessments are listed in Table 15. Throughout the trial, MG assessments was performed at approximately the same time of day by a properly trained evaluator, preferably the same evaluator.

TABLE 15
RESPONSIBILITIES FOR MG ASSESSMENTS

| Assessment | Evaluator |
|---|---|
| MG-ADL | Clinical Evaluator |
| QMG including FVC | Clinical Evaluator |
| MGC (non-MMT Components) | Clinical Evaluator |
| MGC (MMT Components: neck flexion or extension, | PI or Neurologist |
| shoulder abduction, and hip flexion) | |
| MGFA-PIS | PI or Neurologist |
| MGFA Classification | PI or Neurologist |

Abbreviations:
FVC = forced vital capacity;
MG-ADL = Myasthenia Gravis Activities of Daily Living Profile;
MGC = Myasthenia Gravis Composite;
MGFA = Myasthenia Gravis Foundation of America;
MGFA-PIS = Myasthenia Gravis Foundation of America Post-Intervention Status;
MMT = manual muscle test;
PI = Principal Investigator;
QMG = Quantitative Myasthenia Gravis

### 1.5. Study Population

### 1.5.1. Number of Subjects

At least 12 eligible refractory pediatric gMG patients 12 to <18 years of age were enrolled to receive open-label eculizumab infusion in order to obtain at least 10 evaluable patients aged 12 to <18 years for the primary endpoint taking into account potential dropouts. Additional patients between the ages of 6 and 12 may be enrolled but will not be included in the primary analysis. The number of eligible refractory pediatric gMG patients aged 12 to <18 entering on maintenance IVIg treatment in this study was capped at 4 patients.

After 6 patients complete their Week 26 assessments, if the observed standard deviation in change in QMG is 8 or higher, the final sample size will be re-estimated to be at least 14 instead of 12 to preserve adequate power for testing the primary and key secondary endpoints. Patients were eligible to be included in the study only if they satisfy all of the following inclusion/exclusion criteria. The Sponsor's Medical Monitor must approve enrollment for each eligible patient.

Prospective approval of protocol deviations to recruitment and enrollment criteria, also known as protocol waivers or exemptions, was not permitted.

### 1.5.2. Subject Inclusion Criteria

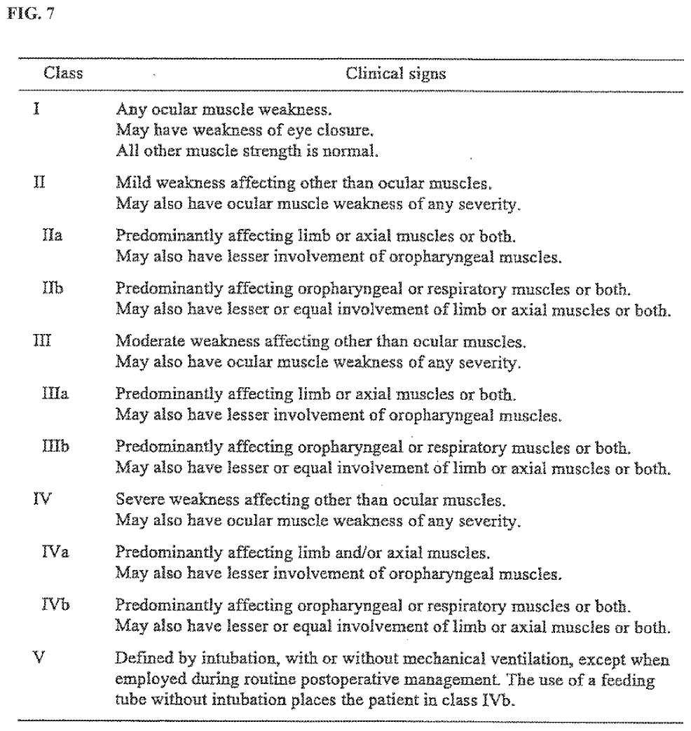

1. Male or female pediatric patients 6 to <18 years of age at time of assent/consent.
2. Patient's legal guardian must be willing and able to give written informed permission and the patient must be willing to give written informed assent (if applicable as determined by the central or local Institutional Review Board [IRB]/Institutional [or Independent] Ethics Committee [IEC]) and comply with the study visit schedule.
3. Parent or other legal guardian must be willing to comply with study requirements for the duration of the study.
4. Must be vaccinated against N meningitidis if not already vaccinated within the time-period of active coverage specified by the vaccine manufacturer, or vaccinate according to current medical/country guidelines at least 2 weeks prior to receiving the first dose of study drug. Patients who require vaccination against N meningitidis will be vaccinated according to current medical/country guidelines; if the vaccine is given less than 2 weeks prior to the first dose of study drug the patient must receive appropriate prophylactic antibiotics until 2 weeks after the vaccination. Patients who cannot be vaccinated must receive antibiotic prophylaxis for the entire treatment period and for 5 months following the last dose of eculizumab.
5. Documented vaccination against H influenzae and S pneumoniae infections prior to dosing as per local and country specific immunization guidelines for the appropriate age group.
6. Diagnosis of MG confirmed by positive serologic test for anti-AChR-Ab at Screening, and one of the following:
   a. History of abnormal neuromuscular transmission test demonstrated by single-fiber electromyography or repetitive nerve stimulation, or
   b. History of positive anticholinesterase test (e.g., edrophonium chloride or neostigmine test), or
   c. Patient demonstrated improvement in MG signs on oral AChIs, as assessed by the Investigator.
7. Presence of refractory gMG, defined as patients with gMG who have one or more of the following:
   a. Failed treatment ≥1 year with at least 1 IST, defined as:
      i. Persistent weakness with impairment of activities of daily living, or
      ii. Myasthenia gravis exacerbation and/or crisis while on treatment, or
      iii. Intolerance to ISTs due to side effect or comorbid condition(s).
   Immunosuppressants include, but are not limited to, corticosteroids, AZA, MMF, methotrexate (MTX), cyclosporine, tacrolimus, or cyclophosphamide.
   b. Require maintenance plasma exchange (PE) or IVIg to control symptoms (i.e., patients who require PE or IVIg on a regular basis for the management of muscle weakness at least every 3 months over the last 12 months prior to Screening).
   c. In the opinion of the Investigator, MG poses a significant functional burden despite treatment.
8. Myasthenia Gravis Foundation of America (MGFA) Clinical Classification of Class II to IV at Screening (FIG. 7).
9. QMG total score ≥12 at Screening.
10. All MG-specific treatment is on a stable dosing regimen of adequate duration prior to Screening as follows:
   a. If patients who enter the study are receiving AZA, they must have been on AZA for ≥6 months and have been on a stable dose for ≥2 months prior to Screening.
   b. If patients who enter the study are receiving other ISTs (i.e., MMF, MTX, cyclosporine, tacrolimus, or cyclophosphamide), they must have been on the IST for ≥3 months and have been on a stable dose for 4 weeks prior to Screening.
   c. If patients who enter the study are receiving maintenance IVIg at Screening, they must have been on maintenance IVIg for at least 12 months and on a stable dose for ≥3 months prior to Screening, with the frequency and dose expected to remain stable during Screening and for 12 weeks following the first dose of study drug. Note: A maximum of 4 patients on maintenance IVIg aged 12 to <18 years are eligible to be enrolled in the study. All other patients aged 12 to <18 years must not have received maintenance IVIg within 3 months of Screening.
   d. If patients who enter the study are receiving oral corticosteroids, they must have been on a stable dose for ≥4 weeks prior to Screening.
   e. If patients who enter the study are receiving a cholinesterase inhibitor, they must have been on a stable dose for 2 weeks prior to Screening.
   f. Female patients of childbearing potential (i.e., have achieved menarche) and male patients with female partners of childbearing potential must follow protocol-specified guidance for avoiding pregnancy while on treatment and for 5 months after the last dose of study drug.
   g. Male patients with a female spouse/partner of childbearing potential or a pregnant or breastfeeding spouse or partner must agree to use double barrier contraception (male condom plus appropriate barrier method for the female partner) while on treatment and for at least 5 months after the last dose of study drug.

### 1.5.3. Subject Exclusion Criteria

1. History of thymoma or other neoplasms of the thymus.
2. History of thymectomy within 12 months prior to Screening.
3. Weakness only affecting ocular or periocular muscles (MGFA Class I).
4. Myasthenia Gravis crisis or impending crisis at or during Screening (MGFA Class V).
5. Are pregnant or lactating.
6. Any unresolved acute, or chronic, systemic bacterial or other infection, which is clinically significant in the opinion of the Investigator and has not been treated with appropriate antibiotics.
7. Unresolved meningococcal infection.
8. Use of PE within 4 weeks prior to first dose.
9. Use of rituximab within 6 months prior to first dose.
10. Participation in another interventional treatment study or use of any experimental therapy within 30 days before initiation of study drug on Day 1 in this study or within 5 half-lives of that investigational product, whichever is greater.
11. Have previously received treatment with eculizumab or other complement inhibitors.
12. Hypersensitivity to murine proteins or to one of the excipients of eculizumab.
13. Any medical or psychological condition that, in the opinion of the Investigator, might interfere with the patient's participation in the study, poses any added risk for the patient, or confounds the assessment of the patient.
14. Known or suspected history of drug or alcohol abuse or dependence within 1 year prior to the start of Screening.

### 1.5.4. Rationale for Inclusion or Exclusion

The inclusion/exclusion criteria in Study ECU-MG-303 have been selected to characterize a pediatric population. Efficacy, safety, and PK/PD data obtained from this study is designed to inform the dose regimen and efficacy and safety profile of eculizumab in pediatric refractory gMG patients.

### 1.6. Discontinuations

### 1.6.1. Discontinuation of Patients

The criteria for enrollment must be followed explicitly. If the investigational site identifies a patient who did not meet enrollment criteria and who was inadvertently enrolled, the Sponsor must be notified. If the Sponsor identifies a patient who did not meet enrollment criteria and who was inadvertently enrolled, the investigational site will be notified. A discussion must occur between the Sponsor and the Investigator to determine whether the patient may continue in the study, with or without study drug. Inadvertently enrolled patients may be maintained in the study and on study drug when the Sponsor agrees with the Investigator that it is medically appropriate for that patient. The Investigator must obtain documented approval from the Sponsor to allow the inadvertently enrolled patient to continue in the study with or without study drug.

Patients will be permanently discontinued from the study in the following circumstances:
Enrollment in any other clinical study involving an investigational product or enrollment in any other type of medical research judged not to be scientifically or medically compatible with this study
Any of the following occur during the study:
   Serious hypersensitive reactions (e.g., anaphylaxis, bronchospasm with wheezing or requiring ventilator support or symptomatic hypotension, clinical syndrome suggestive of serum sickness, vasculitis)
   Severe uncontrolled infection
   Pregnancy or planned pregnancy
Adverse Event
   If the Investigator decides that the patient should be withdrawn because of a serious adverse event (SAE) or a clinically significant laboratory value, the study drug is to be discontinued and appropriate measures are to be taken. The Sponsor or its designee is to be alerted immediately.
Investigator Decision [Physician Decision]
   The Investigator decides that the patient should be discontinued from the study in the best interest of the patient.
Patient Decision [Withdrawal by Patient or Withdrawal by Parent/Guardian]
   The patient or the patient's legal representative (i.e., parents or legal guardian) requests to be withdrawn from the study
Sponsor Decision
   The Sponsor or Health Authority may terminate the study for reasonable cause. Conditions that may warrant termination of the study include, but are not limited to:
      Discovery of an unexpected, serious, or unacceptable risk to patients enrolled in the study
      Sponsor decision to suspend or discontinue testing, evaluation, or development of the study drug
      Failure of the Investigator to comply with the approved protocol, pertinent guideline and/or regulations
      Submission of knowingly false information from the Investigator to the Sponsor and/or regulatory authorities
      The Sponsor medical monitor deems it is in the best interest of the patient

Should the study be terminated early, the Sponsor will notify the national competent authority and the IRB or IEC according to local requirements.

Patients who discontinued the study early had an ET visit at the time of withdrawal and a Follow-up Visit at 8 weeks following the last eculizumab dose performed, as shown in the Schedule of Assessments. If a female patient is permanently discontinued from eculizumab treatment due to pregnancy, the Investigator will attempt to follow-up with the patient until the outcome of the pregnancy is established.

### 2. Study Drug Materials and Management

### 2.1. Investigational Product and Labeling

The study drug, eculizumab, was manufactured and supplied by Alexion or a contract manufacturing organization in single 30 mL vials as a solution concentration of 10 mg/mL Each vial contains 300 mg of eculizumab for intravenous (IV) administration. Eculizumab was individually packaged in kits. Both vials and kits were labeled according to the protocol and local regulatory requirements. Study drug orders were released to each site upon receipt of all required documents based upon applicable regulations.

TABLE 16
STUDY DRUG

| | |
|---|---|
| Product Name | Eculizumab |
| Dosage Form | Concentrate for solution for infusion |
| Unit Dose | 300 mg |
| Route of Administration | Intravenous infusion |
| Physical Description | 30 mL vial |
| Manufacturer | Alexion or a contract manufacturing |
| | organization |

### 2.2. Study Drug Storage

Upon arrival at the center, the study drug should be promptly removed from the shipping cooler and stored in refrigerated conditions between 2° C. to 8° C. The study drug must be stored in a secure, limited-access storage area, and temperature must be monitored daily. On-site storage temperature excursions must be reported to the Sponsor in a timely manner.

Diluted solutions of study drug (dosing solutions) may be stored between 2° C. to 8° C. (36° F. to 46° F.) and at room temperature until the end of study drug infusion for a maximum of 24 hours. The 24-hour expiration includes preparation time, storage time, warming time, and infusion time. If the study drug is prepared more than 4 hours in advance of a patient's visit, the diluted material should be stored between 2° C. to 8° C. The solution should be allowed to warm to room temperature prior to administration. The material must not be heated (e.g., by using a microwave or other heat source) other than by ambient air temperature.

### 2.3. Study Drug Preparation

Infusions of study drug should be prepared using aseptic technique. Each vial of study drug contains 300 mg of active ingredient in 30 mL of product solution. Withdraw the required amount of study drug from the vials. Transfer the recommended dose to an infusion bag. Dilute the study drug to a final concentration of 5 mg/mL by addition to the infusion bag of the appropriate amount (equal volume) of 0.9% Sodium Chloride Injection, USP; 0.45% Sodium Chloride Injection, USP; 5% Dextrose in Water Injection, USP; or Ringer's Injection, USP. The final volume of a 5 mg/mL diluted study drug solution is 60 mL for 300 mg doses (1 vial), 120 mL for 600 mg doses (2 vials), 180 mL for 900 mg doses (3 vials), and 240 mL for 1200 mg doses (4 vials).

TABLE 17
STUDY DRUG RECONSTITUTION

| | Volume of | Volume of | Total Volume of |
|---|---|---|---|
| Study Drug | Study Drug | Diluent^{a} | Administration |
| 300 mg (1 vial) | 30 mL | 30 mL | 60 mL |
| 600 mg (2 vials) | 60 mL | 60 mL | 120 mL |
| 900 mg (3 vials) | 90 mL | 90 mL | 180 mL |
| 1200 mg (4 vials) | 120 mL | 120 mL | 240 mL |

Choose one of the following diluents: 1) 0.9% sodium chloride; 2) 0.45% sodium chloride; 3) 5% dextrose in water; 4) Ringer's injection.

Gently invert the infusion bag containing the diluted study drug solution to ensure thorough mixing of the product and diluents. Discard any unused portion left in a vial, as the product contains no preservatives. The diluted solution should be allowed to warm to room temperature by exposure to ambient air prior to administration.

### 3. Study Drug Administration

Prior to study drug administration, the diluted solution should be allowed to warm to room temperature by exposure to ambient air. The diluted solution must not be heated in a microwave or with any heat source other than ambient air temperature. Parenteral drug products should be inspected visually for particulate matter and discoloration prior to administration.

The diluted study drug should be intravenously administered via IV infusions, using a weight-based schedule, over 1 to 4 hours. It is not necessary to protect the infusion bags from light while study drug is being administered to the patient. The patient should be monitored for at least 1 hour following infusion.

If an AE occurs during administration of the study drug, the infusion may be slowed or stopped at the discretion of the Investigator, depending upon the nature and severity of the event; however, the overall duration should not exceed 2 hours from the start of the infusion in adolescents ≥12 years of age and 4 hours from the start of infusion in children <12 years of age. The AE must be captured in the patient's source document and eCRF.

The actual start and stop times of all dose administrations will be recorded in the patient's source documents and eCRF.

Sites must have resuscitation equipment, emergency drugs, and appropriately trained staff available during the infusion, and for at least 1 hour after patients have completed their infusion.

### 3.1 Dosing Regimens

### 3.1.1. Dosing Regimen Preparation and Administration

Eculizumab was administered weekly during the initial induction phase and every 2 weeks during the maintenance phase. The dosing regimen was based on the pediatric patient's body weight (Table 18).

TABLE 18
WEIGHT-BASED DOSING REGIMEN OF ECULIZUMAB

| Weight Cohort^{a} | Induction Phase | Maintenance Phase |
|---|---|---|
| ≥40 kg | 900 mg weekly x | 1200 mg at Week 4; |
| | 4 doses | then every 2 weeks |
| 30 to <40 kg | 600 mg weekly x | 900 mg at Week 2; |
| | 2 doses | then every 2 weeks |
| 20 to <30 kg | 600 mg weekly x | 600 mg at Week 2; |
| | 2 doses | then every 2 weeks |
| 10 to <20 kg | 600 mg weekly x | 300 mg at Week 1; |
| | 1 dose | then every 2 weeks |

^{a}Based on the most recently recorded body weight at a prior dosing visit

Eculizumab 300 mg, 600 mg, 900 mg, or 1200 mg was administered via IV infusion based on the patient's most recently recorded body weight at a prior dosing visit, as presented in Table 18. Doses of study drug was prepared and dispensed by qualified study personnel. Study drug was dispensed only to enrolled patients who are confirmed eligible for participation in this study. Once study drug was prepared for a patient, it was only administered to that patient. Vials of study drug are for one-time use only, and any drug product remaining in the vial should not be used for another patient. Any drug remaining in the infusion tubing or infusion bag should not be used for another patient.

### 3.1.2. Supplemental Eculizumab Doses in Patients Receiving Maintenance IVIg Treatment

Maintenance IVIg treatment may interfere with the endosomal neonatal Fc receptor (FcRn) recycling mechanism of monoclonal antibodies and, thus, may decrease serum eculizumab concentrations (22, 23, 24). Therefore, for pediatric gMG patients receiving maintenance IVIg treatment, a series of supplemental doses of eculizumab will be administered to account for the anticipated approximately 50% increase in eculizumab clearance. For further details on supplemental dosing, please refer to Table 19. In addition, patients are to continue eculizumab infusion according to the protocol-specified dosing regimen.

TABLE 19
SUPPLEMENTAL DOSING REGIMEN OF ECULIZUMAB
IN PATIENTS RECEIVING MAINTENANCE IVIG

| Weight Cohort^{a} | Induction Phase | Maintenance Phase |
|---|---|---|
| ≥40 kg | 600 mg | 600 mg |
| 30 to <40 kg | 300 mg | 600 mg |
| 20 to <30 kg | 300 mg | 300 mg |
| 10 to <20 kg | 300 mg | 300 mg |

^{a}Based on the most recently recorded body weight at a prior dosing visit

Notes: The timing of supplemental eculizumab dosing varies by IVIg frequency and is provided below:
If a patient continues to receive IVIg at a dose interval more frequent than every 4 weeks during eculizumab treatment, a supplemental dose will be administered at the same time that each scheduled dose of eculizumab is administered.
If a patient receives IVIg treatment at a dose interval less frequent than every 4 weeks during eculizumab treatment, a supplemental dose will be administered following the last dose of IVIg infusion cycle at the next scheduled eculizumab dose.
If a patient receives IVIg treatment within 4 weeks prior to receiving the first dose of eculizumab, a supplemental dose of eculizumab will be administered at the same time that the first dose of eculizumab is administered (i.e., the total dose is the supplemental dose plus the first scheduled dose).

For patients who enter the study on a stable IVIg maintenance dose regimen, PK/PD samples will be analyzed after the first 4 weeks of eculizumab administration for the evaluation of eculizumab exposure. If the PK concentration values are greater than 1790 μg/mL, the supplemental dose of eculizumab may be adjusted downward or waived, as appropriate, so that the predicted maximum PK concentration would be below 1790 μg/mL

### 3.1.3. Supplemental Eculizumab Doses Following Rescue Therapy

When IVIg is administered as acute rescue therapy for clinical deterioration, no supplemental dose of eculizumab should be administered. However, if a patient receives more than 1 dose of IVIg as rescue therapy within a 12-week period, supplemental eculizumab should be administered at the time of the second dose and at each subsequent IVIg dose within the 12-week period in accordance with Table 19.

If a patient undergoes PP/PE/FFP for clinical deterioration during the study, a supplemental dose of study drug must be administered within 1 to 2 hours after each PP/PE/FFP session unless the PP/PE/FFP session is on the day of a scheduled study drug infusion. If FFP has been administered, a supplemental dose of study drug must be administered 1 hour prior to each infusion of FFP. If the PP/PE/FFP is on the day of a scheduled study drug infusion, the scheduled dose of study drug (instead of the supplemental dose) should be administered within 1 to 2 hours after the completion of PP/PE/FFP session. For further details on supplemental dosing please refer to Table 20. In addition, patients are to continue eculizumab infusion according to the protocol specified dosing regimen.

TABLE 20
SUPPLEMENTAL DOSING REGIMEN OF ECULIZUMAB
FOR PLASMA EXCHANGE/PLASMA INFUSION

| | | Supplemental | |
|---|---|---|---|
| | Most | Eculizumab Dose With | Timing of |
| | Recent | Each Plasma Exchange/ | Supplemental |
| Type of | Eculizumab | Plasma Infusion | Eculizumab |
| Intervention | Dose | Intervention | dose |
| Plasmapheresis | 300 mg | 300 mg per each | Within 1 to 2 |
| or plasma | | plasmapheresis or | hours after |
| exchange | | plasma exchange | each |
| | | session | plasmapheresis |
| | 600 mg or | 600 mg per each | or plasma |
| | more | plasmapheresis or | exchange |
| | | plasma exchange | |
| | | session | |
| Fresh frozen | 300 mg or | 300 mg per infusion | Approximately |
| plasma | more | of fresh frozen | 60 minutes^{a} |
| infusion | | plasma | prior to each |
| | | | infusion of |
| | | | fresh frozen |
| | | | plasma |

^{a}Supplemental dosing of eculizumab should occur 60 ± 15 minutes prior to each infusion of fresh frozen plasma.

### 3.2. Concomitant Medications

### 3.2.1. Allowed Medications

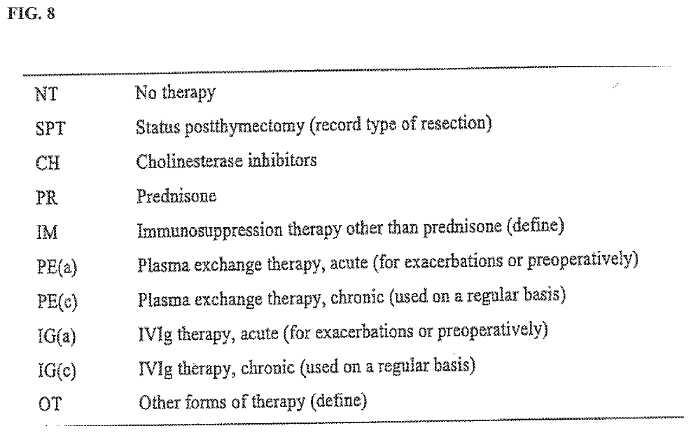

Palliative and supportive care is permitted during the course of the study for underlying conditions. Patients may continue to receive ACh1, IVIg, and ISTs during the study where applicable under certain restrictions. A schematic of the commonly-used MGFA therapy status is shown in FIG. 8. For patients who enter the study receiving any background therapy, the dose/schedule may not be changed during the Primary Evaluation Treatment Period before Week 12, unless deemed necessary per the Investigator based on clinical safety evaluation and if Sponsor approval is obtained. Dose change with background medication is permitted after Week 12 at the Investigator's discretion and with Sponsor notification.

During the Extension Period, changes in background medications will be permitted at the Investigator's discretion and with Sponsor notification.

Changes in concomitant medications and/or nondrug therapies and procedures will be recorded in the eCRF.

The following additional restrictions apply:
Acetylcholinesterase inhibitors
   Acetylcholinesterase inhibitor treatment must be withheld for at least 10 hours prior to administration of the QMG and MGC tests.
Immunosuppressive therapies:
   The following ISTs are allowed during the study: corticosteroid, AZA, MMF, MTX, tacrolimus, cyclosporine, or cyclophosphamide. The ISTs and the appropriate dose levels to be used for an individual patient will be at the discretion of the treating physician.
   High-dose steroid should be reserved for patients that experience clinical deterioration as defined by this protocol. Every effort should be made to notify the Sponsor within 24 hours of administration should a patient require a rescue therapy for clinical deterioration.
Intravenous immunoglobulin
   If a patient enters the study receiving maintenance IVIg, a supplemental dose of study drug will be administered at the first scheduled dose.
Plasma Exchange/Plasmapheresis/Fresh Frozen Plasma (PE/PP/FFP)
   Use of PE/PP/FFP will be allowed as rescue therapy for patients who experience a clinical deterioration as defined by this protocol. The rescue therapy used for an individual patient will be at the discretion of the treating physician. Every effort should be made to notify the Sponsor within 24 hours should a patient require a rescue therapy.
   If a patient undergoes PE during the study, a supplemental dose of study drug must be administered.

### 3.2.2. Disallowed Medications

The use of rituximab is prohibited during the study.

### 3.2.3. Vaccination

Patients must be vaccinated against N meningitidis if not already vaccinated within the time period of active coverage specified by the vaccine manufacturer, or vaccinated according to current medical/country guidelines at least 2 weeks prior to receiving the first dose of study drug. If the vaccine is to be administered within 2 weeks prior to the first dose of study drug, patients must receive appropriate prophylactic antibiotics until 2 weeks after vaccination. Patients must remain within the vaccine manufacturer's specified period of active coverage during study participation and for 5 months following the last dose of eculizumab.

Vaccines against serotypes A, C, Y, W135, and B, where available, are recommended to prevent common pathogenic meningococcal serotypes.

Patients who cannot be vaccinated must receive antibiotic prophylaxis for the entire treatment period and for 5 months following the last dose of eculizumab.

In addition to meningococcal vaccination, patients must be vaccinated against H influenzae and S pneumoniae, and strictly adhere to the national vaccination recommendations for each age group.

Due to the length of the Extension Period of this study, patients may be revaccinated for appropriate vaccinations based on adherence to the national vaccination recommendations for each age group to provide active coverage as specified by the vaccine manufacturer or according to current medical/country guidelines. Investigators will assess the need for revaccination, which will be recorded in the source documents and electronic case report form (eCRF).

### 3.3. Treatment Compliance

The infusion of study drug into patients will be under the supervision of the Investigator or their designee to ensure that the patients received the appropriate dose at the appropriate time-points during the study.

Patients who fail to return for a scheduled visit within the acceptable visit windows (±2 days) must be contacted by the site study staff to determine the reason for missing the appointment. Patients should be strongly encouraged to return to the investigational site for evaluation if clinical deterioration or an AE is suspected to have occurred. In the exceptional circumstance, if a patient cannot or does not come to the study site for examination, the patient will be instructed to see his or her local neurologist or physician. In this event, the investigational site will (or attempt to) obtain relevant medical records as documentation from the local physician's examination, and enter relevant data in the eCRF as appropriate.

As it is vital to obtain information on any patient's missing visit (in-clinic or remote) to assure the missing appointment was not due to a clinical deterioration or an AE, every effort must be made to undertake protocol-specified follow-up procedures (Table 7 and Table 9 based on weight cohort). Follow-up due-diligence documentation will consist of 3 phone calls followed by 1 registered letter to the patient's last known address, and documented in both the source documents and the eCRF.

Patients should be registered in the IXRS as soon as the Study ECU-MG-303 informed consent form (ICF) is signed. The initial shipment of study drug for Study ECU-MG-303 will be triggered by the IXRS.

### 3.4. Continued Access to Study Drug

After completing the 26-week Primary Evaluation Treatment Period, patients will continue receiving eculizumab in the Extension Period for up to additional 208 weeks.

### 4.0. Assessment of Efficacy

Efficacy assessments will be performed as summarized in the Schedules of Assessments. Preferably, the same parent or guardian is recommended to be available to accompany the child to each visit in order to reduce variability in endpoint reporting.

### 4.1. Quantitative Myasthenia Gravis (QMG) Score

The QMG scoring system consists of 13 items: ocular (2 items), facial (1 item), bulbar (2 items), gross motor (6 items), axial (1 item), and respiratory (1 item); each graded 0 to 3, with 3 being the most severe (Table 2). The range of total QMG score is 0 to 39. The QMG scoring system is considered to be an objective evaluation of therapy for MG and is based on quantitative testing of sentinel muscle groups. The MGFA task force has recommended that the QMG score be used in prospective studies of therapy for MG.

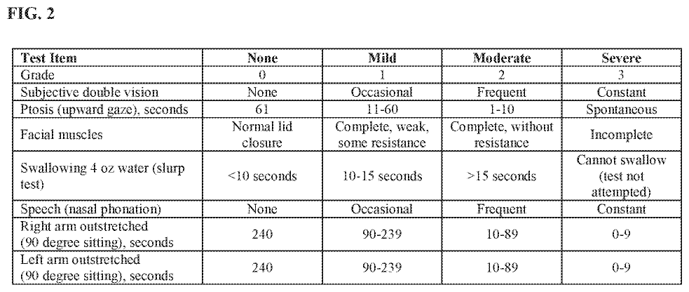

A modified QMG score has been developed for use in patients younger than 12 years of age that will be used for patients aged 6 to 11 years at the time of Screening (25; FIG. 2). The modified QMG omits the assessment of grip strength and uses a modified assessment of swallowing (slurp test) compared to the traditional QMG, with total scores ranging from 0 to 21. In this study, patients will continue to be evaluated based on the QMG scale initially completed upon entry into the study. Change in age during the study will not constitute a patient changing the type of survey completed (i.e., a patient who enrolls at age 11 will continue being assessed with the modified QMG scale even after he or she reaches 12 years of age). The QMG assessment will be administered at the protocol-specified time points at approximately the same time of day by a properly trained evaluator, preferably the same evaluator, throughout the study.

### 4.2. Myasthenia Gravis Activities of Daily Living (MG-ADL) Score

The MG-ADL is an 8-point questionnaire that focuses on relevant symptoms and functional performance of activities of daily living in MG patients (Table 1). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0 to 24. The recall period for MG-ADL is the preceding 7 days or since the last visit if the visit interval is less than 7 days. The MG-ADL assessment will be administered at the protocol-specified time points at approximately the same time of day by a properly trained evaluator, preferably the same evaluator, throughout the study.

For patients <12 years of age, caregiver assistance can be provided during the MG-ADL assessment.

### 4.3. Myasthenia Gravis Composite (MGC) Score

The MGC is a validated assessment tool for measuring clinical status of patients with MG (16). The MGC assesses 10 important functional areas most frequently affected by MG, and the scales are weighted for clinical significance that incorporates patient-reported outcomes (Table 3). The range of total MGC score is 0 to 50. Higher scores indicate more functional impairment. In this study, the MGC assessment will be administered at the protocol-specified time points at approximately the same time of day by a properly trained evaluator, preferably the same evaluator, throughout the study.

For patients <12 years of age, caregiver assistance can be provided during the MG-ADL assessment.

### 4.4. Myasthenia Gravis Foundation of America (MGFA) Post-Intervention Status

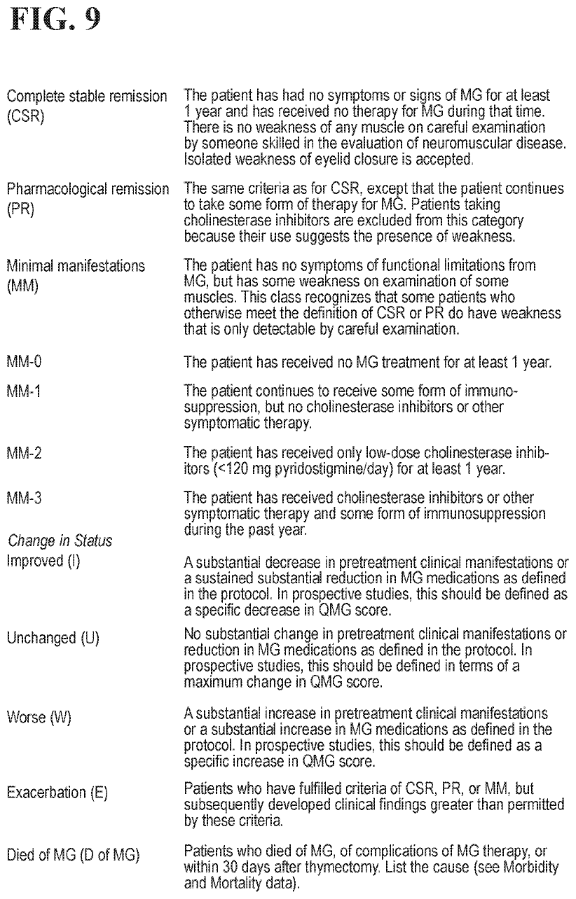

The MG clinical state will be assessed using the MGFA Post-Intervention Status. Change in status categories of Improved, Unchanged, Worse, as well as the Minimal Manifestation will be assessed by the PI or the same neurologist skilled in the evaluation of MG patients throughout the study (FIG. 9).

### 4.5. European Quality of Life 5-Dimension (EQ-5D-Y)

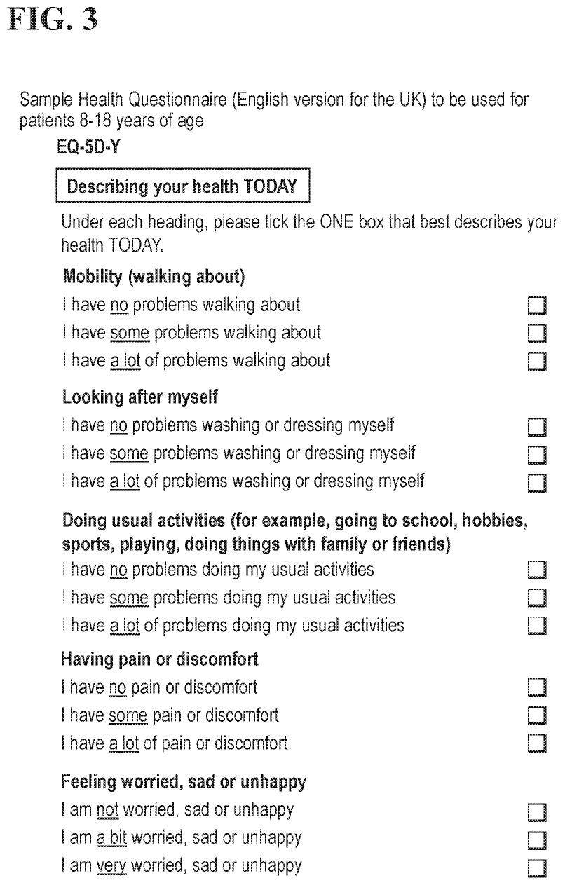

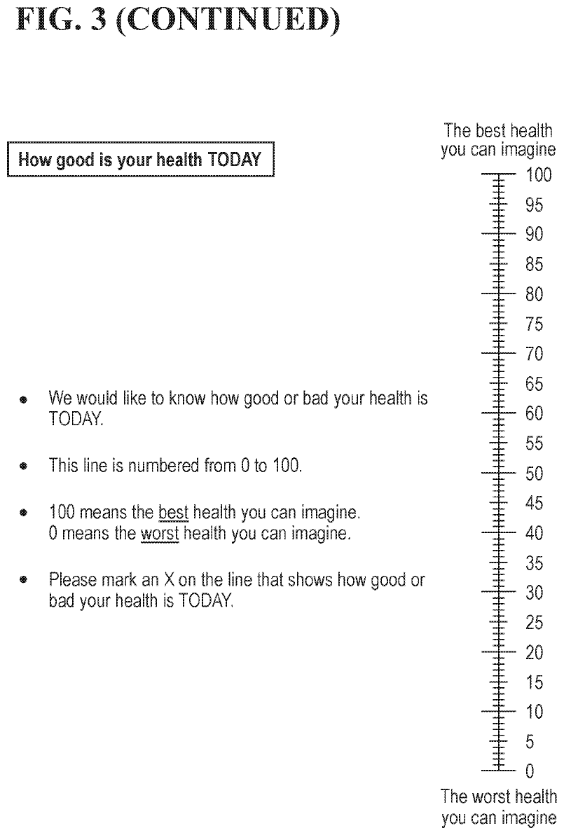

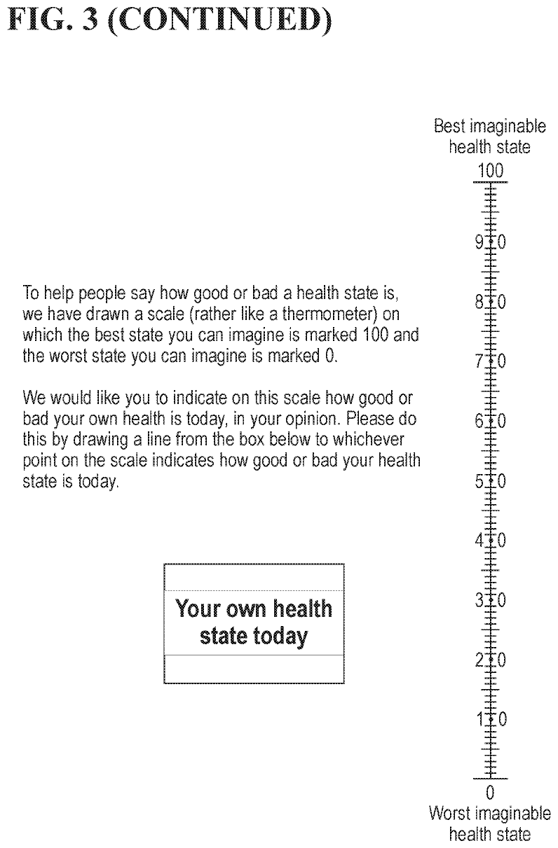

The EQ-5D-Y (FIG. 3) is a reliable and validated survey of health status in 5 areas: mobility, self-care, usual activities, pain/discomfort, and anxiety/depression, each of which is completed by the patient for patients 212 years of age (at time of assessment) and completed by the patient's caregiver or with caregiver assistance for patients <12 years of age (26). Each area has 3 levels: Level 1 (no problems), Level 2 (some problems), and Level 3 (extreme problems). The EQ visual analogue scale (VAS) records the patient's self-rated health on a vertical, 20 cm VAS where the endpoints are labeled ‘Best imaginable health state, marked as 100’ and ‘Worst imaginable health state, marked as 0’. Patients will continue to be evaluated based on the survey initially completed upon entry into the study.

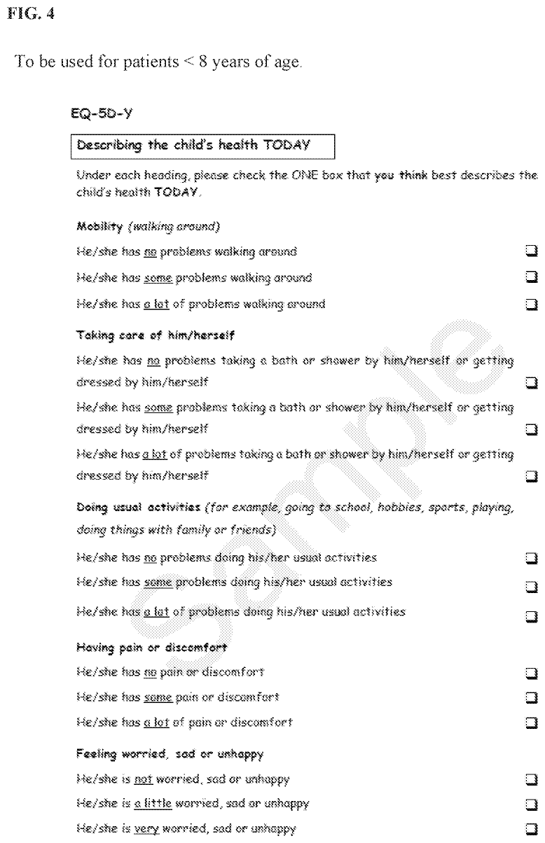

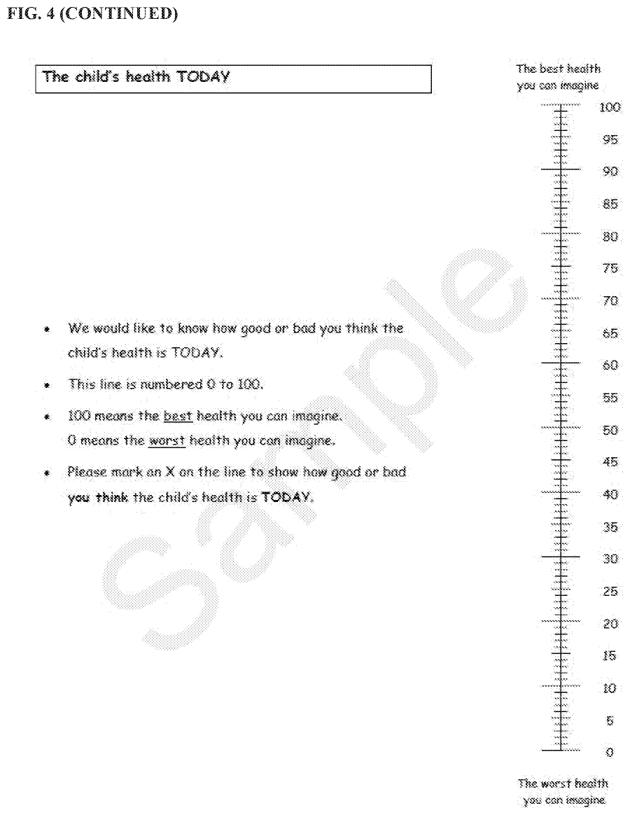

Change in age during the study will not constitute a patient changing the type of survey completed. Patients who are younger than the lowest age range of the survey (i.e., patients <8 years of age) will be evaluated using the proxy version of the EQ-5D-Y (FIG. 4). The parent or legal guardian (the proxy) will be asked to rate the child's health-related quality of live in their (the proxy's) opinion. The EQ-5D-Y assessment will be administered at the protocol-specified time points at approximately the same time of day throughout the study.

### 4.6. Neurological Quality of Life Fatigue (Neuro-QoL Fatigue) Questionnaire

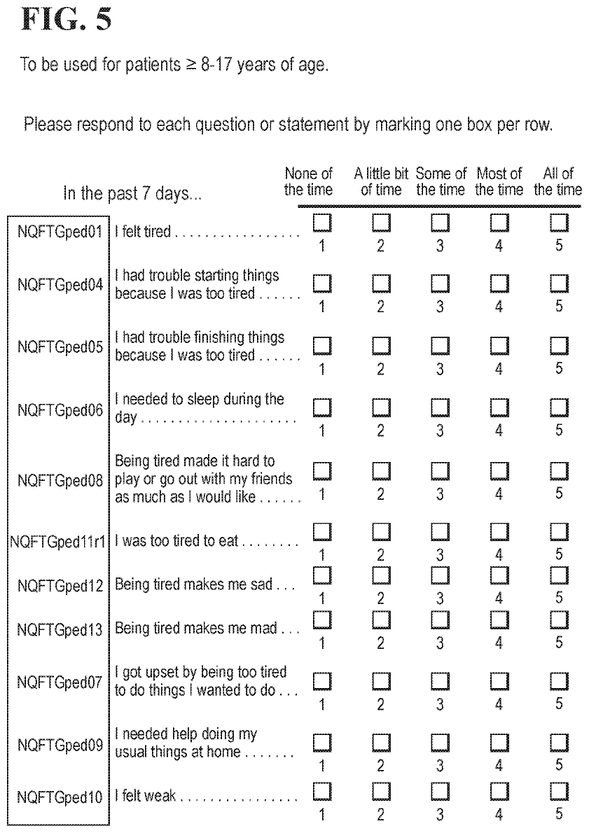

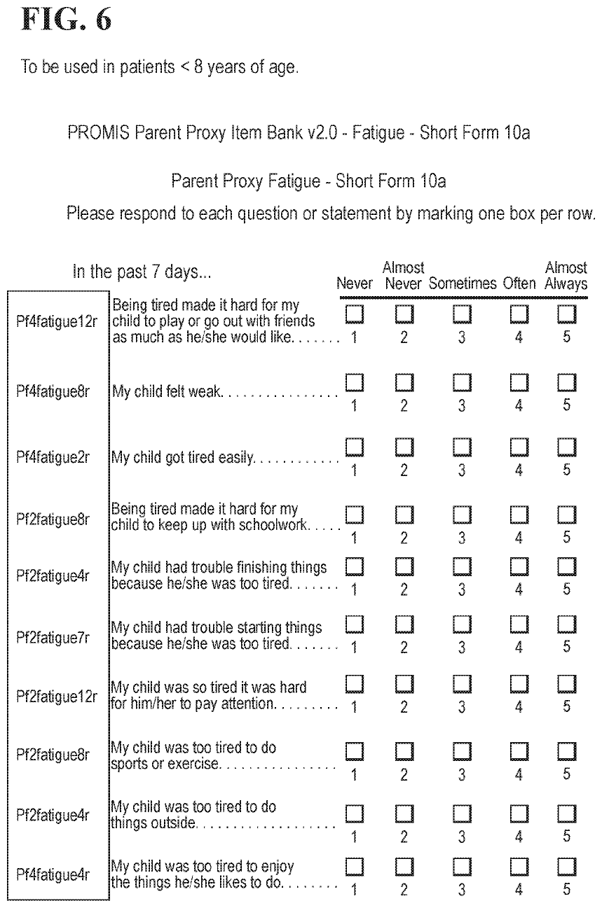

The Neuro-QoL Pediatric Fatigue questionnaire (FIG. 5) is a reliable and validated brief 11-item survey of fatigue, completed by the patient for patients ≥12 years of age (at time of assessment) and completed by the patient's caregiver or with caregiver assistance for patients <12 years of age (18). Higher scores indicate greater fatigue and greater impact of MG on activities. Patients will continue to be evaluated based on the survey initially completed upon entry into the study. Change in age during the study will not constitute a patient changing the type of survey completed. Patients who are younger than the lowest age range of the applicable scale (i.e., patients <8 years of age) will be evaluated using the proxy version (short form) of the Neuro-QoL Pediatric Fatigue questionnaire (FIG. 6). The parent or legal guardian (the proxy) will complete the measure on the child's behalf following administration of these instructions: “The following questionnaires will ask about your child's symptoms and activity levels; his/her ability to think, concentrate and remember things; questions specific to his/her condition, and questions related to his/her quality of life. Please answer the following questions based on what you think your child would say.” The Neuro-QoL Pediatric Fatigue questionnaire will be administered at the protocol-specified time points at approximately the same time of day throughout the study.

### 4.7. Myasthenia Gravis Quality of Life (MG-QoL) 15

The 15-item Myasthenia Gravis Qualify of Life scale (MG-QoL 15) is a health-related quality of life evaluative instrument specific to subjects with MG. MG-QOL 15 was designed to provide information about subjects' perception of impairment and disability and the degree to which disease manifestations are tolerated and to be easy to administer and interpret. The MG-QOL 15 is completed by the subject. Higher scores indicate greater extent of and dissatisfaction with MG-related dysfunction. A clinically meaningful improvement in a patient's MG-QOL 15 would be an increase in score after 26 weeks of treatment.

### 4.8. Other Assessments

### 4.8.1. Myasthenia Gravis Disease Biomarker

Blood samples for assay of the AChR-Ab will be collected at Screening as specified in the Schedules of Assessments.

### 4.8.2. Pharmacokinetics and Pharmacodynamics

Blood samples will be collected at specified time points to study the PK of eculizumab in pediatric patients with refractory gMG. Pharmacokinetic parameters such as maximum concentration and concentration after the first dose, and during the induction and maintenance treatment phase will be obtained. Clearance and terminal half-life will be estimated.

Blood samples for PD analysis will be collected at specified time points to assess pre- and post-treatment serum hemolytic activity and, therefore, C5 complement activity inhibition.

Baseline PK and PD samples will be collected prior to the first dose, and peak samples will be collected 1 hour after the first dose. An intermediate blood sample will also be collected 24 hours after completion of the first dose. Similarly, PK/PD samples will be drawn 1 hour after completion of the first maintenance dose.

Blood samples for PK and PD may be collected within ±15 minutes of the specified time points. The date and exact time of collection must be recorded on the eCRF and the central laboratory requisition form.

Blood samples collected for PK and PD will be kept frozen and stored for a maximum of 5 years after all the specified PK and PD data will have been collected for the study. The frozen samples may be used for future research related to eculizumab. Each sample will be given a code. This code will allow the patient sample to be used without the researchers knowing the patient's name. The results of the research may be presented at scientific meetings or in publications; however, patient identity will not be disclosed. All other blood and urine samples collected during the study will be destroyed after the tests have been completed.

### 5. Statistical Considerations

### 5.1. General Considerations

All summary statistics will be computed and displayed by visit where applicable. Descriptive statistics for continuous variables will minimally include the following: n, mean, standard deviation, minimum, median, and maximum. For categorical variables, frequencies and percentages will be presented. Graphical displays will be provided as appropriate. Unless otherwise stated, all statistical summaries will be displayed by younger (<12 years) and older (12 to <18 years) age groups separately and/or combined. A two-sided Type I error of 5% will be used for statistical tests unless otherwise stated. The statistical modeling will be performed for the applicable endpoints only for the older age group, as very few patients are anticipated to be enrolled in the younger age group. Study data will be summarized by each age group and overall. All study data collected on the CRF and from other sources will be listed for individual patients. Baseline will be defined as the last non-missing value on or prior to first dose of eculizumab, unless otherwise stated. No imputation will be performed for missing efficacy data. The statistical analysis plan (SAP) will be developed and finalized prior to the primary analysis and will provide further details.

The primary analysis will be conducted when all patients have completed the 26-week Primary Evaluation Treatment Period or discontinued prior to the completion of the Primary Evaluation Treatment Period. This analysis will include all efficacy, safety, and PK/PD study data for regulatory submission purpose. Outlines the plan for subsequent interim analyses and the final analysis are shown below.

Analyses will be performed using the SAS® software Version 9.4 or higher.

### 5.2. Hypotheses

The null and alternative hypotheses related to primary endpoint for this study is described as:

H₀:μ=0 vs. H₁:μ≠0,

where μ represents the mean improvement in QMG from Baseline over time regardless of rescue under null and alternate hypotheses.

### 5.3. Determination of Sample Size

The sample size will be determined to ensure adequate power for testing the primary endpoint and the key secondary endpoints for this study in the older age group. The assumptions regarding change from Baseline in QMG and MG-ADL scores were based on the subset of a younger patient population treated with eculizumab in Studies ECU-MG-301 and ECU-MG-302.

Based on eleven younger (<25 years) patients from those studies, the mean and SD were calculated at both 12 and 26 weeks of eculizumab exposure (Table 21).

TABLE 21
MEAN [±SD] CHANGE FROM BASELINE IN QMG
AND MG-ADL FOR YOUNGER PATIENTS (DATA FROM STUDIES
ECU-MG-301 AND ECU-MG-302)

| | QMG | MG-ADL |
|---|---|---|
| Baseline | 20.9(6.32) | 8.9(4.46) |
| Change at Week 12 | −8.9(6.86) | −5.7(4.27) |
| Change at Week 26 | −9.0(5.58) | −6.9(4.93) |

Abbreviations:
MG-ADL = Myasthenia Gravis Activities of Daily Living profile;
QMG = Quantitative Myasthenia Gravis score for disease severity

Based on a two-sided one-sample t-test with a significance level of 0.05 and assuming an improvement in QMG total score at Week 26 with mean (SD) of 9 (7), a total of 10 patients will provide approximately 95% power to detect a statistically significant improvement in QMG total score from Baseline. Similarly, assuming an improvement in MG-ADL total score at Week 26 with mean (SD) of 7 (5), a total of 10 patients will provide approximately 97% power. Likewise, based on a two-sided one-sample t-test with a significance level of 0.05 and assuming an improvement in QMG total score at Week 12 with mean (SD) of 9 (7), a total of 10 patients will provide approximately 95% power to detect a statistically significant improvement in QMG total score from Baseline. Similarly, assuming an improvement in MG-ADL total score at Week 12 with mean (SD) of 6 (5), a total of 10 patients will provide approximately 92% power.

Assuming a loss of 15% of patients due to not meeting evaluability criteria, a total of at least 12 patients will be planned to be enrolled in the older age group for the primary analysis to enroll at least 10 evaluable patients. There will be no statistical considerations to determine the number of patients to be enrolled in the younger age group. Additional information about sample size re-estimation is described below.

In addition, a maximum of 4 patients on maintenance IVIg aged 12 to <18 years are eligible to be enrolled in the study. All other patients aged 12 to <18 years must not have received maintenance IVIg within 3 months of Screening.

### 5.4. Interim Monitoring of Variability and Sample Size Re-Assessment

Since the estimates provided in Table 21 are based on a small number of patients, an interim monitoring of the variability in change in QMG from Baseline will be performed. After 6 patients complete the Week 26 assessments, if the observed standard deviation in QMG change from Baseline is approximately 8 or higher, the final sample size will be re-estimated to be at least 14 patients instead of 12 patients to preserve adequate power for testing the primary and key secondary endpoints.

### 5.5. Analysis Sets

### 5.5.1. Full Analysis Set

Efficacy analyses will be performed on the Full Analysis Set (FAS), which consists of all patients who received at least 1 dose of eculizumab. A subset of the FAS that includes older patients (12 to <18 years of age) only will be used for analyses of the primary and key secondary endpoints and will be defined as the modified Full Analysis Set (mFAS). The FAS will be used for other efficacy results summaries by including younger patients (<12 years of age), if any are enrolled.

### 5.5.2. Safety Set

Safety analyses will be performed on the Safety Set, which consists of all patients who received at least 1 dose of eculizumab.

### 5.5.3. Other Analysis Set

Pharmacokinetic/PD analyses will be performed on the PK/PD Analysis Set. The PK/PD Analysis Set will include patients who have PK/PD data assessments during this study.

### 5.6. Demographics and Baseline Characteristics

Patient demographic and baseline characteristics will be summarized for the Safety Set. Summary statistics will be presented. No formal hypothesis testing will be performed.

### 5.7. Patient Disposition

The number of patients screened and the number of patients in different analysis sets will be summarized. The number and percentage of patients discontinued will be summarized along with reasons for discontinuation in the Safety Set.

### 5.8. Prior and Concomitant Medications

Medications will be coded using the World Health Organization Drug Dictionary (WHODrug; the most current version available at the time of the analyses).

### 5.9. Efficacy Analyses

### 5.9.1. Primary Efficacy Analysis

The primary efficacy endpoint is the change from Baseline in the QMG total score over time regardless of rescue treatment. The primary efficacy analysis for the change from Baseline in the QMG total score will be conducted at Week 12 in order to assess the effect of eculizumab treatment during the Primary Evaluation Treatment Period after the 12 weeks during which MG medications (i.e., ISTs, IVIg) are held constant. The Paediatric Committee of the European Medicines Agency (PDCO)-specific primary efficacy analysis will be conducted at Week 26. The primary efficacy analysis will be performed on the mFAS. A Repeated-Measures model will be used to analyze observed change in QMG with baseline QMG score and visits as covariates. The least-squares mean at Week 12 will be used to test the primary hypothesis at a significance level of 5%. The least-squares mean at Week 26 will be used to test the PDCO-specific primary hypothesis at a significance level of 5%. The standard error of the mean and 95% confidence interval will be produced. Missing primary endpoints at post-baseline visits will not be imputed.

5.9.1.1. Analysis of Primary Endpoint based on Evaluable Patients

The analysis of the primary efficacy endpoint will be performed on the mFAS (evaluable) set.

### 5.9.2. Analyses of Secondary Efficacy Endpoints

The secondary efficacy endpoints are:
Change from Baseline in the MG-ADL total score over time regardless of rescue treatment
Proportion of patients with ≥3-point reduction in the MG-ADL total score from Baseline over time with no rescue treatment
Proportion of patients with ≥3-point reduction in the MG-ADL total score from Baseline over time regardless of rescue treatment
Proportion of patients with ≥5-point reduction in the QMG total score from Baseline over time with no rescue treatment
Proportion of patients with ≥5-point reduction in the QMG total score from Baseline over time regardless of rescue treatment
Change from Baseline in the MGC total score over time regardless of rescue treatment
Change from Baseline in EQ-5D-Y over time regardless of rescue treatment
Change from Baseline in Neuro-QoL Pediatric Fatigue over time regardless of rescue treatment
MGFA Post-Interventional Status over time regardless of rescue treatment
Total number and percentage of patients with clinical deteriorations, myasthenic crises, and rescue therapy use over time

For all patients in the mFAS, the secondary endpoints that involve change from Baseline will be analyzed at a particular visit based on the repeated-measures models with effects for the particular baseline covariate and visit. Confidence intervals and p-values will be presented by visit. Graphical displays over time will be produced by visit. Missing secondary endpoint assessments will not be imputed.

For all patients in the mFAS, the secondary endpoints that involve proportion of patients with a pre-specified response will be summarized at a particular visit. Confidence intervals and p-values will be presented by visit.

The number and percentage of patients with at least one on-study clinical deterioration and/or MG crisis during first 26 weeks will be summarized. Use of rescue therapy during the first 26 weeks will also be summarized.

### 5.9.3. Extension Period Efficacy Endpoints

The efficacy endpoints related to the Extension Period are:
Total number and percentage of patients with clinical deteriorations and/or myasthenic crisis during the study
Total number and percentage of patients needing rescue therapy during the study
Change from Baseline in the QMG total score regardless of rescue treatment
Change from Baseline in the MG-ADL total score regardless of rescue treatment
Change from Baseline in the MGC total score regardless of rescue treatment
Change from Baseline in EQ-5D-Y regardless of rescue treatment
Change from Baseline in Neuro-QoL Pediatric Fatigue regardless of rescue treatment
Change from Baseline in MGFA Post-Interventional Status

These endpoints will be analyzed similarly as described for the secondary endpoints, but based on the FAS population for the entire duration of the study.

### 5.10. Safety Analyses

The safety endpoints of the study are:
Frequency of AEs and SAEs
Frequency of adverse events leading to discontinuation
Incidence of antidrug antibodies (ADA)
Physical examination assessments
Changes from Baseline in vital signs
Change from Baseline in electrocardiogram parameters
Change from Baseline in laboratory assessments

Safety analyses will be performed on the Safety Set.

### 5.10.1. Physical Examinations

The number and percentage of patients with abnormal physical examinations will be summarized by visit.

### 5.10.1.1 Physical Examinations

The number and percentage of patients with abnormal physical examinations will be summarized by visit.

### 5.10.1.2. Vital Signs

Absolute values and change from Baseline in vital signs (including weight and height) will be summarized by visit.

### 5.10.1.3. Adverse Events

Treatment-emergent AEs (serious and nonserious) will be defined as all AEs starting on or after the day of first dose of study drug. Pre-treatment SAEs are any SAEs staring prior to the day of first dose of study drug.

All AEs will be coded using the MedDRA version that is current at the time of the analysis.

Adverse events will be summarized for the first 26 weeks and separately for the entire study by System Organ Class (SOC) and Preferred Term (PT) and, in some cases, by PT only.

### 5.10.2. Clinical Laboratory Tests

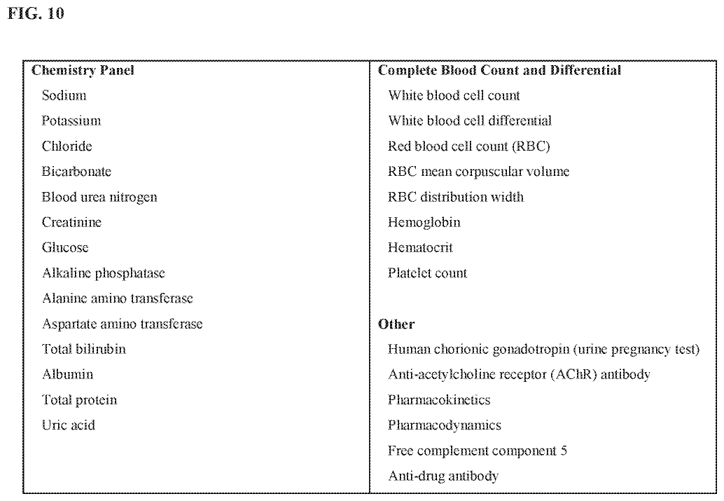

A summary of laboratory panels and tests performed in the clinical trial disclosed herein is shown in FIG. 10. Absolute values and change from Baseline over time in clinical chemistry and hematology results will be summarized descriptively. Laboratory data abnormalities (low, normal, high) with respect to the reference range will be summarized using shift analysis compared to the abnormality at Baseline. Listings of patients with abnormal laboratory values will be provided.

### 5.10.3. Immunogenicity

The number and percentage of patients with positive ADA will be summarized by visit, any time during the first 26 weeks and any time during the study. The proportion of patients ever positive and the proportion of patients always negative may be summarized.

### 5.11. Pharmacokinetic/Pharmacodynamic Analyses

Pharmacokinetic and PD laboratory measurements will be summarized for both Induction and Maintenance Treatment Period. Pharmacokinetic and PD parameter estimates will be modeled and validated. A separate analysis plan will be written for the PK/PD analyses.

The PK/PD endpoints of the study include:
Pharmacokinetic/PD parameters including maximum plasma drug concentration (Cₘₐₓ), terminal half-life (t_{1/2}), trough (C_{trough}), clearance, free C5, and in vitro hemolytic assay; assessed at Baseline and various time points including 24 hours (Day 2), week 12, and week 26 during treatment.

### 5.12. Interim Analyses

The primary analysis of the study for regulatory submission will be performed after all patients complete the 26-week Primary Evaluation Treatment Period. Additional interim analyses may be performed during the Extension Period. The final analysis will be performed when all patients complete the study.

TABLE 22
SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGEILPGSGSTEYTENFKDRVTMT
RDTSTSTVYMELSSLRSEDTAVYYCARYFFGSSPNWYFDVWGQGTLVTVSS
SEQ ID NO: 8
DIQMTQSPSSLSASVGDRVTITCGASENIYGALNWYQQKPGKAPKLLIYGATNLADGVPSRFSGSGSGTDF
TLTISSLQPEDFATYYCQNVLNTPLTFGQGTKVEIK
SEQ ID NO: 9
ASTKGPSVFPLAPCSRSTSESTAALGCLVKDYFPEPVTVSWNSGALTSGVHTFPAVLQSSGLYSLSSVVTV
PSSNFGTQTYTCNVDHKPSNTKVDKTVERKCCVECPPCPAPPVAGPSVFLFPPKPKDTLMISRTTPEVTCV
VVDVSQEDPEVQFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWEWNGQPENNYKTTPPVLDSDGSF
FLYSRLTVDKSRWQEGNVFSCSVMHEALHNHYTQKSLSLSLGK
SEQ ID NO: 10
QVQLVQSGAEVKKPGASVKVSCKASGYIFSNYWIQWVRQAPGQGLEWMGEILPGSGSTEYTENFKDRVTMT
RDTSTSTVYMELSSLRSEDTAVYYCARYFFGSSPNWYFDVWGQGTLVTVSSASTKGPSVFPLAPCSRSTSE
STAALGCLVKDYFPEPVTVSWNSGALTSGVHTFPAVLQSSGLYSLSSVVTVPSSNFGTQTYTCNVDHKPSN
TKVDKTVERKCCVECPPCPAPPVAGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNVFSC
SVMHELAHNHYTQKSLSLSLGK
SEQ ID NO: 11
DIQMTQSPSSLSASVGDRVTITCGASENIYGALNWYQQKPGKAPKLLIYGATNLADGVPSRFSGSGSGTDF
TLTISSLQPEDFATYYCQNVLNTPLTFGQGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
C
SEQ ID NO: 12
QVQLVQSGAEVKKPGASVKVSCKASGHIFSNYWIQWVRQAPGQGLEWMGEILPGSGHTEYTENFKDRVTMT
RDTSTSTVYMELSSLRSEDTAVYYCARYFFGSSPNWYFDVWGQGTLVTVSS
SEQ ID NO: 13
ASTKGPSVFPLAPCSRSTSESTAALGCLVKDYFPEPVTVSWNSGALTSVGHTFPAVLQSSGLYSLSSVVTV
PSSNFGTQTYTCNVDHKPSNTKVDKTVERKCCVECPPCPAPPVAGPSVFLFPPKPKDTLMISRTPEVTCVV
VDVSQEDPEVQFNWYVDGVEVHNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESNGQPENNYKTTPPVLDSDGSFF
LYSRLTVDKSRWQEGNVFSCSVLHEALHSHYTQKSLSLSLGK
SEQ ID NO: 14
QVQLVQSGAEVKKPGASVKVSCKASGHIFSNYWIQWVRQAPGQGLEWMGEILPGSGHTEYTENFKDRVTMT
RDTSTSTVYMELSSLRSEDTAVYYCARYFFGSSPNWYFDVWGQGTLVTVSSASTKGPSVFPLAPCSRSTSE
STAALGCLVKDYFPEPVTVSWNSGALTSGVHTFPAVLQSSGLYSLSSVVTVPSSNFGTQTYTCNVDHKPSN
TKVDKTVERKCCVECPPCPAPPVAGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNVFSC
SVLHEALHSHYTQKSLSLSLGK
SEQ ID NO: 15
ASTKGPSVFPLAPCSRSTSESTAALGCLVKDYFPEPVTVSWNSGALTSGVHTFPAVLQSSGLYSLSSVVTV
TSSNFGTQTYTCNVDHKPSNTKVDKTVERKCCVECPPCPAPPVAGPSVFLFPPKPKDTLYITREPEVTTCV
VVDVSHEDPEVQFNWYVDGMEVHNAKTKPREEQFNSTFRVVSVLTVVHQDWLNGKEYKCKVSNKGLPAPIE
KTISKTKGQPREPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
SEQ ID NO: 16
QVQLVQSGAEVKKPGASVKVSCKASGYIFSNYWIQWVRQAPGQGLEWMGEILPGSGSTEYTENFKDRVTMT
RDTSTSTVYMELSSLRSEDTAVYYCARYFFGSSPNWYFDVWGQGTLVTVSSASTKGPSVFPLAPCSRSTSE
STAALGCLVKDYFPEPVTVSWNSGALTSGVHTFPAVLQSSGLYSLSSVVTVTSSNFGTQTYTCNVDHKPSN
TKVDKTVERKCCVECPPCPAPPVAGPSVFLFPPKPKDTLYITREPEVTCVVVDVSHEDPEVQFNWYVDGME
VHNAKTKPREEQFNSTFRVVSVLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSFFLYSKLTVDKSRWQQGNVFSC
SVMHEALHNHYTQKSLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGEILPGSGHTEYTENFKDRVTMT
RDTSTSTVYMELSSLRSEDTAVYYCARYFFGSSPNWYFDVWGQGTLVTVSSASTKGPSVFPLAPCSRSTSE
STAALGCLVKDYFPEPVTVSWNSGALTSGVHTFPAVLQSSGLYSLSSVVTVPSSNFGTQTYTCNVDHKPSN
TKVDKTVERKCCVECPPCPAPPVAGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSFFLYSRLTVDKSRWQEGNVFSC
SVMHEALHNHYTQKSLSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGAEVKKPGSSVKVSCKASGGTFSSYAISVWRQAPGQGLEWMGGIGPFFGTANYAQKFQGRVTIT
ADESTSTAYMELSSLRSEDTAVYYCARDTPYFDYWGQGTLVTVSS
SEQ ID NO: 28
DIELTQPPSVSVAPGQTARISCSGDSIPNYYVYWYQQKPGQAPVLVIYDDSNRPSGIPERFSGSNSGNTAT
LTISGTQAEDEADYYCQSFDSSLNAEVFGGGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGIIDPDDSYTEYSPSFQGQVTISA
DKSISTAYLQWSSLKASDTAMYYCARYEYGGFDIWGQGTLVTVSS
SEQ ID NO: 36
SYELTQPPSVSVAPGQTARISCSGDNIGNSYVHWYQQKPGQAPVLVIYKDNDRPSGIPERFSGSNSGNTAT
LTISGTQAEDEADYYCGTYDIESYVFGGGTKLTVL
SEQ ID NO: 37
QVQLVESGGGLVQPGRSLRLSCAASGFTVHSSYYMAWVRQAPGKGLEWVGAIFTGSGAEYKAEWAKGRVTI
SKDTSKNWVVLTMTNMDPVDTATYYCASDAGYDYPTHAMHYWGQGTLVTVSS
SEQ ID NO: 38
DIQMTQSPSSLSASVGDRVTITVRASQGISSSLAWYQQKPGKAPKLLIYGASETESGVPSRFGSGSGTDFT
LTISSLQPEDFATYYCQNTKVGSSYGNTFGGGTKVEIK
SEQ ID NO: 39
QVQLVESGGGLVQPGRSLRLSCAASGFTVHSSYYMAWVRQAPGKGLEWVGAIFTGSGAEYKAEWAKGRVTI
SKDTSKNQVVLTMTNMDPVDTATYYCASDAGYDYPTHAMHYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELRRGPKVFLFPPKPKDTLMISRTPEVTCVVVDVSHEDPEVKFNWY
VDGVEVHNAKTKPREEQYNSTYRVVSVLTVLHQDWLNGKEYKCKVSNKGLPSSIEKTISKAKGQPREPQVY
TLPPSREEMTKNQVSLTCLVKGFYPSDIAVEWESNGQPENNYKTTPPVLDSDGSFFLYSKLTVDKSRWQQG
NVFSCSVLHEALHAHYTRKELSLSP

### REFERENCES CITED

The following references are hereby incorporated by reference in their entirety:
(1) Conti-Fine et al., “Myasthenia gravis: past, present, and future,” J. Clin. Invest. 116(11): 2843-54 (2006).
(2) Howard J. F., “Clinical Overview of MG,” Myasthenia Gravis Foundation of America, Inc. 2006.
(3) Phillips, “The epidemiology of myasthenia gravis,” Semin. Neurol. 24(1): 17-20(2004).
(4) Kim et al., “Treatment of myasthenia gravis based on its immunopathogenesis,” J. Clin. Neurol. 7(4): 173-83 (2011).
(5) Vincent et al., “Myasthenia gravis,” Adv. Neurol. 88: 159-88 (2002).
(6) Sahashi et al., “Ultrastructural localization of immune complexes (IgG and C3) at the end-plate in experimental autoimmune myasthenia gravis. J. Neuropathol. Exp. Neurol. 37(2): 212-23 (1978).
(7) Dalakas, “Intravenous immunoglobulin in autoimmune neuromuscular diseases,” JAMA 291(19): 2367-75 (2004).
(8) Engel et al., “Immune complexes (IgG and C3) at the motor end-plate in myasthenia gravis: ultrastructural and light microscopic localization and electrophysiologic correlations,” Mayo Clin. Proc. 52(5): 267-80 (1977).
(9) Nastuk et al., “Changes in serum complement activity in patients with myasthenia gravis,” Proc. Soc. Exp. Biol. Med. 105: 177-84 (1960).
(10) Peng et al., “Role of C5 in the development of airway inflammation, airway hyperresponsiveness, and ongoing airway response,” J. Clin. Invest. 115(6): 1590-600 (2005).
(11) Vakeva et al., “Myocardial infarction and apoptosis after myocardial ischemia and reperfusion: role of the terminal complement components and inhibition by anti-C5 therapy,” Circulation 97(22): 2259-67 (1998).
(12) Wang et al., “Complement inhibition with an anti-C5 monoclonal antibody prevents hyperacute rejection in a xenograft heart transplantation model,” Transplantation 68(11): 1643-51 (1999).
(13) Howard et al., “A randomized, double-blind, placebo-controlled phase II study of eculizumab in patients with refractory generalized myasthenia gravis,” Muscle Nerve 48(1): 76-84 (2013).
(14) Muppidi et al., “MG-ADL: still a relevant outcome measure,” Muscle Nerve 44(5): 727-31 (2011).
(15) Benatar et al., “Recommendations for myasthenia gravis clinical trials,” Muscle Nerve 45(6): 909-17 (2012).
(16) Burns et al., “The MG Composite: A valid and reliable outcome measure for myasthenia gravis,” Neurology 74(18): 1434-40 (2010).
(17) Burns et al., “The MG-QOL15 for following the health-related quality of life of patients with myasthenia gravis,” Muscle Nerve 43(1): 14-8 (2011).
(18) Cella D., Measuring Quality of Life in Neurological Disorders; Final Report of the Neuro-QOL Study September 2010. 2010.
(19) Szende A. and Williams A., “Measuring Self-Reported Population Health: An International Perspective based on EQ-5D, (2004) <http://www.euroqol.org/fileadmin/user_upload/Documenten/PDF/Books/Measuring_Self-Reported_Population_Health_-_An_International_Perspective_based_on_EQ-5D.pdf>
(20) Posner et al., “The Columbia-Suicide Severity Rating Scale: initial validity and internal consistency findings from three multisite studies with adolescents and adults,” Am. J. Psychiatry 168(12): 1266-77 (2011).
(21) Nilsson el al., “Columbia-Suicide Severity Rating Scale Scoring and Data Analysis Guide, (2013)<http://cssrs.columbia.edu/wp-content/uploads/ScoringandDataAnalysisGuide-for-Clinical-Trials.pdf>.
(22) Jin F, Balthasar J P. Mechanisms of intravenous immunoglobulin action in immune thrombocytopenic purpura. Hum Immunol. 2005; 66(4):403-410.
(23) Wang W, Wang E Q, Balthasar J P. Monoclonal antibody pharmacokinetics and pharmacodynamics. Clin Pharmacol Ther. 2008; 84(5):548-558.
(24) Fitzpatrick A M, Mann C A, Barry S, Brennan K, Overell J R, Willison H J. An open label clinical trial of complement inhibition in multifocal motor neuropathy. J Peripher Nerv Syst. 2011; 16(2):84-91.
(25) Goldstein S D, Culbertson N T, Garrett D, et al. Thymectomy for myasthenia gravis in children: a comparison of open and thoracoscopic approaches. J Pediatr Surg. 2015:50(1):92-97.
(26) Szende A, Janssen B, Cabasés, J. Self-reported population health: an international perspective based on EQ-5D. 2014.
(27) Andrews P I. Autoimmune myasthenia gravis in childhood. Semin Neurol. 2004; 24(1):101-110.
(28) Barnett C, Bril V, Kapral M, Kulkarni A, David A M. A conceptual framework for evaluating impairments in myasthenia gravis. PLoS One. 2014; 9(5):e98089.
(29) Della Marina A, Trippe H, Lutz S, Schara U. Juvenile myasthenia gravis: recommendations for diagnostic approaches and treatment. Neuropediatrics. 2014:45(2):75-83.
(30) McGrogan A, Sneddon S, de Vries C S. The incidence of myasthenia gravis: a systematic literature review. Neuroepidemiology. 2010; 34(3):171-183.
(31) Phillips L H 2nd, Torner J C, Anderson M S, Cox G M. The epidemiology of myasthenia gravis in central and western Virginia. Neurology. 1992; 42(10):1888-1893.
(32) Sieb, J P. Myasthenia gravis: an update for the clinical. Clin Exp Immunol. 2014; 175(3):408-418.
(33) Snead O C 3rd, Benton J W, Dwyer D, et al. Juvenile myasthenia gravis. Neurology. 1980; 30(7 Pt 1):732-739.
(34) Szorbor A, Máttyus A, Molnár J. Myasthenia gravis in childhood and adolescence: report on 209 patients and review of the literature. Acta Paediatr Hung. 1988-1989; 29(3-4):299-312.

What is claimed is:

1. Eculizumab for use in the treatment of refractory generalized myasthenia gravis in a pediatric patient in need thereof comprising administering a therapeutically effective amount of eculizumab to the patient for at least 26 weeks.

2. The use of claim 1, wherein eculizumab is administered using a phased dosing schedule comprising an induction phase and a maintenance phase, wherein the induction phase comprises administering between 300 mg to 1200 mg of eculizumab to the patient every week.

3. The use of claim 1, wherein eculizumab is administered using a phased dosing schedule comprising an induction phase and a maintenance phase, wherein for a patient weighing ≥10 and <20 kg, the induction phase comprises administering a first dose of 600 mg eculizumab to the patient on Day 1.

4. The use of claim 1, wherein eculizumab is administered using a phased dosing schedule comprising an induction phase and a maintenance phase, wherein for a patient weighing ≥20 and <30 kg, the induction phase comprises administering a first dose of 600 mg eculizumab to the patient on Day 1, and a second dose of 600 mg eculizumab at least 7 days thereafter.

5. The use of claim 1, wherein eculizumab is administered using a phased dosing schedule comprising an induction phase and a maintenance phase, wherein for a patient weighing ≥30 and <40 kg, the induction phase comprises administering a first dose of 600 mg eculizumab to the patient on Day 1, and a second dose of 600 mg eculizumab at least 7 days thereafter.

6. The use of claim 1, wherein eculizumab is administered using a phased dosing schedule comprising an induction phase and a maintenance phase, wherein for a patient weighing ≥40 kg, the induction phase comprises administering a first dose of eculizumab 900 mg to the patient on Day 1, and a second dose, a third dose, and a fourth dose of 900 mg eculizumab 7, 14, and 21 days thereafter, respectively.

7. The use of claim 2, wherein the induction phase of eculizumab treatment is followed by a maintenance phase comprising administering 300 mg to 1200 mg of eculizumab to the patient every two weeks.

8. The use of any one of claims 2-6, wherein the induction phase of eculizumab treatment is followed by a maintenance phase, wherein for a patient weighing ≥10 and <20 kg, the maintenance phase comprises administering a first dose of 300 mg eculizumab to the patient at Week 1, and subsequent doses of 300 mg eculizumab every 2 weeks thereafter.

9. The use of any one of claims 2-6, wherein the induction phase of eculizumab treatment is followed by a maintenance phase, wherein for a patient weighing ≥20 and <30 kg, the maintenance phase comprises administering a first dose of 600 mg eculizumab to the patient at Week 2, and subsequent doses of 600 mg eculizumab every 2 weeks thereafter.

10. The use of any one of claims 2-6, wherein the induction phase of eculizumab treatment is followed by a maintenance phase, wherein for a patient weighing ≥30 and <40 kg, the maintenance phase comprises administering a first dose of 900 mg eculizumab to the patient at Week 2, and subsequent doses of 900 mg eculizumab every 2 weeks thereafter.

11. The use of any one of claims 2-6, wherein the induction phase of eculizumab treatment is followed by a maintenance phase, wherein for a patient weighing ≥40 kg, the maintenance phase comprises administering a first dose of 1200 mg eculizumab to the patient at Week 4, and subsequent doses of 1200 mg eculizumab every 2 weeks thereafter.

12. The use of claim 1, wherein the patient experiences a clinically meaningful improvement (reduction) in Myasthenia Gravis Activities of Daily Living (MG-ADL) score after 26 weeks of treatment.

13. The use of claim 1, wherein the patient experiences a clinically meaningful improvement (reduction) in quantitative Myasthenia Gravis score (QMG) after 26 weeks of treatment.

14. The use of claim 1, wherein the patient experiences a clinically meaningful improvement (reduction) in Myasthenia Gravis Composite (MGC) score after 26 weeks of treatment.

15. The use of claim 1, wherein the patient experiences a clinically meaningful improvement (reduction) in quality of life as measured by Myasthenia Gravis Quality of Life (MG-QOL-15) score after 26 weeks of treatment.

16. The use of claim 1, wherein the patient experiences a clinically meaningful improvement (reduction) in neuro-fatigue as measured by Neuro-QOL Fatigue score after 26 weeks of treatment.

17. The use of claim 1, wherein the patient experiences a clinically meaningful improvement (increase) in health status as measured by EQ-5D health status score after 26 weeks of treatment.

18. The use of claim 1, wherein eculizumab is administered by intravenous infusion.

19. The use of claim 1, wherein eculizumab is administered subcutaneously.

20. The use of claim 1, wherein the eculizumab comprises a heavy chain amino acid sequence according to SEQ ID NO: 10 and a light chain amino acid sequence according to SEQ ID NO: 11.

21. The use of claim 1, wherein the eculizumab is an eculizumab variant comprising a heavy chain amino acid sequence according to SEQ ID NO: 14 and a light chain amino acid sequence according to SEQ ID NO: 11.

22. The use of claim 1, wherein the patient has failed treatment over one year or more with two or more ISTs in sequence or in combination.

23. The method of claim 1, wherein the patient has failed at least one IST and requires chronic plasma exchange or IVIg to control symptoms.

24. The use of claim 1, wherein the therapeutically effective amount of eculizumab is maintained at a concentration of between 50-100 μg/mL in the patient's serum.

25. The use of claim 1, wherein the patient experiences a reduction in the administration of one or more IST following at least 26 weeks of treatment.

26. The use of claim 1, wherein the patient experiences a reduction in IST dosing following at least 26 weeks of treatment.

27. The use of claim 1, wherein the patient experiences a reduction in one or more IST dosing and a discontinuation in one or more IST following at least 26 of treatment.

28. The use of claim 1, wherein the patient has a QMG total score ≥12 prior to administering the therapeutically effective amount of eculizumab to the patient.


SEQUENCE LISTING (39 sequences)

SEQ ID NO: 1 - protein, 10 aa, Artificial Sequence
  Heavy chain CDR sequence 1
GYIFSNYWIQ

SEQ ID NO: 2 - protein, 17 aa, Artificial Sequence
  Heavy chain CDR sequence 2
EILPGSGSTEYTENFKD

SEQ ID NO: 3 - protein, 13 aa, Artificial Sequence
  Heavy chain CDR sequence 3
YFFGSSPNWYFDV

SEQ ID NO: 4 - protein, 11 aa, Artificial Sequence
  Light chain CDR sequence 1
GASENIYGALN

SEQ ID NO: 5 - protein, 7 aa, Artificial Sequence
  Light chain CDR sequence 2
GATNLAD

SEQ ID NO: 6 - protein, 9 aa, Artificial Sequence
  Light chain CDR sequence 3
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Heavy chain constant region sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Heavy chain sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Heavy chain constant region sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Heavy chain sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Heavy chain constant region sequence
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Heavy chain sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  Light chain CDR sequence 1
GASENIYHALN

SEQ ID NO: 18 - protein, 17 aa, Artificial Sequence
  Heavy chain CDR sequence 2
EILPGSGHTEYTENFKD

SEQ ID NO: 19 - protein, 10 aa, Artificial Sequence
  Heavy chain CDR sequence 1
GHIFSNYWIQ

SEQ ID NO: 20 - protein, 448 aa, Artificial Sequence
  Heavy chain sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  Heavy chain CDR sequence 1
SYAIS

SEQ ID NO: 22 - protein, 17 aa, Artificial Sequence
  Heavy chain CDR sequence 2
GIGPFFGTANYAQKFQG

SEQ ID NO: 23 - protein, 7 aa, Artificial Sequence
  Heavy chain CDR sequence 3
DTPYFDY

SEQ ID NO: 24 - protein, 11 aa, Artificial Sequence
  Light chain CDR sequence 1
SGDSIPNYYVY

SEQ ID NO: 25 - protein, 7 aa, Artificial Sequence
  Light chain CDR sequence 2
DDSNRPS

SEQ ID NO: 26 - protein, 11 aa, Artificial Sequence
  Light chain CDR sequence 3
QSFDSSLNAEV

SEQ ID NO: 27 - protein, 116 aa, Artificial Sequence
  Heavy chain variable region sequence
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Light chain variable region sequence
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  Heavy chain CDR sequence 1
NYIS

SEQ ID NO: 30 - protein, 17 aa, Artificial Sequence
  Heavy chain CDR sequence 2
IIDPDDSYTEYSPSFQG

SEQ ID NO: 31 - protein, 8 aa, Artificial Sequence
  Heavy chain CDR sequence 3
YEYGGFDI

SEQ ID NO: 32 - protein, 11 aa, Artificial Sequence
  Light chain CDR sequence 1
SGDNIGNSYVH

SEQ ID NO: 33 - protein, 7 aa, Artificial Sequence
  Light chain CDR sequence 2
KDNDRPS

SEQ ID NO: 34 - protein, 9 aa, Artificial Sequence
  Light chain CDR sequence 3
GTYDIESYV

SEQ ID NO: 35 - protein, 116 aa, Artificial Sequence
  Heavy chain variable region sequence
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  Light chain variable region sequence
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 123 aa, Artificial Sequence [sequence omitted]

SEQ ID NO: 38 - protein, 110 aa, Artificial Sequence [sequence omitted]

SEQ ID NO: 39 - protein, 451 aa, Artificial Sequence
  Heavy chain sequence
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP